(19) United States
(12) Patent Application Publication
(10) Pub. No.: US 2022/0157403 A1
(43) Pub. Date: May 19, 2022

(54) SYSTEMS AND METHODS TO CLASSIFY ANTIBODIES

(71) Applicant: ETH ZURICH, Zurich (CH)

(72) Inventors: Derek Mason, Basel (CH); Simon FRIEDENSOHN, Riehen (CH); Cédric Weber, Zurich (CH); Sai REDDY, Basel (CH)

(21) Appl. No.: 17/439,374

(22) PCT Filed: Apr. 8, 2020
(86) PCT No.: PCT/IB2020/053370

§ 371 Date: Sep. 14, 2021

Related U.S. Application Data
(60) Provisional application No. 62/831,663, filed on Apr. 9, 2019.

(51) Int. Cl. G16B 40/20; G16B 20/20; G16B 20/30; G06N 5/02

(52) U.S. Cl. CPC G16B 40/20; G06N 5/022; G16B 20/30; G16B 20/20

(57) ABSTRACT

The present disclosure describes systems and methods to make predictions classifying one or more properties of a binding protein such as an antibody, for example, antibody affinity or specificity for an antigen. The system can include one or more machine learning models that can extrapolate complex relationships between amino acid sequence and function. The system can be trained on high-quality training data generated through a two-step single-site and combinatorial deep mutational scanning approach. The trained models can then make predictions on novel variant sequences generated in silico. The present disclosure describes amino acid sequences generated by the systems and methods provided, and uses of the generated sequences to produce proteins for therapeutic and diagnostic use.

### CROSS-REFERENCE TO RELATED APPLICATIONS

This application is a U.S. National Stage under 35 U.S.C. § 371 of International Patent Application No. PCT/IB2020/053370, filed Apr. 8, 2020 and designating the United States, which claims priority under 35 U.S.C. § 119 to U.S. Provisional Patent Application No. 62/831,663 filed Apr. 9, 2019, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jul. 15, 2020, is named 122043-0104 SL.txt and is 42,289 bytes in size.

### BACKGROUND OF THE DISCLOSURE

In antibody drug discovery, screening of phage or yeast display libraries is a standard practice for identifying therapeutic antibodies and can typically result in a number of potential lead variant candidates. However, the time and costs associated with lead candidate optimization often take up the majority of the drug preclinical discovery and development cycle. This is largely due to the fact that lead optimization of antibody molecules frequently includes addressing multiple parameters in parallel, including expression level, viscosity, pharmacokinetics, solubility, and immunogenicity. Once a lead candidate is discovered, additional engineering is often required. The fact that nearly all therapeutic antibodies require expression in mammalian cells as full-length IgG also means that the remaining development and optimization steps must occur in this context. Since mammalian cells lack the capability to replicate plasmids stably, this last stage of development is done at low-throughput, as elaborate cloning, transfection and purification strategies must be implemented to screen libraries in the max range of about 10³ antibody molecules. This can result in only minor changes (e.g., single point mutations) being screened. Interrogating such a small fraction of protein sequence space also implies that addressing one development issue can frequently cause the rise of another or even diminish antigen binding altogether, making multi-parameter optimization challenging.

### SUMMARY OF THE DISCLOSURE

Provided herein are systems and methods for the classification of amino acid sequences of binding proteins, including, for example, an antibody that binds to an antigen or a receptor that binds to a ligand. In some embodiments, the methods provided herein combine directed evolution with machine learning to develop new proteins based on an input amino acid sequence. In some embodiments, the methods provided can identify an amino acid sequence that improves one or more properties the binding protein, for example, an increase in the affinity or specificity of an antibody binding to an antigen, or two or more antigens (e.g., multispecific).

According to at least one aspect of the disclosure, a method can include providing an input amino acid sequence that represents a portion of a binding protein. In some embodiments, the portion is an antigen binding portion of an antibody. In some embodiments, the portion affects one or more properties of the binding protein (e.g., antigen binding affinity). The method can include generating a first training data set comprising a first plurality of variant sequences. Each of the first plurality of sequences can include a single site mutation in the input amino acid sequence of the binding protein (e.g., an antibody). The method can include generating a second training data set comprising a second plurality of sequences. Each of the second plurality of sequences can include a plurality of variants at positions based on enrichment scores of the first training data set comprising the first plurality of sequences. The method can include providing the second training data set to a classification engine comprising a first machine learning model to generate a plurality of parameters for the first machine learning model. The method can include determining, by the classification engine based on the plurality of parameters for the first machine learning model, a first affinity binding score for a proposed amino acid sequence to an antigen. In some embodiments, the parameters comprise weights and biases for the first learning model. The method can include selecting the proposed amino acid sequence for further analysis and validation and/or expression based on the first affinity binding score satisfying a threshold. In some embodiments, further analysis and validation of the proposed amino acid sequence is based on one more parameters related to the developability and/or therapeutic potential of the proposed amino acid sequence.

The method can include determining, by the classification engine, a second affinity binding score for the proposed amino acid sequence using a second machine learning model of the classification engine. The method can include selecting the proposed amino acid sequence for expression based on the first affinity binding score and the second affinity binding score satisfying the threshold. The method can include determining, by the classification engine, an affinity binding score for each of a plurality of proposed amino acid sequences. The method can include determining, by a candidate selection engine, one or more parameters for each of the plurality of proposed amino acid sequences. The method can include selecting, by the candidate selection engine, candidate variants from the plurality of proposed amino acid sequences based on the affinity binding score and the one or more parameters for each of the plurality of proposed amino acid sequences. The one or more parameters can include protein sequence based metrics such as the Levenshtein distance value, charge value, hydrophobicity index value, CamSol score, minimum affinity rank, or average affinity ranking. The protein sequence based metrics can also include sequence motifs associated with manufacturing liabilities, such as n-glycosylation sites, deamidation sites, isomerization sites, methionine oxidation, tryptophan oxidation and paired or unpaired cysteine residues. The one or more parameters can also include protein structured based metrics such as the solvent accessible surface area (SASA), patches positive charges (PPC), patches negative charges (PNC), patches surface hydrophobicity (PSH) and surface Fv charge symmetry parameter (SFvCSP).

The first machine learning model can include a recurrent neural network (RNN), a convolutional neural network (CNN), a standard artificial neural network (ANN), a support vector machine (SVM), a random forest ensemble (RF) or logistic regression (LR) model.

The input amino acid sequence can be a portion of a complementarity determining region (CDR) of the antibody. The input amino acid sequence can be a CDRH1, CDRH2, CDRH3, CDRL1, CDRL2, CDRL3, a region within the framework domains of the antibody (e.g., FR1, FR2, FR3, FR4) or a region within the constant domains of the antibody (e.g., CH1, CH2, CH3), or any combination thereof, for which improvement of one or more properties of the antibody is desired. The input amino acid sequence can be a full length heavy chain or a full length light chain. The input amino acid sequence can be a recombinant sequence comprising one or more portion of an antibody. The antibody can be a therapeutic antibody. The first training data set can be generated by deep mutational scanning. The deep mutational scanning can include generating a first library of variant sequences wherein each variant sequence is modified at a single amino acid position relative to the input amino acid sequence. The first library can include variant sequences representing each amino acid position of the input amino acid sequence.

The first library can include variant sequences representing all 20 amino acids at each position of the input amino acid sequence. The first library of variant sequences can be generated by mutagenesis of the nucleic acid sequences encoding the input amino acid sequence. The first library of variant sequences can be generated by mutagenesis and introduction of the mutant sequences into a suitable expression system. The mutagenesis method can include any suitable method, such as error-prone PCR, recombination mutagenesis, alanine scanning mutagenesis, structure-guided mutagenesis, or homology-directed repair (HDR). The expression system can be, for example, a mammalian, yeast, bacteria, or phage expression system. The first library of variant sequences can be generated by high throughput mutagenesis in a mammalian cell. The first library of variant sequences can be generated by CRISPR/Cas9-mediated homology-directed repair (HDR). The deep mutational scanning can include generating a plurality of antibodies that can include the first library of variant sequences. The deep mutational scanning can include screening the plurality of antibodies and the first library of variant sequences for binding to an antigen and determining the sequence and frequency of variants selected for binding to the antigen, thereby obtaining the first training data set.

The second training data set can be generated by deep mutational scanning-guided combinatorial mutagenesis. The deep mutational scanning-guided combinatorial mutagenesis can include generating a second library of variant sequences wherein each variant sequence is modified at two or more amino acid positions based on the first training data set. The second library of variant sequences can be generated by high throughput mutagenesis in a mammalian cell. The second library of variant sequences is generated by CRISPR/Cas9-mediated homology-directed repair (HDR). The deep mutational scanning-guided combinatorial mutagenesis can include generating a plurality of antibodies comprising the second library of variant sequences. The combinatorial deep mutational scanning can include screening the plurality of antibodies that can include the second library of variant sequences for binding to the antigen and determining the sequence of variants selected for binding to the antigen, thereby obtaining the second training data set.

Also provided herein are proteins or peptides comprising an amino acid sequence generated by the methods provided herein. In some embodiments, the generated amino acid sequence is a CDRH3. In some embodiments, the protein or peptide comprising an amino acid sequence generated herein is an antibody or fragment thereof. In some embodiments, the protein or peptide comprising an amino acid sequence generated herein is a full length antibody. In some embodiments, the protein or peptide comprising an amino acid sequence generated herein is a fusion protein comprising one or more portions of an antibody. In some embodiments, the protein or peptide comprising an amino acid sequence generated herein is an scFv or an Fc fusion protein. In some embodiments, the protein or peptide comprising an amino acid sequence generated herein is a chimeric antigen receptor. In some embodiments, the protein or peptide comprising an amino acid sequence generated herein is a recombinant protein. In some embodiments, the protein or peptide comprising an amino acid sequence generated herein binds to an antigen. In some embodiments, the antigen is associated with a disease or condition. In some embodiments, the antigen is a tumor antigen, an inflammatory antigen, pathogenic antigen (e.g., viral, bacterial, yeast, parasitic). In some embodiments, the protein or peptide comprising an amino acid sequence generated herein has one or more improved properties compared to a protein or peptide comprising the input amino acid sequence. In some embodiments, the protein or peptide comprising an amino acid sequence generated herein has improved affinity for an antigen compared to a protein or peptide comprising the input amino acid sequence. In some embodiments, the protein or peptide comprising an amino acid sequence generated herein has improved biophysical properties for manufacturing compared to a protein or peptide comprising the input amino acid sequence. In some embodiments, the protein or peptide comprising an amino acid sequence generated herein has reduced immunogenic risk compared to a protein or peptide comprising the input amino acid sequence. In some embodiments, the protein or peptide comprising an amino acid sequence generated herein can be administered to treat an inflammatory disease, infectious disease, cancer, genetic disorder, organ transplant rejection, autoimmune disease or an immunological disorder. In some embodiments, the protein or peptide comprising an amino acid sequence generated herein can be used for the manufacture of a medicament to treat an inflammatory disease, infectious disease, cancer, genetic disorder, organ transplant rejection, autoimmune disease and immunological disorder. Also provided herein are cells comprising one more proteins or peptides comprising an amino acid sequence generated herein. The cell can be a mammalian cell, a bacterial cell, a yeast cell or any cell that can express a protein or peptide comprising an amino acid sequence generated herein. The cell can be an immune cell, such as a T cell (e.g., a cell used in Chimeric Antigen Receptor (CAR) T-cell therapy). In some embodiments, the protein or peptide comprising an amino acid sequence generated herein can be used to detect an antigen in a biological sample.

Also provided herein are proteins or peptides comprising an amino acid sequence shown any of FIGS. 15A-D, 23 A-O. In some embodiments, the amino acid sequence shown any of FIGS. 15A-D, 23 A-O is a CDRH3. In some embodiments, the protein or peptide comprising an amino acid sequence shown any of FIGS. 15A-D, 23 A-O is an antibody or fragment thereof. In some embodiments, the protein or peptide comprising an amino acid sequence shown any of FIGS. 15A-D, 23 A-O is a full length antibody. In some embodiments, the protein or peptide comprising an amino acid sequence shown any of FIGS. 15A-D, 23 A-O is a fusion protein comprising one or more portions of an antibody. In some embodiments, the protein or peptide comprising an amino acid sequence shown any of FIG. 15A-D, 23 A-O is an scFv or an Fc fusion protein. In some embodiments, the protein or peptide comprising an amino acid sequence shown any of FIGS. 15A-D, 23 A-O is a chimeric antigen receptor. In some embodiments, the protein or peptide comprising an amino acid sequence shown any of FIGS. 15A-D, 23 A-O is a recombinant protein. In some embodiments, the protein or peptide comprising an amino acid sequence shown any of FIGS. 15A-D, 23 A-O binds to the HER2 (human epidermal growth factor receptor 2) antigen. In some embodiments, the protein or peptide comprising an amino acid sequence shown any of FIGS. 15A-D, 23 A-O has one or more improved properties compared to the trastuzumab (Herceptin) antibody. In some embodiments, the protein or peptide comprising an amino acid sequence shown any of FIGS. 15A-D, 23 A-O has improved affinity for the HER2 antigen compared to the trastuzumab (Herceptin) antibody. In some embodiments, the protein or peptide comprising an amino acid sequence shown any of FIGS. 15A-D, 23 A-O can be administered to treat a HER2 positive cancer. In some embodiments, the protein or peptide comprising an amino acid sequence shown any of FIGS. 15A-D, 23 A-O can be administered to treat a HER2 positive breast cancer. In some embodiments, the protein or peptide comprising an amino acid sequence shown any of FIGS. 15A-D, 23 A-O can be used for the manufacture of a medicament to treat a HER2 positive breast cancer. In some embodiments, the HER2 positive cancer is a metastatic cancer. Also provided herein are cells comprising one more proteins or peptides comprising an amino acid sequence shown any of FIGS. 15A-D, 23 A-O. The cell can be a mammalian cell, a bacterial cell, a yeast cell or any cell that can express a protein or peptide comprising an amino acid sequence shown any of FIGS. 15A-D, 23 A-O. The cell can be an immune cell, such as a T cell (e.g., a CAR-T cell). In some embodiments, the protein or peptide comprising an amino acid sequence shown any of FIGS. 15A-D, 23 A-O can be used to detect a HER2 antigen in a biological sample.

The foregoing general description and the following description of the drawings and detailed description are exemplary and explanatory and are intended to provide further explanation of the invention as claimed. Other objects, advantages, and novel features will be readily apparent to those skilled in the art from the following brief description of the drawings and detailed description.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 illustrates a block diagram of an example system to select antibody candidates.

FIG. 2A illustrates an example neural network that can be used with the example system illustrated in FIG. 1. FIG. 2A discloses SEQ ID NO: 1.

FIG. 2B illustrates an example receiver operating characteristic.

FIG. 3A illustrates another example neural network that can be used with the example system illustrated in FIG. 1. FIG. 3A discloses SEQ ID NO: 1.

FIG. 3B illustrates an example receiver operating characteristic.

FIG. 4A illustrates an example flow process for generating training data that can be used with the example system illustrated in FIG. 1. FIG. 4A discloses SEQ ID NO: 2.

FIG. 4B illustrates an example flow process for selecting candidate variants using the example system illustrated in FIG. 1. FIG. 4B discloses SEQ ID NOS 3 and 1, respectively, in order of appearance.

FIG. 5A illustrates (A) the Trastuzumab (Herceptin) CDRH3 variant sequence and (B) Flow cytometry profile following the integration of tiled mutations by homology-directed mutagenesis. FIG. 5A discloses SEQ ID NOS 4-25, respectively, in order of appearance.

FIG. 5B illustrates antigen-specific variants that underwent 3 rounds of enrichment (C) Corresponding heatmap following sequencing analysis of the pre-sorted (Ab+) and post-sorted (Ag+) populations. Black circles mark wild type amino acids. (D) The resulting sequence logo plot generated by positively enriched mutations per position.

FIG. 5C illustrates (E) 3D protein structure of trastuzumab in complex with its target antigen, HER2 (Cho et al. (2003) Nature 421 (6924): 756-60). Locations of surface-exposed amino acid positions: 102D, 103G, 104F, and 105Y are provided.

FIG. 6A illustrates (A) Sequence logo plot and (B) Flow cytometry plots resulting from transfection of a rationally designed library. Two rounds of enrichment were performed to produce a library of antigen-specific variants.

FIG. 6B illustrates how next-generation sequencing was performed on the library (Ab+), non-binding variants (Ag−), and binding variants after 1 and 2 rounds of enrichment (Ag+1, Ag+2) (C, D) Amino acid frequency plots of (C) antigen binding variants and (D) non-binding variants reveals nearly indistinguishable amino acid usages across all positions. FIG. 6B discloses SEQ ID NO: 26.

FIGS. 7A-E illustrate an example filtering policy that can be used with the example system illustrated in FIG. 1. Histograms show the parameters distributions of all predicted variants at the different stages of filtering. FIG. 7A illustrates (A) Levenshtein distance from wild-type trastuzumab; and (B) Net charge of the VH domain. FIG. 7B illustrates (C) CDRH3 hydrophobicity index; and (D) CamSol intrinsic solubility score. FIG. 7C illustrates (E) Minimum NetMHCIIpan % Rank across all possible 15-mers; and (F) Average NetMHCIIpan % Rank across all possible 15-mers. FIG. 7D illustrates (G) count numbers for sequences with various average netMHC scores; and (H) overall developability scores for experimental and predicted binders. FIG. 7E illustrates (I) filtering parameters and the number of sequences at the corresponding stage of filtering

FIG. 8 illustrates a block diagram of an example method to identify antibodies with an antigen affinity using the example system illustrated in FIG. 1.

FIGS. 9A-9B illustrate the Trastuzumab (Herceptin) CDRH3 variant and CDRH3 sequence and flow cytometry data following transfection of the hybridoma cells with either gRNA only (bottom left panel), gRNA+DMS ssODN library (bottom middle panel), or gRNA+DMS-combinatorial mutagenesis library (bottom right panel). The top middle panel is a representative flow cytometry plot of the Trastuzumab CDRH3 variant prior to transfection. FIG. 9A discloses SEQ ID NOS 27-28, respectively, in order of appearance.

FIG. 10 illustrates exemplary flow cytometry data for Trastuzumab (Herceptin) CDRH3 deep mutation scanning. (A) Flow cytometry plot, heatmap, and sequence logo plot following FACS for antibody expressing (Ab+) cells and antigen-specific (Ag+) cells. (B) Flow cytometry plot, heatmap, and sequence logo plot following a second round of enrichment for antigen-specific (Ag+2) cells; Decreased antigen concentration was used for flow cytometry labeling. (C) Flow cytometry plot, heatmap, and sequence logo plot following a third round of enrichment for antigen-specific (Ag+3) cells; Labeling for flow cytometry performed with antigen containing an alternatively conjugated fluorophore (Alexa Fluor 488). All enrichment ratios (ER) are calculated by dividing the frequency of a mutant found in the respective Ag+ population by the frequency of the mutant found in the Ab+ population.

FIG. 11 illustrates exemplary workflow and flow cytometry data for generating antigen specific libraries in mammalian cells. Libraries are generated by transfecting gRNA and ssODN donor templates containing rationally designed libraries. Antibody expressing cells (Ab+) are enriched by magnetic activated cell sorting (MACS). Ab+ cells can then undergo multiple rounds of enrichment for antigen-specific variants. Antigen-specific libraries are designed from enrichment ratios calculated following sequential rounds of antigen enrichment during DMS studies. (A) Libraries designed from DMS data following one round of antigen enrichment (Ag+, FIG. 10A). (B) Libraries designed from DMS data following two rounds of antigen enrichment (Ag+2, FIG. 10B). (C) Libraries designed from DMS data following three rounds of antigen enrichment (Ag+3, FIG. 10C).

FIG. 12 illustrates the exemplary next-generation sequencing results for sequence reads, alignment, and number of unique sequences detected for NGS performed on the library (Ab+), non-binding variants (Ag−), and binding variants after 1 and 2 rounds of enrichment (Ag+1, Ag+2).

FIGS. 13A and 13B illustrate the exemplary next-generation sequencing results for sequence reads, alignment, and number of unique sequences detected for NGS performed on the combinatorial mutagenesis libraries.

FIGS. 14A and 14B illustrate exemplary flow cytometry data for Trastuzumab (Herceptin) CDRH3 DMS-based combinatorial mutagenesis libraries. Following transfection and integration of the DMS-based combinatorial mutagenesis library, the frequency of antigen-specific variants can be used to assist in model performance and evaluation. In the example provided, approximately 10% of antibody variants are antigen-specific. FIG. 14A discloses SEQ ID NOS 27-28, respectively, in order of appearance.

FIG. 15A to FIG. 15D illustrate experimental validation data for 104 variants obtained by in silico selection. FIGS. 15A-15D disclose SEQ ID NOS 29-53, 53, 54-79, 80-84, 68, 85-104, 105-109, 103, 110-118, 116, and 119-128, respectively, in order of appearance.

FIGS. 16A-D illustrate experimental validation data for antibody sequences predicted according to the methods disclosed therein. FIG. 16A depicts protein expression levels for various predicted antibody sequences as compared to expression levels of trastuzumab (farthest right). FIG. 16B depicts binding kinetics of the predicted antibody sequences. The binding kinetics of trastuzumab is indicated in the nanomolar range. FIG. 16C depicts thermal stability of the predicted antibody sequences as compared to thermal stability of trastuzumab (farthest right). FIG. 16D depicts immunogenicity risk of two predicted sequences (C and F) as compared to trastuzumab.

FIGS. 17A-21B illustrate model performance curves for classification of binders and non-binders on unseen test data. 30% of the initial data set was split into two test data sets (15% each). One test data set contains the same ratio of binding and non-binding sequences present in the training data set (TEST SET A) and the other test data set contains an approximate ratio of 10/90 binding and non-binding sequences (TEST SET B) to resemble physiological frequencies observed in the data illustrated in FIGS. 14A-B. (Top panels) ROC (receiver operating character) curve and PR (precision-recall) curve observed on the classification of sequences in TEST SET A; (Bottom panels) ROC curve and PR curve observed on the classification of sequence in TEST SET B; (A) LSTM-RNN (Long-short term memory recurrent neural network) ROC curves (left panels), LSTM-RNN PR curves (right panels); (B) CNN (convolutional neural network) ROC curves (left panels), CNN PR curves (right panels)

FIG. 22 provides a summary of the AUC (area under the curve), average PR and the number of predicted binders for each of the model performance curves shown in FIGS. 17-21.

FIGS. 23A-230 illustrate exemplary data for the flow cytometry analysis (left) and biolayer interferometry affinity analysis (right) for the tested variants. FIGS. 23A-230 disclose SEQ ID NOS 74, 58, 78, 56, 30, 113, 73, 64, 119, 44, 51, 127, 37, 50, 39, 62, 98, 96, 114, 110, 36, 106, 79, 48, 111, 65, 89, 83, 82, and 88, respectively, in order of appearance.

FIG. 24A illustrates a table of flow cytometry labeling conditions for deep mutational scanning studies.

FIG. 24B illustrates flow cytometry labeling conditions for DMS-guided combinatorial mutagenesis libraries.

FIG. 25 illustrates exemplary flow cytometry data for Trastuzumab (Herceptin) CDRL3 deep mutation scanning. (A) Flow cytometry plot, heatmap, and sequence logo plot following FACS for antibody expressing (Ab+) cells and antigen-specific (Ag+) cells. (B) Flow cytometry plot, heatmap, and sequence logo plot following a second round of enrichment for antigen-specific (Ag+2) cells; Decreased antigen concentration was used for flow cytometry labeling. (C) Flow cytometry plot, heatmap, and sequence logo plot following a third round of enrichment for antigen-specific (Ag+3) cells; Labeling for flow cytometry performed with antigen containing an alternatively conjugated fluorophore (Alexa Fluor 488). All enrichment ratios (ER) are calculated by dividing the frequency of a mutant found in the respective Ag+ population by the frequency of the mutant found in the Ab+ population.

FIG. 26 illustrates exemplary next-generation sequencing results for sequence reads, alignment, and number of unique sequences detected from NGS performed on the CDRL3 library (Ab+) and binding variants after 1 and 2 rounds of enrichment (Ag+1, Ag+2).

FIG. 27 illustrates exemplary workflow and flow cytometry data for generating antigen specific libraries in mammalian cells at multiple locations along the antibody (e.g. CDRL3 and CDRH3). Initial libraries are generated by transfecting gRNA and ssODN donor templates containing rationally designed libraries for the first region. Antibody expressing cells (Ab+) are enriched by fluorescence activated cell sorting (FACS). Libraries in the second region are then generated by transfecting gRNA and ssODN donor templates containing rationally designed libraries for the second region. Antibody expressing cells (Ab+) are enriched by fluorescence activated cell sorting (FACS). Ab+ cells can then undergo multiple rounds of enrichment for antigen-specific variants. Antigen-specific libraries are designed from enrichment ratios calculated following sequential rounds of antigen enrichment during DMS studies. (A) CDRL3 libraries designed from DMS data following two rounds of antigen enrichment (Ag+2, FIG. 25C). (B) CDRH3 libraries designed from DMS data following two rounds of antigen enrichment (Ag+3, FIG. 10C). (C-D) Experimental results from sanger sequencing experiments derived from the final CDRL3+CDRH3 mutagenesis library validating genetic diversity introduced into both regions. (E) illustrates exemplary workflow and flow cytometry data for generating antigen specific libraries first at CDRL3 and then at CDRH3. FIG. 27C discloses SEQ ID NOS 129-131, respectively, in order of appearance and FIG. 27D discloses SEQ ID NOS 132-134, respectively, in order of appearance.

FIG. 28 illustrates exemplary data for Adalimumab (Humira) CDRH3 deep mutation scanning. Heatmap and sequence logo plot generated from deep sequencing of libraries following FACS for antibody expressing (Ab+) cells and antigen-specific (Ag+) cells; Labeling for flow cytometry performed with antigen containing an alternatively conjugated fluorophore (Alexa Fluor 488).

FIG. 29 illustrates exemplary next-generation sequencing results for sequence reads, alignment, and number of unique sequences detected from NGS performed on the adalimumab CDRH3 library (Ab+) and binding variants after 1 and 2 rounds of enrichment (Ag+1, Ag+2).

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

Phage and yeast display screening are useful for high-throughput screening of large mutagenesis libraries (>10⁹), however they are primarily used for only increasing affinity or specificity to the target antigen. Nearly all therapeutic antibodies can require expression in mammalian cells as full-length IgG, which means that the development and optimization steps following initial selection must occur in this context. Since mammalian cells lack the capability to stably replicate plasmids, this last stage of development is done at very low-throughput, as elaborate cloning, transfection and purification strategies must be implemented to screen libraries in the max range of 10³ antibodies. Thus, only minor changes (e.g., point mutations) are screened at this stage, typically resulting in only a few optimized leads. Interrogating such a small fraction of protein sequence space also implies that addressing one development issue will frequently cause rise of another or even diminish antigen binding altogether, making multi-parameter optimization very challenging.

The methods described herein include an improved therapeutic antibody development process that employs an effective combination of directed evolution from rationally designed mutagenesis libraries with machine learning. Deep learning models to interrogate and predict antigen-specificity from a massive diversity of antibody sequence space enables the generation of thousands of optimized lead candidates.

In some aspects, a mammalian display platform is used, where rationally designed site-directed mutagenesis libraries are introduced using high throughput mutagenesis systems for mammalian expression, such as by CRISPR/Cas9-mediated homology-directed repair (HDR). The inventors have found that screening and deep sequencing of relatively small libraries (e.g., about 10⁴) generated based on the described methods, produced high quality data capable of training deep neural networks that predict antigen-binding based on antibody sequence with over 80% precision.

Once trained according to the methods described herein, machine learning models can then be used to predict millions of antigen binders from a much larger in silico generated library variants (e.g., ˜10⁸ variants were generated by the methods described herein when trastuzumab was used as an input amino acid sequence). These variants can be subjected to multiple developability filters, resulting in tens of thousands of optimized lead candidates. As described herein in the Examples, when the present methods were applied to the heavy chain complementarity determining region 3 (CDRH3) of an exemplary antibody, the therapeutic antibody Trastuzumab, it was observed that of the small subset of only 30 optimized lead candidates that were expressed and assayed for antigen binding, 29 were shown to be antigen-specific. Thus, nearly all optimized lead candidates that were selected for testing possessed the predicted property. With its scalable throughput and capacity to interrogate across a vast protein sequence space, the methods described herein can be applied to a wide variety of applications that involve the engineering and optimization of antibody and other protein-based therapeutics.

The present disclosure describes systems and methods to make predictions of protein sequence-phenotype relationships and can be employed for the identification of therapeutic antibodies with one or more desired parameters, such as antigen specificity or affinity. The system can include one or more machine learning models that can extrapolate complex relationships between protein sequence and function. In some aspects, the models can be trained on high-quality training data generated through a two-step directed evolution approach, that combines single-site mutagenesis scanning followed by a combinatorial deep mutational scanning approach. The trained models described herein can then make predictions regarding new antibody sequences generated in silico. The systems and methods described herein enable the interrogation of a much larger sequence space than what is physically possible with standard expression systems, such as phage or bacterial display. For example, for a short stretch of 10 amino acids, the combinatorial sequence diversity explodes to 10¹³, a size which is nearly impossible to interrogate experimentally. In some aspects, the systems described herein can also perform multi-parameter optimization to identify, from the variants classified by the models as antigen-binders, the antigen-binder classified variants that are most likely to exhibit antigen-specificity.

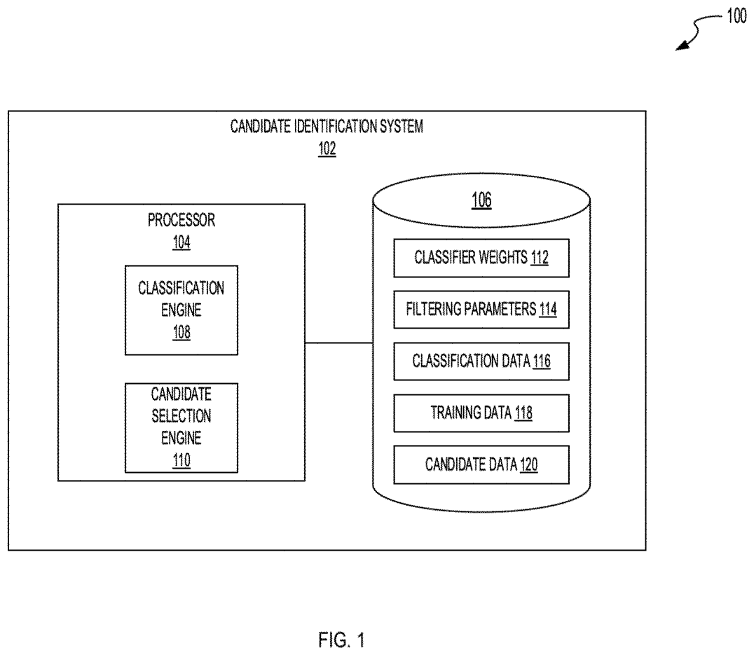

FIG. 1 illustrates a block diagram of an example system 100 to select antibody lead candidates. The candidate identification system 102 can include one or more processors 104 and one or more memories 106. The processors 104 can execute processor-executable instructions to perform the functions described herein. The processor 104 can execute a classification engine 108 and a candidate selection engine 110. The memory 106 can store processor-executable instructions, generate data, and collected data. The memory 106 can store one or more classifier weights 112 and filtering parameters 114. The memory 106 can also store classification data 116, training data 118, and candidate data 120.

The system 100 can include one or more candidate identification systems 102. The candidate identification system 102 can include at least one logic device, such as the processors 104. The candidate identification system 102 can include at least one memory element 106, which can store data and processor-executable instructions. The candidate identification system 102 can include a plurality of computing resources or servers located in at least one data center. The candidate identification system 102 can include multiple, logically-grouped servers and facilitate distributed computing techniques. The logical group of servers may be referred to as a data center, server farm, or a machine farm. The servers can also be geographically dispersed. The candidate identification system 102 can be any computing device. For example, the candidate identification system 102 can be or can include one or more laptops, desktops, tablets, smartphones, portable computers, or any combination thereof.

The candidate identification system 102 can include one or more processors 104. The processor 104 can provide information processing capabilities to the candidate identification system 102. The processor 104 can include one or more of digital processors, analog processors, digital circuits to process information, an analog circuit designed to process information, a state machine, and/or other mechanisms for electronically processing information. Each processor 104 can include a plurality of processing units or processing cores. The processor 104 can be electrically coupled with the memory 106 and can execute the classification engine 108 and the candidate selection engine 110.

The processor 104 can include one or more microprocessors, application-specific integrated circuits (ASIC), field-programmable gate arrays (FPGA), or combinations thereof. The processor 104 can be an analog processor and can include one or more resistive networks. The resistive network can include a plurality of inputs and a plurality of outputs. Each of the plurality of inputs and each of the plurality of outputs can be coupled with nanowires. The nanowires of the inputs can be coupled with the nanowires of the outputs via memory elements. The memory elements can include ReRAM, memristors, or PCM. The processor 104, as an analog processor, can use analog signals to perform matrix-vector multiplication.

The candidate identification system 102 can include one or more classification engines 108. The classification engine 108 can include one or more machine learning algorithms configured to extract features from data and classify the data based on the extracted features. For example, the classification engine 108 can include one or more of a recurrent neural network (e.g., a type of artificial neural network derived from feedforward neural networks in which connections between nodes form a directed graph along a temporal sequence to allow for temporal dynamic behavior), a convolutional neural network (e.g., a neural network with layers of nodes that are connected to one-another and use convolution in at least one of the layers), a standard artificial neural network (e.g., a computing system based on a collection of connected units or nodes configured to learn to perform tasks based on examples or training data), a support vector machine (e.g., a supervised learning model with associated learning functions that analyze data used for classification and regression analysis), a random forest ensemble (e.g., a computing system learning method for classification, regression and other tasks that operate by constructing a multitude of decision trees at training time and outputting the class is the mode of the classes or mean prediction of the individual trees) or a logistic regression model (e.g., a statistical technique that can use a logistic function to model the probability of a certain class or event existing such as a binary dependent variable).

For example, the classification engine 108 can include an artificial neural network. The neural network can include an input layer, a plurality of hidden layers, and an output layer. The neural network can be a multi-layered neural network, a convolution neural network, or a recurrent neural network, including a long-short-term-memory (LSTM) neural network. The classification engine 108 can include a plurality of neural networks or classification models. For example, the classification engine 108 can process the classification data 116 with a first classification model (e.g., a convolution neural network) and also with a second classification model (e.g., an LSTM neural network). As described below in relation to the candidate selection engine 110, the candidate selection engine 110 can select candidate antibodies as the antibodies that were identified by the first and second classification model.

During a training phase, the classification engine 108 can process training data 118 to generate the weights and biases for one or more of the classification engine's machine learning models. Once trained, the classification engine 108 can store the weights and biases as the classifier weights 112 in the memory 106. The generation of the training data and training of the classification engine 108 is described further in relation to the memory 106, training data 118, and examples, below.

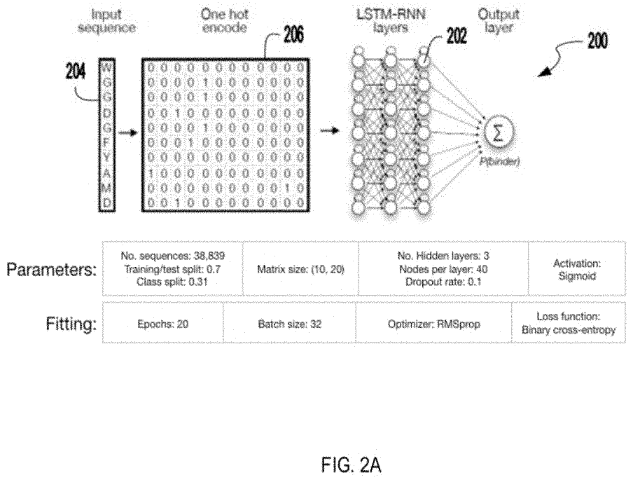

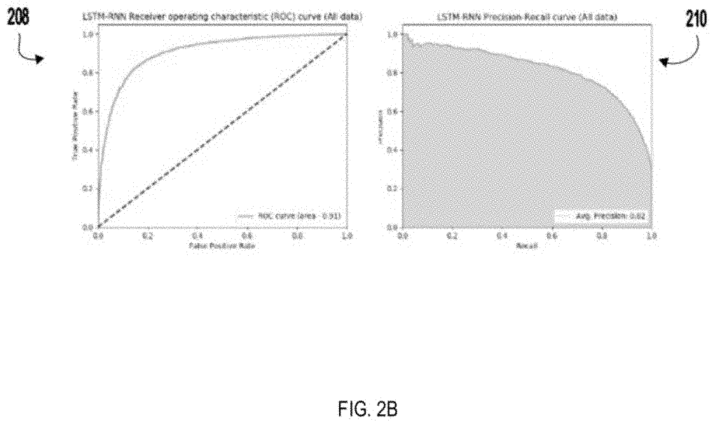

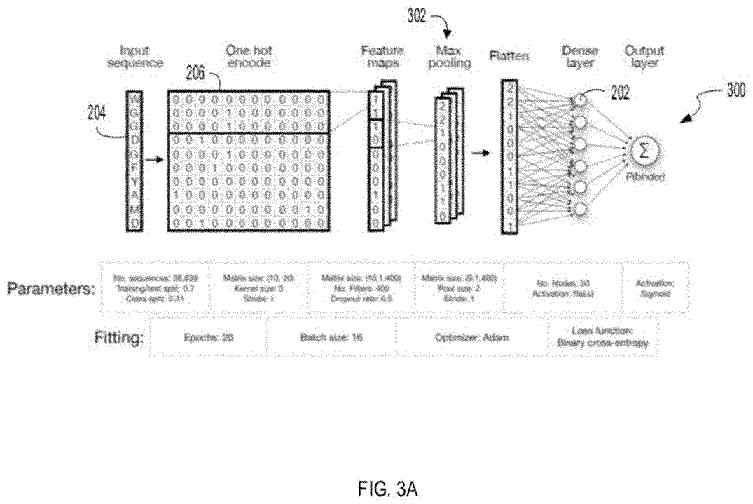

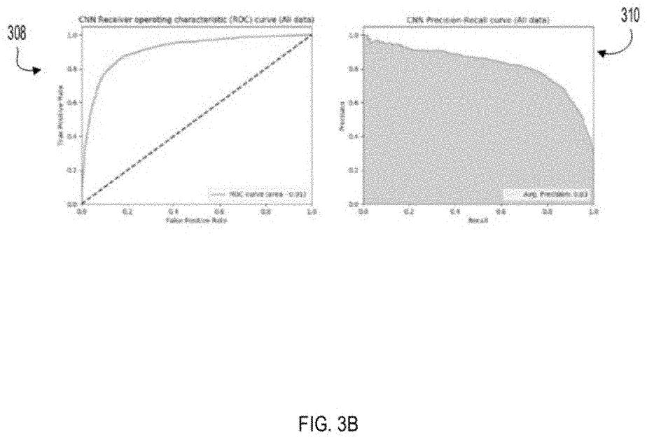

The classification engine 108 can generate the weights and biases by inputting the training data 118 into the neural network and comparing the resulting classification to the expected classification (as defined by the input data's label). For example, in an example system that includes 10 output neurons that each correspond to a different classification, the classification engine 108 can use back-propagation and gradient descent to minimize the cost or error between the expected result and result determined by the classification engine 108. Once the classification engine 108 has trained its neural network, the classification engine 108 can save the weights and biases to the memory 106 as classifier weights 112. The models (e.g., the convolution neural network and the LSTM neural network) of the classification engine 108 are described further in relation to FIGS. 2 and 3, among others.

The candidate identification system 102 can include a candidate selection engine 110. For a given protein sequence space (e.g., all possible protein sequence variants), the classification engine 108 can classify a large number of the variants as antigen-binders. The candidate selection engine 110 can select candidate variants from the variants classified as antigen-binders for further testing or study. The candidate selection engine 110 can select the candidate variants by applying one or more filtering policies to the antigen-binder classified variants. The filtering policies can include one or more filtering parameters 114, each with an associated threshold or other constraint. The candidate selection engine 110 can select the antigen-binder classified variants as candidate variants if the antigen-binder classified variant satisfies the, for example, threshold of the respective filtering parameters 114.

The candidate selection engine 110 can select an antigen-binder classified variant as a candidate variant if more than one model of the classification engine 108 classifies the variant as an antigen-binder. For example, the classification engine 108 can include a convolution neural network and an LSTM neural network. The classification engine 108 can classify each of the variants in the variant space with the convolution neural network and the LSTM neural network to generate two classifications for each variant (e.g., one classification by the convolution neural network and a second classification by the LSTM neural network). When the classification engine 108 performs classification with multiple models to generate multiple classifications for each variant. A consensus between the models can be one of the filtering parameters 114. For example, variants not classified as antigen-binder classified variants by both the convolution neural network and the LSTM neural network can be discarded from further processing. The candidate data 120 can include variants that are classified as antigen-binder classified variants by both the convolution neural network and the LSTM neural network.

The filtering parameters 114 can include a similarity metric requirement to a known wild-type antibody sequence. For example, the candidate selection engine 110 can calculate a Levenshtein distance between each variant in the variant space and the known wild-type sequence to determine a similarity between the respective variant and the wild-type sequence. The filtering policy can indicate that each candidate variant must satisfy a similarity threshold with the wild-type sequence. For example, the candidate selection engine 110 can select antigen-binder classified variants as candidate variants for storage in the candidate data 120 if the antigen-binder classified variants have a Levenshtein distance less than 5, for example. The candidate selection engine 110 can select antigen-binder classified variants that have a Levenshtein distance greater than 5 in some examples.

The filtering parameters 114 can include a similarity metric to human antibody repertoire sequences. For example, the candidate selection engine 110 can calculate a Levenshtein distance between each variant in the variant space to a collection of human antibody sequences (e.g., from patient B cells) to determine a similarity between the respective variant and the human repertoire. Based on filtering policy, the candidate selection engine 110 can select candidate variants that satisfy a similarity threshold to human repertoire sequences.

The filtering parameters 114 can include any developability attribute of a protein, including, for example, a net charge, hydrophobicity index, viscosity, clearance threshold, solubility, affinity, chemical stability, thermal stability, expressability, specificity, cross-reactivity, or any combination thereof. The candidate selection engine 110 can calculate, for each antigen-binder classified variant, the net change and the hydrophobicity of the antigen-binder classified variant. Based on the net charge and the hydrophobicity, the candidate selection engine 110 can calculate a viscosity value and clearance value for the antigen-binder classified variant. For example, viscosity can decrease with increasing variable fragment (Fv) net charge and increasing Fv charge symmetry parameter (FvCSP). The filtering parameters 114 can include a clearance value based on the variable fragment (Fv) charge between about 0 and about 6.2 with a CDRL1+CDRL3+CDRH3 hydrophobicity index sum less than 4.0. The candidate selection engine 110 can identify protein sequence motifs associated with manufacturing liabilities, such as n-glycosylation sites, deamidation sites, isomerization sites, methionine oxidation, tryptophan oxidation and paired or unpaired cysteine residues. For example, the candidate selection engine 110 can select antigen-binder classified variants with zero sequence motifs associated with manufacturing liabilities. The candidate selection engine 110 can include a protein solubility predictor to predict a protein solubility for each of the antigen-binder classified variants. For example, the candidate selection engine 110 can select antigen-binder classified variants with a solubility greater than 1 as candidate variants. In some implementations, the candidate selection engine 110 can select the antigen-binder classified variants with a solubility or other developability attribute above a threshold. The threshold can be a value threshold. The threshold can be a variable or relative threshold. For example, the threshold can be the top 5%, 10%, or other percentage of the antigen-binder classified variants. In another example, the candidate selection engine 110 can select antigen-binder classified variants above a number of standard deviations above the average.

The candidate selection engine 110 can calculate an affinity binding score for each of the antigen-binder classified variants for MHC Class II molecules in order to filter out candidate peptides that may be immunogenic. For example, the candidate selection engine 110 can predict the peptide binding affinity of the variant sequences to MHC Class II molecules by utilizing a tool, such as NetMHCIIpan, which predicts binding of peptides to the three human MEW class II isotypes HLA-DR, HLA-DP and HLA-DQ. The CDRH3 sequences can be padded with 10 amino acids on the 5′ and 3′ ends and then all possible 15-mers can be run through NetMHCllpan. The candidate selection engine 110 can determine an antigen-binder classified variant's percentage rank predicted affinity for MHC Class II compared to a set of 200,000 random natural peptides. The candidate selection engine 110 can filter out antigen-binder classified variants with a percentage rank less than about 20%, 15%, 10%, 5%, or 2%. The lower the percentage rank, the higher the predicted affinity of the antigen-binder classified variant for MHC Class II. In some aspects, sequences can be filtered out if any of the 15-mers contain a % Rank <15. The average % Rank across all 15-mers for the remaining sequences can further be calculated and those with an average % Rank <70 can be filtered out. The mean and median values for the predicted binding affinity can further be calculated across all MEW class II alleles for each of the 15-mers and those sequences with a mean and/or median greater than a defined threshold can be filtered out. The filtering policy can indicate that an antigen-binder classified variant must satisfy one or more of the filtering parameters 114 to be selected as a candidate variant and be stored as candidate data 120.

The candidate identification system 102 can include one or more memories 106. The memory 106 can be or can include a memory element. The memory 106 can store machine instructions that, when executed by the processor 104 can cause the processor 104 to perform one or more of the operations described herein. The memory 106 can include but is not limited to, electronic, optical, magnetic, or any other storage devices capable of providing the processor 104 with instructions. The memory 106 can include a floppy disk, CD-ROM, DVD, magnetic disk, memory chip, ROM, RAM, EEPROM, EPROM, flash memory, optical media, or any other suitable memory from which the processor 104 can read instructions. The instructions can include code from any suitable computer programming language such as, but not limited to, C, C++, C#, Java, JavaScript, Perl, HTML, XML, Python, and Visual Basic.

The candidate identification system 102 can store classifier weights 112 in the memory 106. The classifier weights 112 can be a data structure that includes the weights and biases that define the neural networks of the classification engine 108. Once trained, the classification engine 108 can store the classifier weights 112 to the memory 106 for later retrieval and use in classifying classification data 116.

The candidate data 120 can store filtering parameters 114 in the memory 106. As described above, the candidate selection engine 110 can retrieve a filtering policy for selecting candidate variants from the antigen-binder classified variants. The candidate selection engine 110 can apply the filtering policy to identify antigen-binder classified variants that have a higher likelihood of having a relatively high affinity for a given antigen. The filtering parameters 114 can each be a data structure that indicates a threshold value for the respective filtering parameter 114. For example, a filtering parameter can indicate that antibody for a given antigen-binder classified variant should have a Fv net charge between about 0 and about 6. Each filtering parameter 114 can indicate a specific parameter and predetermined threshold (e.g., above 2), a predetermined range (e.g., between 0 and 6), an adaptive threshold (e.g., having a predicted affinity within the top 5% of the antigen-binder classified variants), or an adaptive range (e.g., between about the top 1% and 5% of predicted affinities for the antigen-binder classified variants).

The candidate identification system 102 can store classification data 116 in the memory 106. The classification data 116 can be a plurality of variants that are to be classified by the classification engine 108. The classification data 116 can include each variant in the variant space for a given sequence. For example, the candidate identification system 102 can start with a predetermined antibody and calculate all possible variants of the antibody. Each of the variants can be stored in the memory 106 as classification data 116.

The candidate identification system 102 can store training data 118 in the memory 106. The training data 118 can include a data structure that includes indications of a plurality of variants. Each variant of the training data 118 can be stored separately (e.g., as a single string or vector) or collectively (e.g., as a matrix where each column or row corresponds to a different variant). The training data can be labeled training data 118 to indicate whether the respective variant is a binding or non-binding variant. For example, each variant can be stored as a binary file encoding the sequence of the variant. The binary file can include a leading (or trailing) bit that can be set (e.g. set to 1) to indicate that the variant is a binding variant or not set (e.g., set to 0) to indicate that the variant is a non-binding variant.

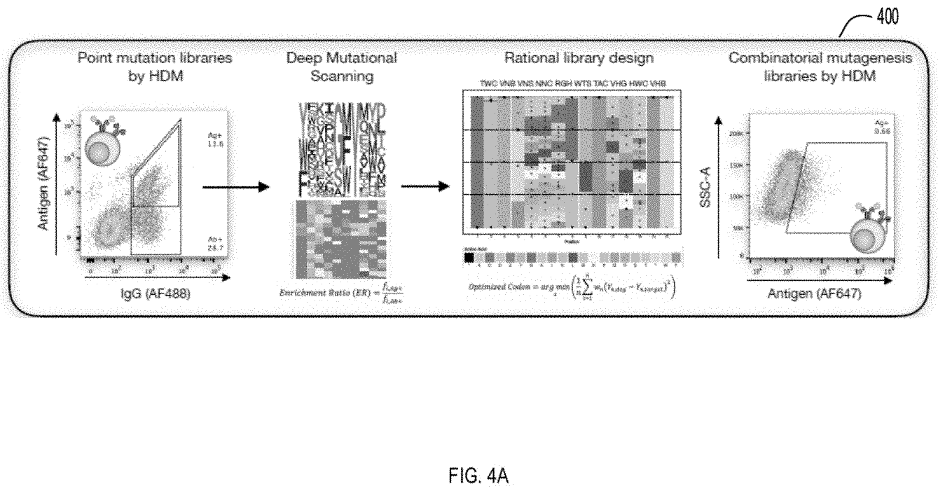

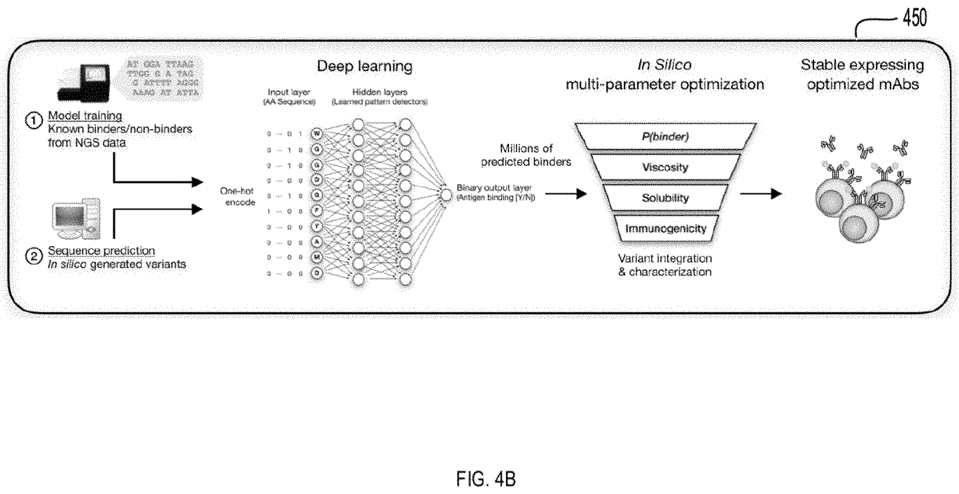

The training data 118 can be a set of variants that is selected by physical screening of a rationally designed library of variants based on a selected parameter (e.g., antigen binding). For example, in some embodiments, the training data includes numerical values. In some embodiments, the numerical values correspond to binding kinetic values for a set of variants. In some embodiments, the numerical values correspond to numerical value results for biophysical assays (e.g., melting temperature for thermal stability, or AC-SINS for solubility). Exemplary methods for generation of the training data is described in further detail (see, e.g., FIG. 4A).

The classification engine 108 can be trained using the training data 118. The classification engine 108 can be trained, in this example, to predict specificity towards a target antigen. As described further, below, in relation to FIGS. 2 and 3, the training data 118 (like the classification data 116) can be one-hot encoded for input into the classification engine 108. The training data 118 can be divided into training data and testing data. For example, the training data can be used to train the classification engine 108 and the testing data can be reserved to test the accuracy and precision of the trained classification engine 108 instead for the training of the classification engine 108. The testing data can be labeled to enable the classification engine 108 to determine whether the variants of the testing data was properly classified. In one example, 70% of the training data 118 can be set aside for training and 30% can be used for testing or evaluation of the classification engine 108. The testing data can be split to include predetermined proportions of binder to non-binder variants. For example, the testing data can be split to approximately 10/90 binders/non-binders to resemble physiological frequencies.

The candidate selection engine 110 can store candidate variants in the memory 106 as candidate data 120. The candidate data 120 can be a data structure that can indicate each of the antigen-binder classified variants that satisfy the parameters of the filtering policy. The candidate data 120 can be a data structure that can indicate each variant classified as an antigen-binder before or without processing the antigen-binder classified variants with the filtering policy. The data structure can be a text-based file or a binary file that indicates the sequence of the variant. For example, the sequence can be stored as a character string in a text-based file. The data structure (or file) can include metadata such as which positions were mutated with respect to wild-type and the nature of the mutation. The metadata can include a classification score that indicates the certainty with which the classification engine 108 classified the antigen-binder classified variant as an antigen-binder classified variant.

FIG. 2 illustrates an example neural network 200. The neural network 200 can be an LSTM neural network 200. See FIG. 2A. The LSTM neural network 200 can include a plurality of nodes 202, which can also be referred to as neurons 202. The nodes 202 can be arranged in layers. For example, the node 202 can include an input layer of nodes 202, one or more hidden layers of nodes 202, and an output layer of nodes 202. Each of the layers can include one or more nodes 202. For example, the input layer can include 10 nodes 202 (e.g., the number of nodes 202 in the input layer is equal to the length of the input vector 204) and the output layer can include one node 202. The node 202 of the output layer can indicate the probability that the input vector 204 corresponds to an antigen-binder classified variant. The LSTM neural network 200 can include two output nodes 202—one node 202 that provides the probability that the variant is an antigen-binder classified variant and a second node 202 that provides the probability that variant is a non-antigen-binder classified variant.

The LSTM neural network 200 can include between about 2 and about 10, between about 2 and about 8, between about 2 and about 6, between about 2 and about 4, or between about 2 and about 3 layers. Each layer can include the same number of nodes 202 or a different number of nodes 202. The input layer can include a node 202 for each value on a one-hot encoded matrix input. For example, for a 10×20 one-hot encoded matrix, the input layer can include 200 nodes 202. The number of nodes 202 in the input layer can be based on the number of values in the input sequence (e.g., the number of amino acids in the sequence) times the number of possible values for each value. For example, for a sequence of length 10 with 20 possible amino acids per position, the input layer can include 10×20=200 nodes 202. The LSTM neural network 200 can include a plurality of hidden layers. Each of the hidden layers can include the same or a different number of nodes 202. The hidden layers can include fewer nodes 202 than the input layer. For example, the hidden layers can each include 40 nodes 202.

Each node 202 in a layer can be linked to each node 202 in a subsequent layer. Each node 202 outputs, to the nodes 202 to which it is connected, a weighted sum of the node's inputs. The node 202 can add a bias to the weighted sum to bias the output. The node 202 can include an activation function (e.g., a sigmoid function, a rectified linear unit (ReLU), or leaky rectified linear unit) that determines when the node 202 “fires” or outputs a signal based on the weighted sum. The weights of each link and the bias for each node 202 can be set during the training phase and stored as classifier weights 112. The LSTM neural network 200 can be a recurrent neural network, and each node 202 can provide feedback (or input) to itself. The recurrent neural network can create an internal state to exhibit temporal behaviors.

To classify a variant, the classification engine 108 converts the sequence of the variant into an input vector 204, where each value of the input vector 204 corresponds to a respective amino acid of the sequence. The input vector 204 has a length equal to the length of the input sequence. The classification engine 108 can one-hot encode the input vector 204 to generate a matrix 206. The input vector 204 can include other features of the variant sequence. For example, biophysical properties of the variant sequence can be encoded into the input vector 204. Each row of the matrix 206 corresponds to a respective value (e.g., position) of the input vector 204. Each column of the matrix 206 corresponds to a different possible amino acid that can fill each respective value of the input vector 204. In this example, as there are twenty amino acids, the matrix 206 includes twenty columns. Each row of the matrix 206 includes a 1 in the column corresponding to the amino acid present in the respective value of the input vector 204. The matrix 206 can be flattened to a vector and each value from the vector can be provided to one of the nodes 202 of the input layer. The matrix 206 can be sequentially provided to the nodes 202 of the input layer. For example, the input layer can include 10 input nodes 202 and the columns (e.g., the 10 values of each column) of the matrix 206 can be sequentially provided to the input nodes 202.

To classify a variant, the classification engine 108 can convert the sequence of the variant into an input vector 204, where each value of the input vector 204 corresponds to a respective amino acid of the sequence. The input vector 204 has a length equal to the length of the input sequence. Encoding of the input vector can also take place based on protein physical properties, as each individual amino acid is represented with a collection of physical properties (e.g., charge, hydrophobicity, volume).

FIG. 2B illustrates the receiver operating characteristic (ROC) curve 208 for the LSTM neural network 200 on a test data set and the precision-recall (PR) curve 210 for the LSTM neural network 200. The ROC curve 208 and the PR curve 210 indicate the accuracy of the LSTM neural network 200. The curves 208 and 210 were generated by providing the LSTM neural network 200 a test data set of unseen variants at a 50/50 split of binders to non-binders.

FIG. 3 illustrates an example neural network 300. The neural network 300 can be a convolutional neural network 300. See FIG. 3A The convolutional neural network 300 can include a plurality of nodes 202. The convolutional neural network 300 can include a plurality of layers 302. Unlike the neural network 200, each of the layers 302 in the convolutional neural network 300 may not be fully connected. For example, a node 202 of a given layer 302 may not be connected to each node 202 in a subsequent layer 302. The convolutional neural network 300 can include a plurality of filters. The convolutional neural network 300 can convolve the matrix 206 with each of the plurality of filters to generate a plurality of feature maps. Each filter can be configured to detect predetermined patterns in the matrix 206. The filter can be 1D convolutional filters with a dilation rate of and a stride size of 1 with a kernel size of 3, which can result in a filter of size 20×3. The convolution neural network 300 can include between about 100 and about 400 filters. The numbers of filters can be selected by cross-validation, or splitting the data into train/validation/test sets and choosing the optimal configuration via a random/grid-search. The convolutional neural network 300 can include one or more max pooling layers to reduce the spatial size of the feature maps. The convolutional neural network 300 can include a flattening layer that flattens the max pooled layer into an input vector for a fully connected layer of nodes. Each value in the flattened layer can act as an input to each of the nodes 202 in the dense (or fully connected) layer. The convolutional neural network 300 can include 50 nodes 202 in the dense layer. The number of nodes can be selected based on a limited cross-validation/grid-search procedure. As with the LSTM neural network 200, each node 202 in the dense layer can serve as an input to an output node 202.

FIG. 3B illustrates the ROC curve 308 for the convolutional neural network 300 on a test data set and the PR curve 310 for the convolutional neural network 300. The ROC curve 308 and the PR curve 310 indicate the accuracy of the convolutional neural network 300. The curves 308 and 310 were generated by providing the convolutional neural network 300 unseen variants at a 50/50 split of binders to non-binders.

Referring to FIGS. 2 and 3, among others, the LSTM neural network 200 and convolutional neural network 300 architecture and hyper-parameters were selected by performing a grid search across various parameters. For example, the LSTM neural network 200 the grid search was performed to determine the nodes 202 per layer, the batch size, the number epochs, and optimizing function. For the convolutional neural network 300, classification engine 108 determines the number of filters, the kernel size, the dropout rate, the number of nodes 202 in the dense layer nodes based on a k-fold cross-validation of the data set.

FIG. 4A illustrates a flow process 400 for generating the training data 118. The training data 118 can be a set of variants that is selected by physical screening of a rationally designed library of variants based on a selected parameter (e.g., antigen binding). The flow process 400 can include generating a point mutation library using, for example, homology-directed mutagenesis (HDM) or any other suitable mutagenesis method. In some aspects, the set of variants is selected in a two-step screening process that includes single-site (i.e. point mutation) and combinatorial deep mutational scanning (DMS) processes, an example of which is illustrated in flow process 400. The amino acid sequence of an antibody's heavy chain complementarity determining region 3 (CDRH3) is a key determinant of antigen specificity. Thus, two-step DMS process can be performed on this selected region (e.g., 10 amino acids of the CDRH3) to resolve the specificity determining amino acid positions. In some aspects, a mutant full-length antibody that has a variant CDRH3 sequence (e.g., a mutated CDRH3 sequence) such that the antibody no longer binds to its antigen can be used as a starting sequence. Starting with mutant non-binding variant can provide advantages in the selection of binders from the library by reducing the background from the original sequence. In some alternate implementations, the process can start with a variant that still binds to its antigen.

While FIG. 4A exemplifies training data for the CDRH3 of an antibody, the methods described herein are not so limited and can be applied to a set of variants for one or more regions of interest in an antibody or other binding protein, such as a receptor that binds to a ligand. For example, the set of variants can represent other CDR regions of an antibody, such as CDRH1, CDRH2, CDRL1, CDRL2, CDRL3, combinations of two or more CDR regions, a region within the framework domains of the antibody (e.g., FR1, FR2, FR3, FR4), or regions within the constant domains of the antibody (e.g., CH1, CH2, CH3) for which improvement of one or more properties of the antibody is desired. In some aspects, the variant is a full-length antibody. In some aspects, the variant is a fragment of an antibody of a recombinant antibody comprising an antigen binding domain, such as an scFv or an Fc fusion protein. In some aspects, the training data is derived from variants of a binding protein, such as a receptor, that binds to a ligand.

In the first step of the exemplary flow process 400, a mutagenesis method is applied to the CDRH3 sequence to generate a library of variants as single sites at each position of the CDRH3 sequence (referred to herein as single-site DMS). Any suitable method of producing single point mutations can be employed. In some aspects, a hybridoma cell line expressing a full-length antibody variant sequence is used. Libraries of variant antibody sequences can be generated by CRISPR-Cas9-mediated homology-directed mutagenesis (HDM) (See, e.g., PCT Publication No. WO 2017/174329, which is incorporated by reference in its entirety). For example, gRNA for Cas9 targeting of CDRH3 and a pool of homology templates in the form of single-stranded oligonucleotides (ssODNs) containing NNK degenerate codons at single amino acid positions across the CDRH3 can be used to introduce point mutations at single sites in the CDRH3 of the antibody. Alternatively, any suitable mutagenesis method can be used to generate variants, for example, error-prone PCR, recombination mutagenesis, alanine scanning mutagenesis, structure-guided mutagenesis. In some aspects, the mutagenesis can be performed on the nucleic acid sequence encoding the amino acid sequence of interest using in vitro techniques (e.g. PCR) and then the variant nucleic acids introduced into mammalian cells (e.g., by CRISPR-Cas9 HDR).

Libraries of cells expressing the variant full-length antibodies can then be screened by a suitable method to detect antigen binding, such as by fluorescence-activated cell sorting (FACS). Exemplary FACS results for the first step of the screening process are shown in the first step of process 400. Populations of cells expressing the antibody and selected for binding or not binding antigen can then be subjected to deep sequencing to determine the antibody sequences expressed by the selected cells.

The flow process 400 can include deep mutational scanning to determine enrichment scores for each amino acid position assayed to determine which positions are more or less amenable to accepting mutations. For example, the variant libraries were screened by FACS, and populations expressing antibody and binding or not binding antigen were subjected to deep sequencing. In some aspects, populations of cells that bind to two or more antigens are selected (e.g. cross-reactive or multispecific antibodies). The enrichment scores, which can be referred to as enrichment ratios (ER), can be the ratio of clonal frequencies of variants enriched for antigen specificity by FACS,fᵢ,Ag₊, to the clonal frequencies of the variants present in the original library, fᵢ,Ab₊. More particularly:

$$E R = \frac{{f}_{i} , {Ag}_{+}}{{f}_{i} , {Ab}_{+}} \qquad ( 1 )$$

In some implementations, a minimum value of −2 was designated to variants with log[ER] values less than or equal −2 and variants not present in the dataset were disregarded in the calculation. A clone was defined based on the specific amino acid sequence of the CDRH3. Heatmaps and their corresponding sequence logo plots can then be generated based on the enrichment scores from the first step of the screening process. The heatmaps and sequence logo plots can then be used to rationally design a combinatorial mutagenesis library for screening. Degenerate codons can be selected per position based on their amino acid frequencies which most closely resemble the degree of enrichment or enrichment score found in the analysis of the DMS data. For example, the codon selection for the rational library design can be based on the below equation. The amino acid positions identified in DMS analysis that have a positive enrichment score (e.g., ER >1, or log[ER]>0) were normalized according to their enrichment ratios and were converted to theoretical frequencies. Degenerate codon schemes were then selected which most closely reflect these frequencies as calculated by the mean squared error between the degenerate codon and the target frequencies.

$$Optimal Codon = {arg}_{x} min ( \frac{1}{n} {\sum }_{i = 1}^{n} {{w}_{n} ( {Y}_{n , deg} - {Y}_{n , target} )}^{2} ) \qquad ( 2 )$$

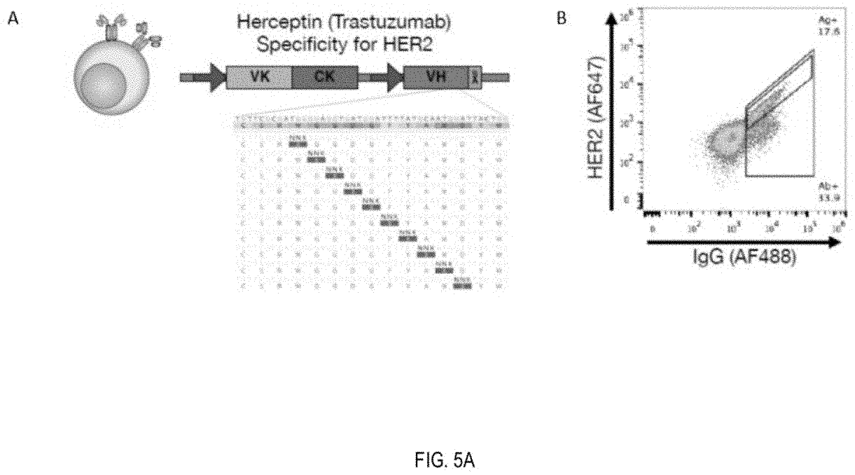

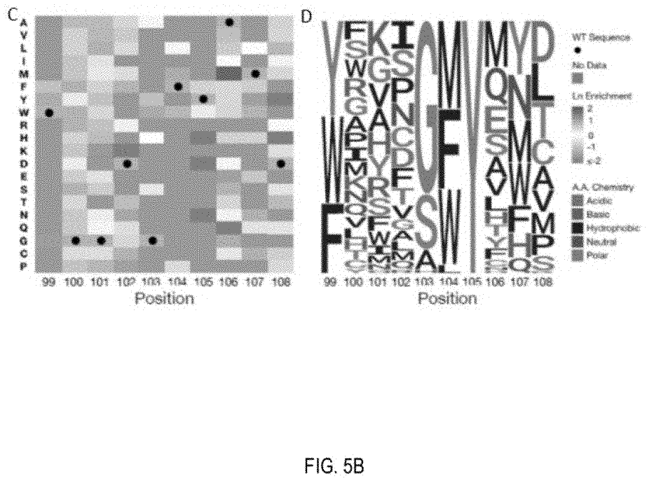

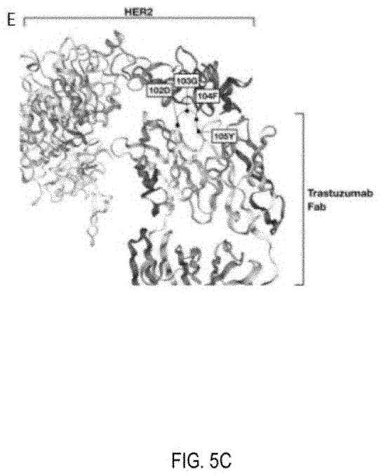

For example, the heatmap and sequence logo plots indicate that position 103 (FIG. 5) is highly acceptable of glycine (G) and serine (S) residues, and to a lesser extent alanine (A). The enrichment scores for these residues correspond to normalized frequencies of approximately 66% G, 25% S, and 9% A. These frequencies are then input values to the above optimal codon equation (e.g., Equation 2) and compared against all 3,375 possible degenerate codon schemes. In this example, the degenerate codon scheme ‘RGY’ was selected as it represents the degenerate codon scheme with the closest frequencies (50% G, 50% S) to the target frequencies defined by the normalized enrichment scores. Combining degenerate codons across multiple positions produces massive theoretical protein spaces. As an example, by taking the product of all potential amino acids per position across all positions, the combinatorial library generated for the trastuzumab antibody described in the Examples provided herein possessed a theoretical protein sequence space of 6.67×10⁸, which is far higher than the single-site DMS library diversity of 200. The combinatorial mutagenesis libraries containing CDRH3 variants can then be physically generated, e.g., in hybridoma cells through HDM. Antigen binding cells can then be isolated by one or more rounds of enrichment by FACS and the binding or non-binding populations subjected to deep sequencing. Sequencing data representing the binding or non-binding populations from this second step can then be employed as the training set for the machine learning model.

FIG. 4B illustrates a process flow 450 for selecting candidate variants. The process flow 450 can include training the models described herein with the trained data generated during the process flow 400. Once the training data is generated and the classification engine 108 is trained, the full sequence space of mutations can be generated in silico. The full sequence space can include each possible mutation. The number of variants in the full sequence space can be orders of magnitude larger than the number of variants on which the classification engine 108 was trained. The classification engine 108 can process the variants of the full sequence space to classify the variants as antigen-binder classified variants or non-antigen-binder classified variants. The process flow 450 can include the candidate selection engine 110 filtering the antigen-binder classified variants with multi-parameter optimization to select one or more candidate variants. The candidate selection engine 110 can filter antigen-binder classified variants by determining whether the antigen-binder classified variants satisfy a filtering policy. The filtering policy can include parameter requirements such as model consensus (e.g., did each of the LSTM neural network and convolutional neural network classify the variant as an antigen-binder classified variant), viscosity values, solubility values, stability values, pharmacokinetic values, and immunogenicity values.

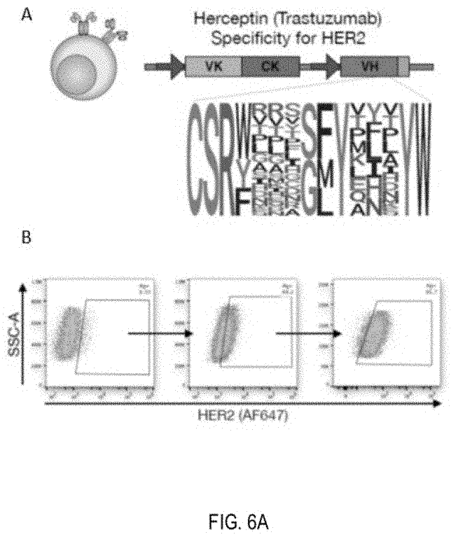

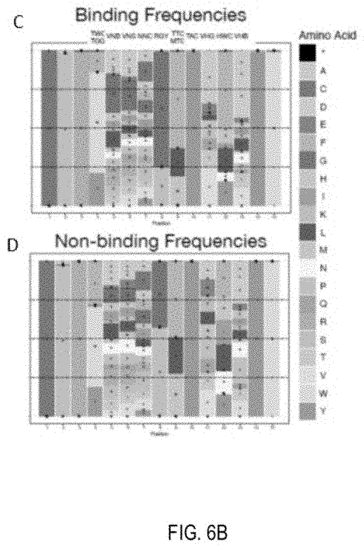

FIGS. 5 and 6 illustrate exemplary data for the process flow 400 and 450 as applied to the CDRH3 of exemplary antibody Trastuzumab, which are described in further detail in the Example below.

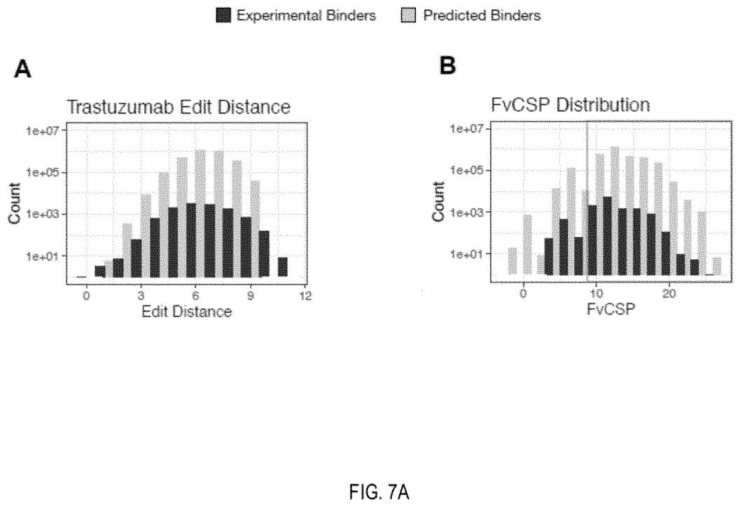

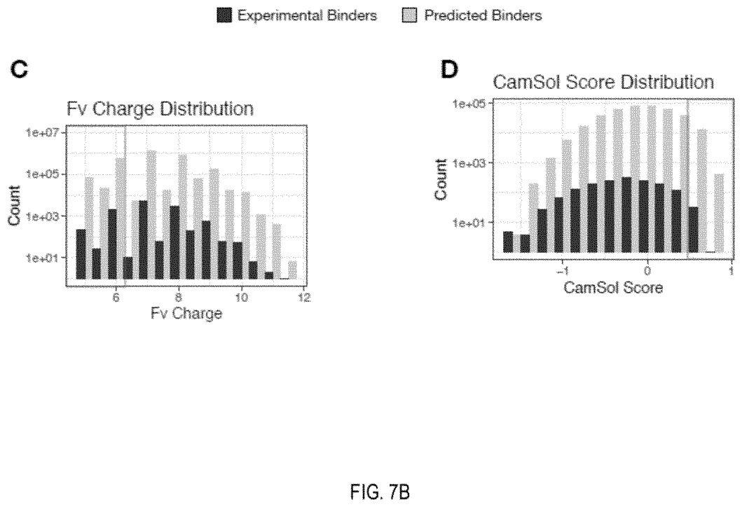

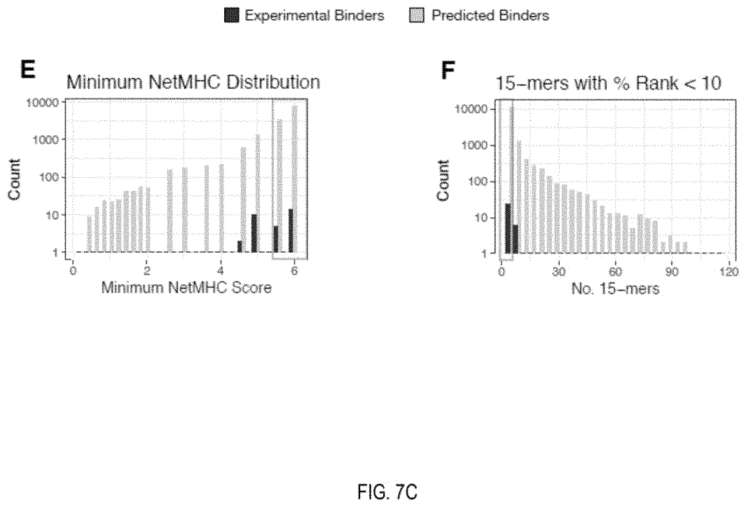

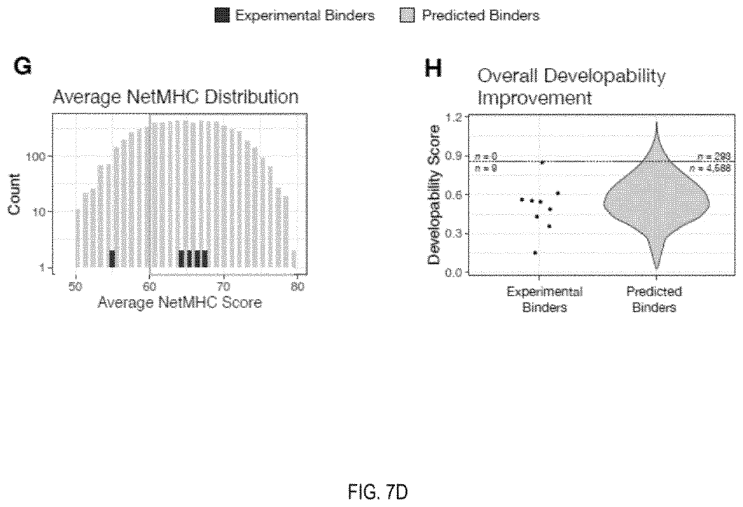

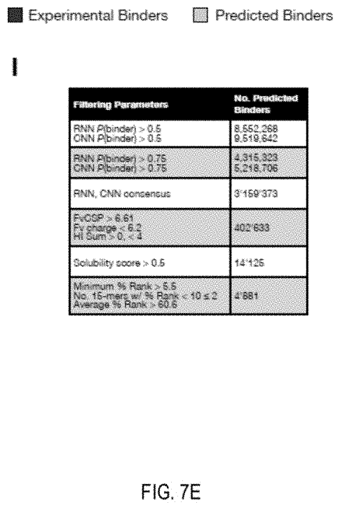

FIG. 7 illustrates a filtering policy 700 and a plurality of plots of parameters. As described above, for each of the antigen-binder classified variants, the candidate selection engine 110 can calculate parameter values. The system 100 can calculate, for example, a Levenshtein distance value, charge value, hydrophobicity index value, CamSol score, minimum affinity rank, and average affinity ranking for each antigen-binder classified variant. The system 100 can also identify within each of the antigen-binder classified variants sequence motifs associated with manufacturing liabilities, such as n-glycosylation sites, deamidation sites, isomerization sites, methionine oxidation, tryptophan oxidation and paired or unpaired cysteine residues.

The filtering policy 700 can include a plurality of parameter requirements. The candidate selection engine 110 can apply the parameter requirements in parallel. For example, the candidate selection engine 110 can calculate each of the parameter values for each of the antigen-binder classified variants and determine whether the antigen-binder classified variants satisfy the parameter requirements of the filtering policy 700. The candidate selection engine 110 can apply the parameter requirements in series. For example, the candidate selection engine 110 can sequentially calculate a parameter for the antigen-binder classified variants and determine whether the antigen-binder classified variant satisfies the parameters required for the given parameter. The system 100 may then only calculate the next parameter values for the antigen-binder classified variants that satisfied the first parameter requirement. When an antigen-binder classified variant does not satisfy a parameter requirement, the candidate selection engine 110 may not calculate the remaining parameter values for the antigen-binder classified variant. This can reduce the computational resources required to filter the antigen-binder classified variants as the parameter values are not calculated for the antigen-binder classified variants once they are removed by the filtering process. Thus, by determining to not calculate parameter values for antigen-binder classified variants that do not satisfy the parameter requirement, this technical solution can reduce computational resource consumption (e.g., processor utilization, memory utilization, or network bandwidth utilization), while identifying optimal variants.

Still referring to FIG. 7, the candidate selection engine 110 can first determine the antigen-binder classified variants output by the recurrent neural network (RNN) and the convolutional neural network (CNN). The candidate selection engine 110 can select only the variants that were classified by the respective neural network with a predetermined confidence. For example, as illustrated in FIG. 7, the candidate selection engine 110 can identify 4,315,323 antigen-binder classified variants identified by the recurrent neural network and 5,218,706 antigen-binder classified variants identified by the convolution neural network with a confidence or probability above 0.75. The next filter in the filtering policy 700 can include identifying antigen-binder classified variants identified by both the convolutional neural network and the recurrent neural network. The candidate selection engine 110 can identify 3,159,373 antigen-binder classified variants identified by both the convolutional neural network and the recurrent neural network with a probability greater than 0.75. The candidate selection engine 110 can then identify the antigen-binder classified variants with a charge symmetry parameter greater than 6.61, a net charge less than 0.2 and a hydrophobicity index less than 4, returning 402,633 antigen-binder classified variants. The candidate selection engine 110 can then identify antigen-binder classified variants with a solubility score greater than 0.5, returning 14,125 antigen-binder classified variants. The candidate selection engine 110 can then identify the antigen-binder classified variants with a NetMHCII minimum affinity rank greater than 5.5% and an average affinity rank greater than 60.6%, returning 4,881 antigen-binder classified variants. All remaining antigen-binder classified variants in this example contain values equal or greater than the parameters of the starting candidate sequence of trastuzumab. The candidate selection engine 110 can then identify the antigen-binder classified variants with the best overall developability across all parameters, returning the antigen-binder classified variants within the top percentage of the remaining candidate variants according to a predefined percentage. The system 100 can additionally identify the antigen-binder classified variants with a Levenshtein distance less than 5.

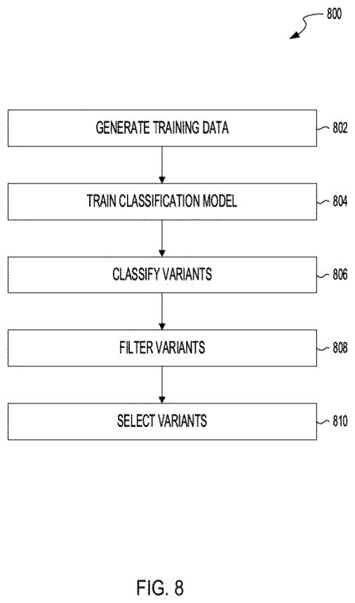

FIG. 8 illustrates a block diagram of an example method 800 to identify antibodies with an antigen affinity. The method 800 can include generating the training data (ACT 802). The method 800 can include training the classification model (ACT 804). The method 800 can include classifying variants (ACT 806). The method 800 can include filtering the variants (ACT 808). The method 800 can include selecting variants (ACT 810).

As set forth above, the method 800 can include generating training data (ACT 802). Also, with reference to FIG. 1, among others, the classification engine 108 can use the training data 118 for training to determine the classifier weights 112 for classifying unseen variants. The training data 118 can be generated using a two-step process that includes a single-site mutation process followed by a DMS-based combinatorial process.

The method 800 can include training the classification model (ACT 804). As described above, the classification engine 108 can include one or more classification models. For example, the classification engine 108 can include a recurrent neural network or a convolution neural network. The classification engine 108 can include a recurrent neural network, a convolution neural network, a standard artificial neural network (ANN), a support vector machine (SVM), a random forest ensemble (RF) or logistic regression (LR) model. The training data 118 can be labeled and passed to the neural networks as a one-hot encoded matrix. The classification engine 108 can use back-propagation and gradient descent to minimize the cost or error between the expected result and result determined by the classification engine 108. Once the classification engine 108 has trained its neural network, the classification engine 108 can save the weights and biases to the memory 106 as classifier weights 112.

The method 800 can include classifying variants (ACT 806). In some implementations, for a given antibody, the candidate identification system 102 can in silico generate the complete sequence space for the variants of the antibody. For example, the candidate identification system 102 can generate all possible sequence variations for a given antibody or portion thereof. The classification engine 108 can load the classifier weights 112. The classification engine 108 can pass each of the variants of the complete sequence space to the input layers of the convolutional neural network and recurrent neural network. For example, each variant, the classification engine 108 can determine a probability that the variant is an antigen-binder classified variant. The classification engine 108 can save the antigen-binder classified variants with a probability above a threshold as antigen-binder classified variants in the memory 106.

The method 800 can include filtering the antigen-binder classified variants (ACT 808). The candidate selection engine 110 can filter the antigen-binder classified variants to identify candidate variants. The candidate variants can be the antigen-binder classified variants that have the greatest probability of yielding viable antibodies. The candidate selection engine 110 can retrieve a filtering policy from the memory 106. The filtering policy can include a plurality of parameters that the antigen-binder classified variants must satisfy to be selected as a candidate variant. The candidate selection engine 110 can calculate the parameters for the antigen-binder classified variants and determine if each of the respective antigen-binder classified variants satisfy the parameter requirements of the filtering policy.

The method 800 can include selecting variants (ACT 810). The candidate variants (e.g., the antigen-binder classified variants that satisfy the parameters of the filtering policy) can be selected for further recombinant expression to test the variant produces an antibody with antigen-specific binding. In some implementations, a sub-portion of the candidate variants can be randomly selected for recombinant expression and testing.

While operations are depicted in the drawings in a particular order, such operations are not required to be performed in the particular order shown or in sequential order, and all illustrated operations are not required to be performed. Actions described herein can be performed in a different order.

The separation of various system components does not require separation in all implementations, and the described program components can be included in a single hardware or software product.

### I. Example

This Example describes an exemplary application of the systems and methods described herein to the CDRH3 of Trastuzumab (Herceptin) antibody and classify antibody binding to the corresponding target HER2 antigen.

### A. Results

1) Deep Mutational Scanning Determines Antigen-Specific Sequence Landscapes and Guides Rational Antibody Library Design

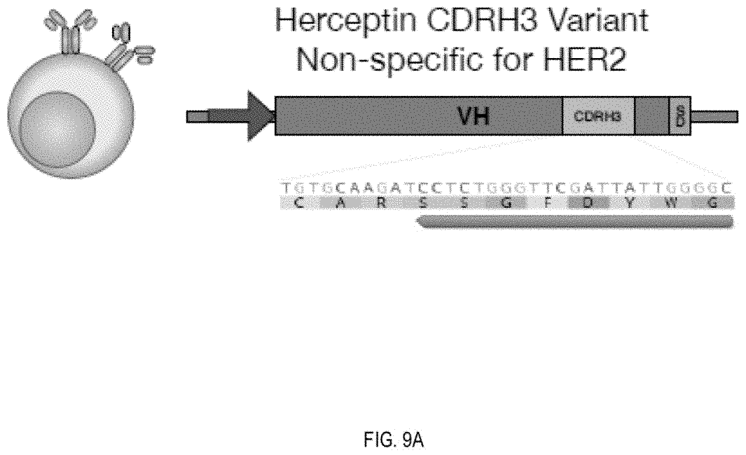

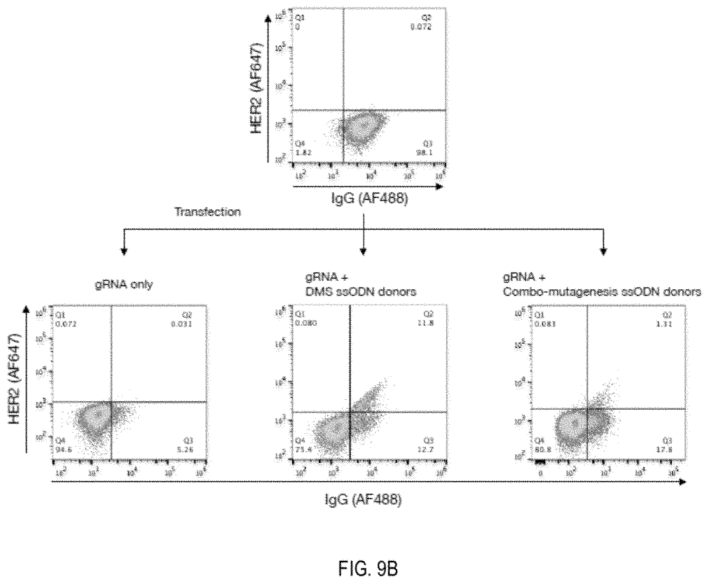

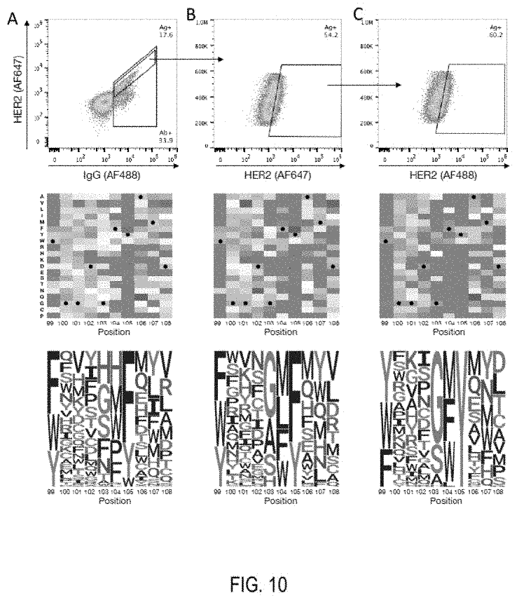

As the amino acid sequence of an antibody's CDRH3 is a key determinant of antigen specificity, deep mutational scanning (DMS) was performed on this region to resolve the specificity determining residues. To start, a hybridoma cell-line expressing a trastuzumab variant that could not bind HER2 antigen (mutated CDRH3 sequence) was used (FIG. 9). Libraries were generated by CRISPR-Cas9-mediated homology-directed mutagenesis (HDM) (Mason et al. (2018) Nucleic Acids Research 46 (14): 7436-49), which utilized gRNA for Cas9 targeting of CDRH3 and a pool of homology templates in the form of single-stranded oligonucleotides (ssODNs) containing NNK degenerate codons at single-sites tiled across CDRH3 (FIG. 5A). Libraries were then screened by fluorescence activated cell sorting (FACS), and populations expressing antibody and binding or not binding antigen were subjected to deep sequencing (Illumina MiSeq) (FIG. 10). Deep sequencing data was then used to calculate enrichment scores, of the 10 positions investigated, which revealed six positions that were sufficiently amenable to a wide-range of mutations with an additional three positions that were marginally accepting to defined mutations (FIGS. 5B and 5C). Although residues 103 102D, 103G, 104F, and 105Y appear to be the primary contacting amino acids of the CDRH3 loop with HER2 (PDB ID:1N8Z, Cho et al. (2003) Nature 421 (6924): 756-60, Rose et al. (2018) Bioinformatics 34 (21): 3755-58, 105Y is the only residue completely fixed (FIG. 5D).

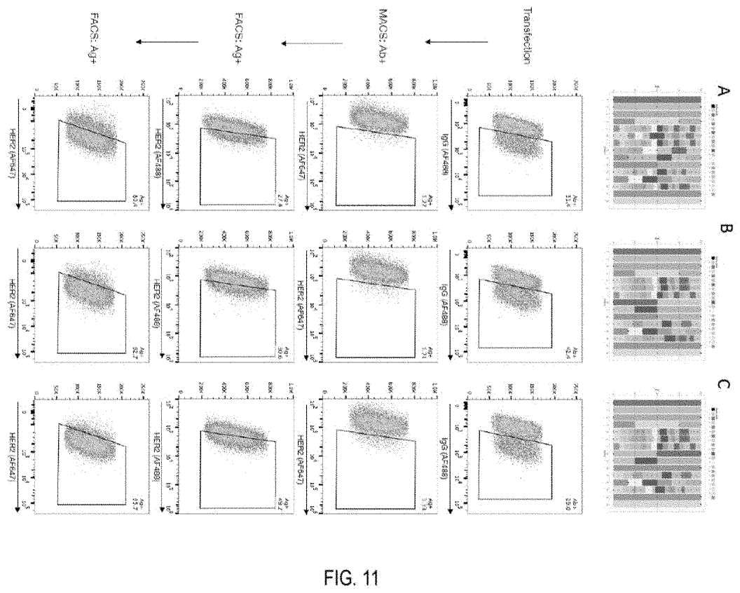

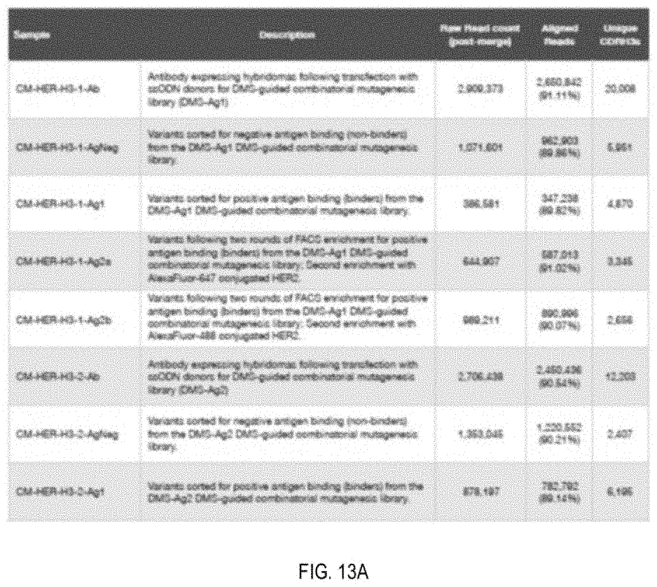

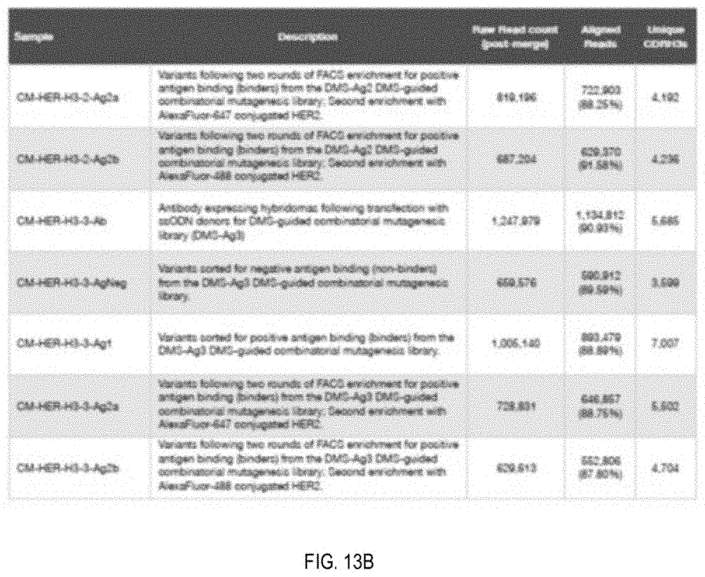

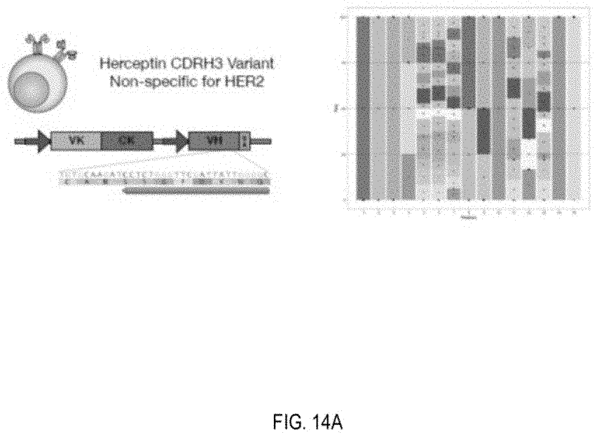

Heatmaps and their corresponding sequence logo plots generated by DMS were used to guide the rational design of a combinatorial mutagenesis library, which consisted of degenerate codons across all positions (except 105Y) (FIG. 11). Degenerate codons were selected per position based on their amino acid frequencies which most closely resembled the degree of enrichment found in the DMS data (FIG. 5C, Equation 2). This combinatorial library possesses a theoretical protein sequence space of 6.67×10⁸ which is far greater than the single-site DMS library diversity of 200. The theoretical diversity can be calculated by taking the product of all possible amino acids per position across all positions (e.g., all 20 amino acids present at all positions results in 20\hat{ }X, where X is the number of positions). In some implementations, DMS-guided combinatorial mutagenesis libraries can have a reduced subset of amino acids per position, resulting in a reduction of theoretical diversity. Libraries containing CDRH3 variants were again generated in hybridoma cells through HDM in the same non-binding trastuzumab clone described previously (FIG. 6A). Antigen binding cells were isolated by two rounds of enrichment by FACS and the binding/non-binding populations were subjected to deep sequencing. Sequencing data identified 11,300 and 27,539 unique binders and non-binders, respectively (NGS statistics, FIG. 13). These sequence variants represented only a miniscule 0.0058% of the theoretical protein sequence space of the combinatorial mutagenesis library. Amino acid usage per position was comparatively similar between binding and non-binding populations (FIG. 6B), thus making it difficult to develop any sort of heuristic rules or observable patterns to identify binding sequences.

2) Training Deep Neural Networks to Classify Antigen-Specificity Based on Antibody Sequence

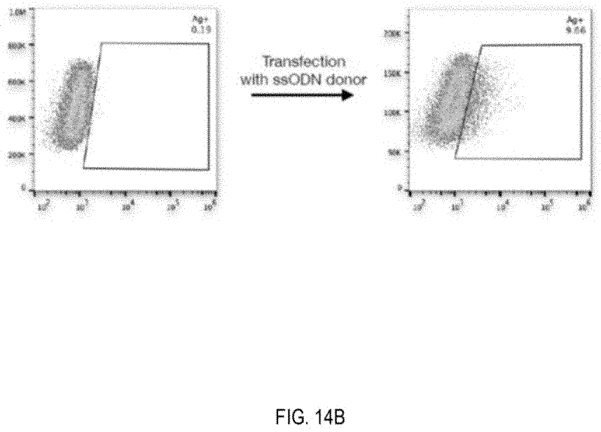

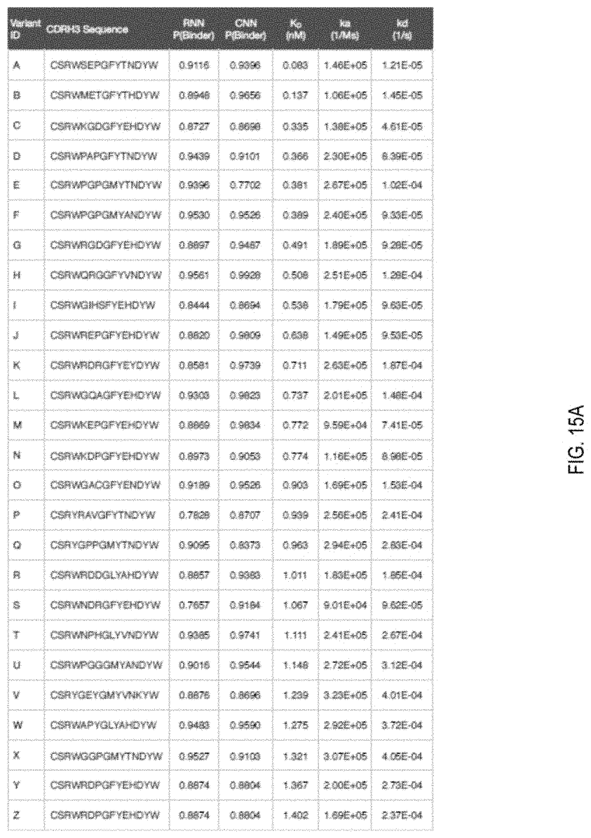

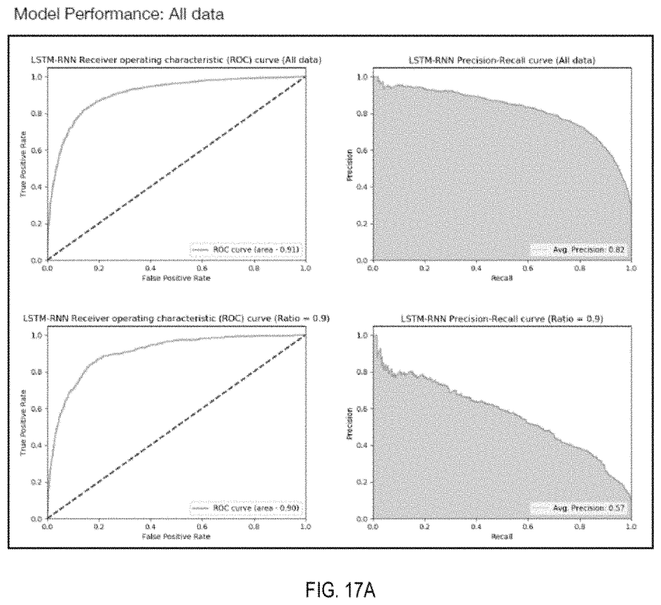

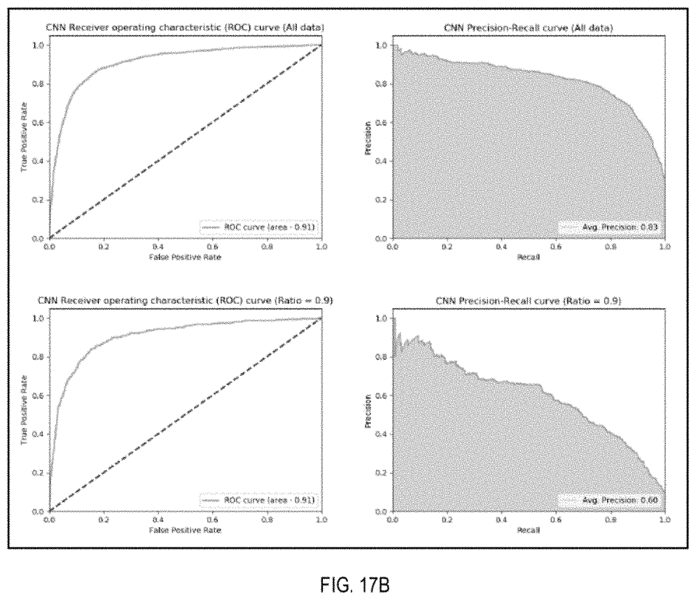

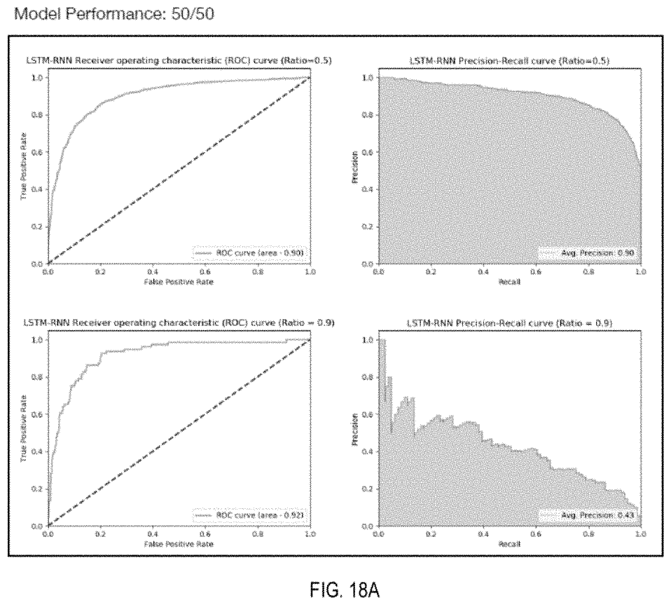

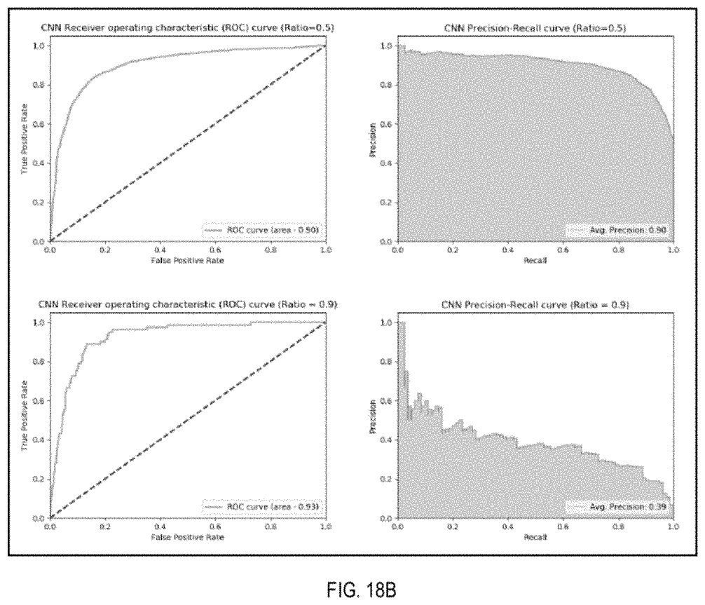

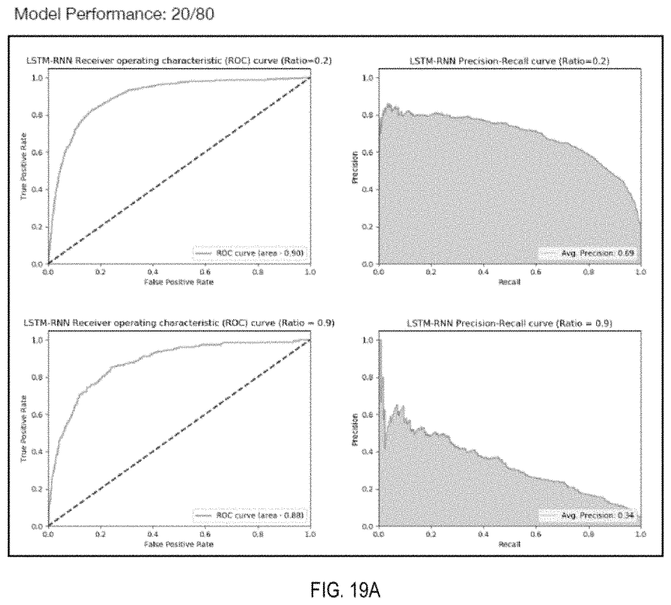

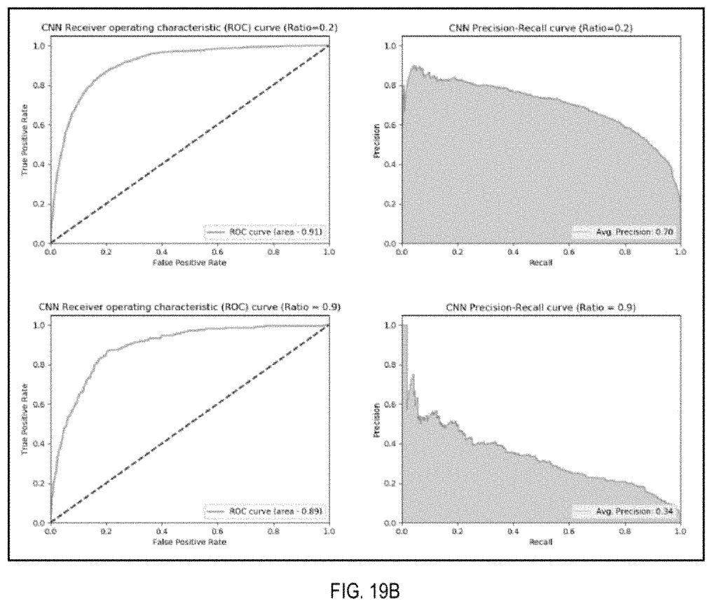

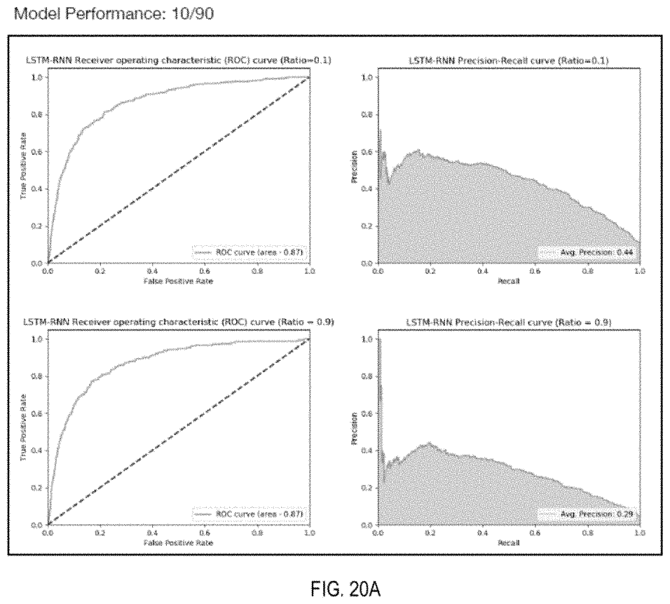

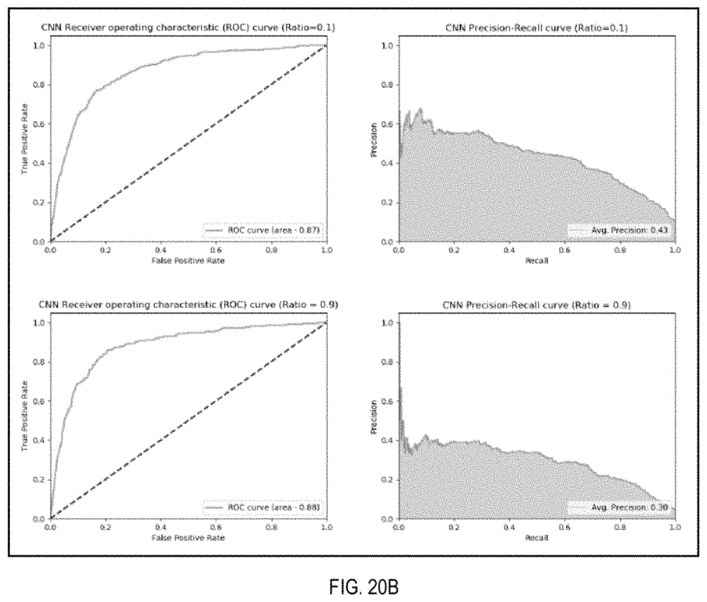

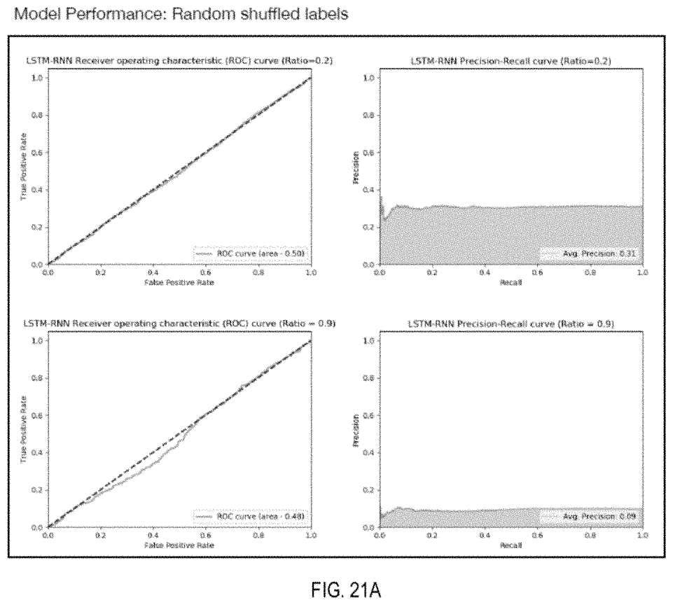

After having compiled deep sequencing data on binding and non-binding CDRH3 variants, deep learning models capable of predicting specificity towards the target antigen HER2 were developed and trained. Amino acid sequences were converted to an input matrix by one-hot encoding, an approach where each column represents a specific residue and each row corresponds to the position in the sequence, thus a 10 amino acid CDRH3 sequence as here results in a 10×20 matrix. Each row will contain a single ‘1’ in the column corresponding to the residue at that position, whereby all other columns/rows receive a ‘0’. LSTM-RNNs and CNN. LSTM-RNNs and CNNs both stem from standard neural networks, where information is passed along neurons that contain learnable weights and biases, however, there are fundamental differences in how the information is processed. LSTM-RNN layers contain loops, enabling information to be retained from one step to the next, allowing models to efficiently correlate a sequential order with a given output; CNNs, on the other hand, apply learnable filters to the input data, allowing it to efficiently recognize spatial dependencies associated with a given output. Model architecture and hyperparameters were selected by performing a grid search across various parameters (LSTM-RNN: nodes per layer, batch size, number epochs, and optimizing function; CNN: number of filters, kernel size, dropout rate, dense layer nodes) using a k-fold cross-validation of the data set (FIG. 7). All models were built to assess their accuracy and precision of classifying binders and non-binders from the available sequencing data. 70% of the original data set was used to train the models and the remaining 30% was split into two test data sets used for model evaluation: one test data set contained the same class split of sequences used to train the model and the other contained a class split of approximately 10/90 binders/non-binders to resemble physiological frequencies (FIGS. 6A and 14). Performance of the LSTM-RNN and CNN were assessed by constructing receiver operating characteristic (ROC) curves and precision-recall (PR) curves derived from predictions on the unseen testing data sets. Based on conventional approaches to training classification models, the data set was adjusted to allow for a 50/50 split of binders and non-binders during training. Under these training conditions, LSTM-RNN and CNN were able to accurately classify unseen test data (ROC curve AUC: 0.9±0.0, average precision: 0.9±0.0, FIG. 17).

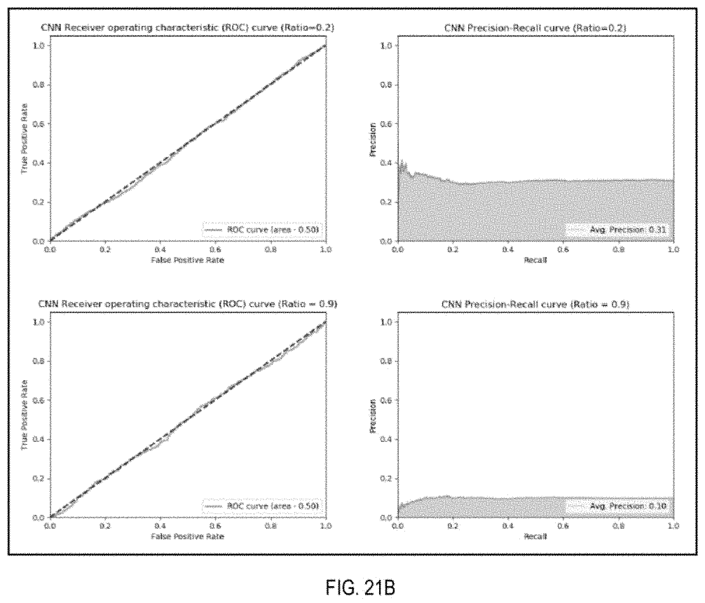

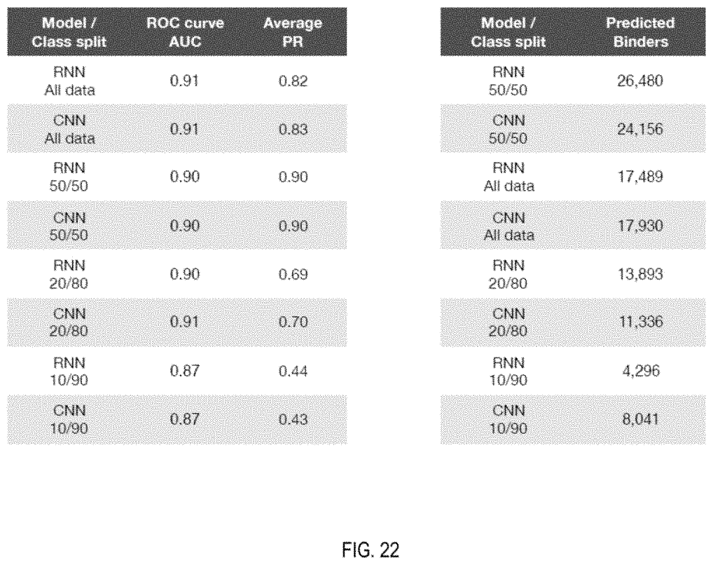

Next, the trained LSTM-RNN and CNN models were used to classify a random sample of 1×10⁵ sequences from the potential combinatorial diversity space were used. However, an unexpectedly high occurrence of positive classifications (25,318±1,643 sequences or 25.3±1.6%, FIG. 21) was observed. In view of the knowledge that the physiological frequency of binders should be approximately 10-15%, the classification split of the training data with the hypothesis that models were being subject to some unknown classification bias was adjusted. Additional models were then trained on classification splits of both 20/80, and 10/90 binders/non-binders, as well as a classification split with all available data (approximately 30/70 binders/non-binders). Unbalancing the sequence classification led to a significant reduction in the percentage of sequences classified as binders, but also led to a reduction in the model performance on the unseen test data (FIG. 21). Through this analysis, it was concluded that the optimal data set for training the models was the set inclusive of all known CDRH3 sequences for the following reasons: 1) the percentage of sequences predicted as binders reflects this physiological frequency, 2) this data set maximizes the information the model sees, and 3) model performance on the test data. Final model architecture, parameters, and evaluation are shown in FIG. 2.

3) Multi-Parameter Optimization for Developability by in Silico Screening of Antibody Sequence Space

Next, the full 3.1×10⁶ deep learning predicted antigen-specific sequences were characterized on a number of parameters to identify highly developable candidates compared to the original trastuzumab sequence. As a preliminary metric, their sequence similarity to the original trastuzumab sequence was investigated by calculating the LD. The majority of sequences showed an edit distance of LD>4 (FIG. 7A). The first step in filtering was to calculate the net charge and hydrophobicity index in order to estimate the molecule's viscosity and clearance. According to Sharma et al., viscosity decreases with increasing variable fragment (Fv) net charge and increasing Fv charge symmetry parameter (FvCSP); however, the optimal Fv net charge in terms of drug clearance is between 0 and 6.2 with a CDRL1+CDRL3+CDRH3 hydrophobicity index sum (HI sum)<4. Based on the wide range of values for these parameters in the 3.1×10⁶ predicted variants (FIG. 7B,C), we filtered any sequences out that had a FvCSP <6.61 (trastuzumab FvCSP) or if they contained a Fv net charge >6.2, and an HI sum >4, <0. This filtering criteria greatly reduced the sequence space down to 4.02×10⁵ variants. We next padded the CDRH3 sequences with 10 amino acids on the 5′ and 3′ ends and then ran these sequences through CamSol, a protein solubility predictor developed by Sormanni et al., which estimates and ranks sequence variants based on their theoretical solubility. The remaining variants produced a wide-range of protein solubility scores (FIG. 7D) and sequences with a score <0.5 (trastuzumab score) were filtered out, leaving 14,125 candidates for further analysis. As a last step in the in silico screening process, we aimed at reducing immunogenicity by predicting the peptide binding affinity of the variant sequences to MHC Class II molecules by utilizing NetMHCIIpan, a model previously developed by Jensen et al. One output from the model is a given peptide's % Rank of predicted affinity compared to a set of 200,000 random natural peptides. Typically, molecules with a % Rank <2 are considered strong binders and those with a % Rank <10 are considered weak binders to the MHC Class II molecules scanned. All possible 15-mers from the padded CDRH3 sequences were run through NetMHCIIpan. After predicting the affinities for a set of 26 HLA alleles determined to cover over 98% of the global population32, sequences were filtered out if any of the 15-mers contained a % Rank <5.5 (trastuzumab minimum % Rank) (FIG. 7E). The number of 15-mers with a % Rank less than 10 and the average % Rank across all 15-mers for the remaining sequences were also calculated. Sequences with more than two 15-mers with a % Rank <10 (FIG. 7F) and those with an average % Rank <60.56 (trastuzumab average % Rank) were also filtered out (FIG. 7G). All remaining 4,881 variants contain values equal to or greater than the parameters of the original trastuzumab sequence. When applying this same filtering scheme on the 11,300 experimentally determined binding sequences (obtained from training/test data), only 9 variants remained. Lastly, to determine the best developable sequences, we calculated an overall developability improvement score based on the mean of normalized values for each relevant parameter (see Materials and Methods), where trastuzumab would have a developability improvement score equal to 0. Of the remaining 4,881 predicted binding sequences, 293 variants were identified to have a higher developability score compared to the maximum developability score of the 9 experimentally determined binding sequences (FIG. 7H). The filtering parameters and number of remaining variants at each step for the in silico library are provided in FIG. 7I.

4) Selected Antibody Sequences are Recombinantly Expressed and Antigen-Specific

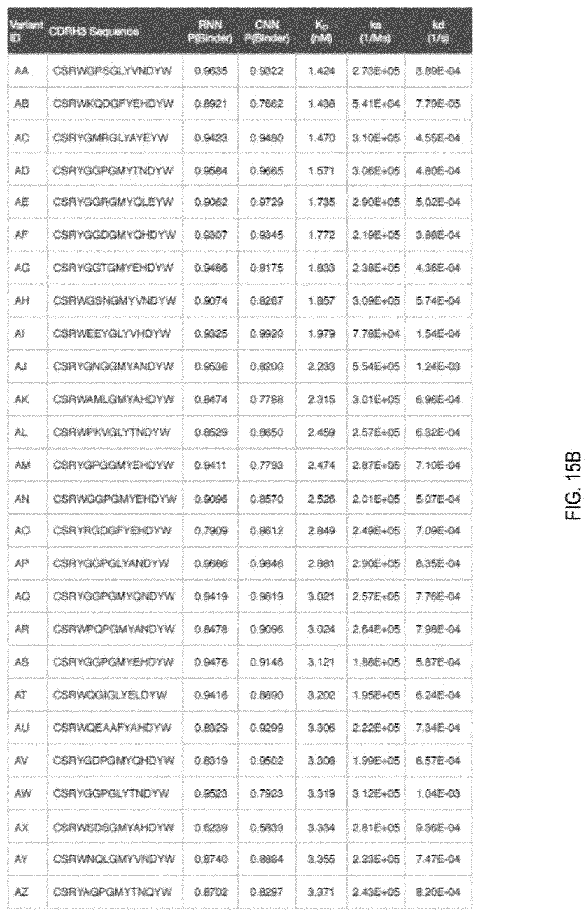

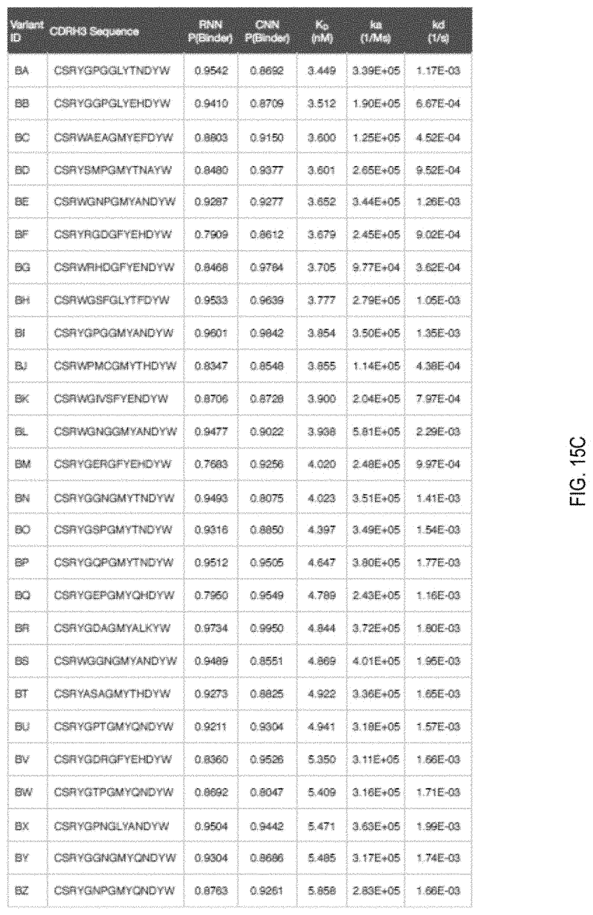

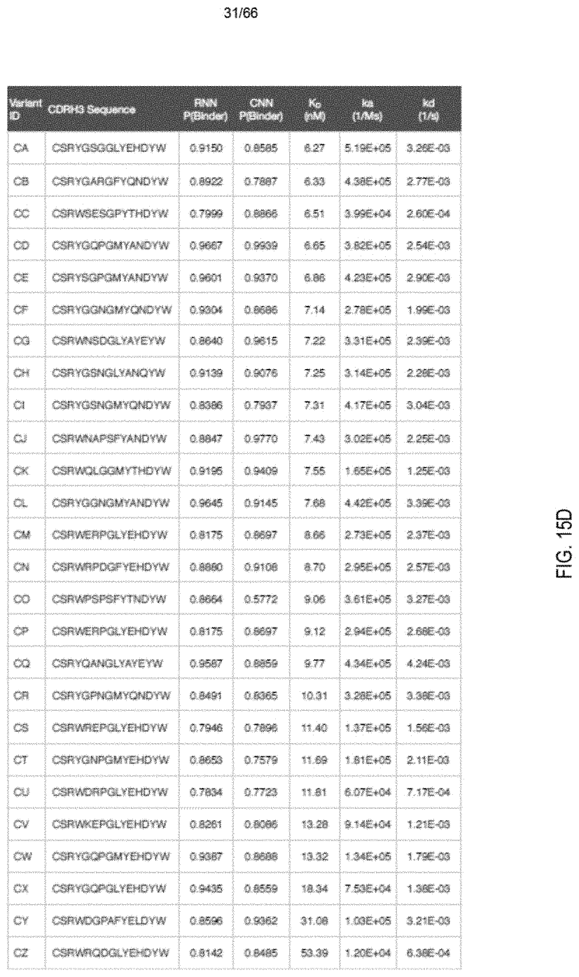

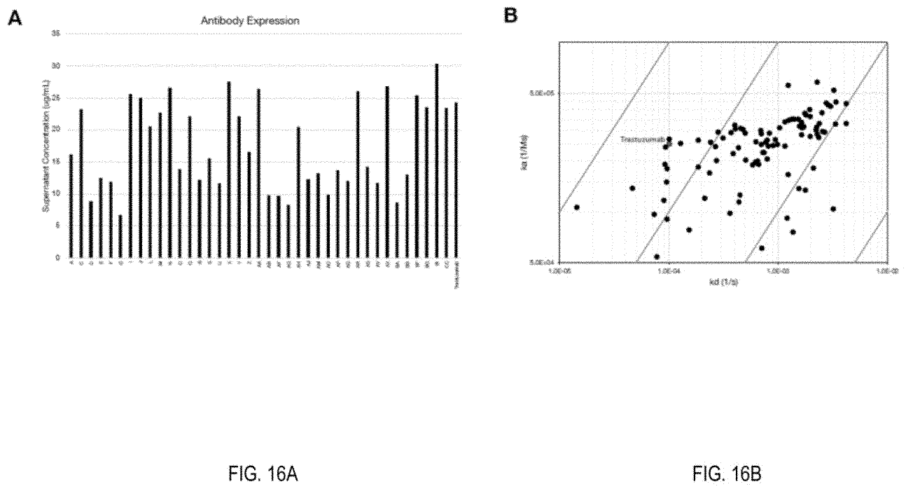

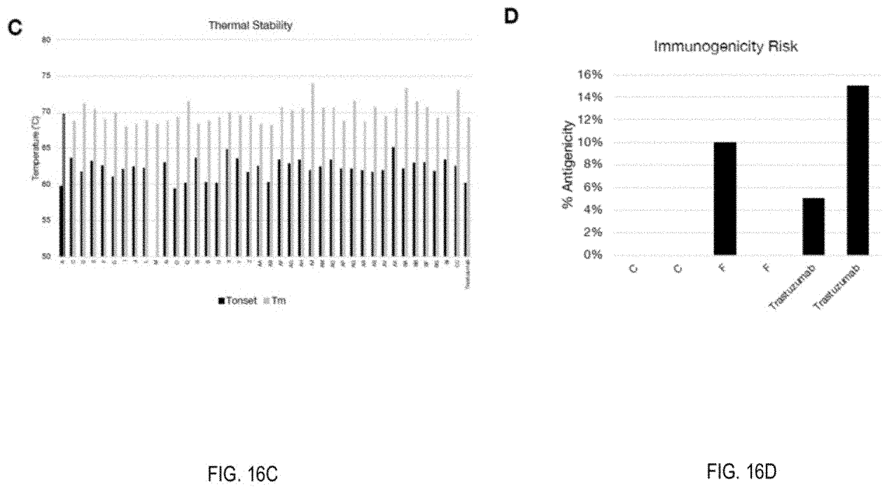

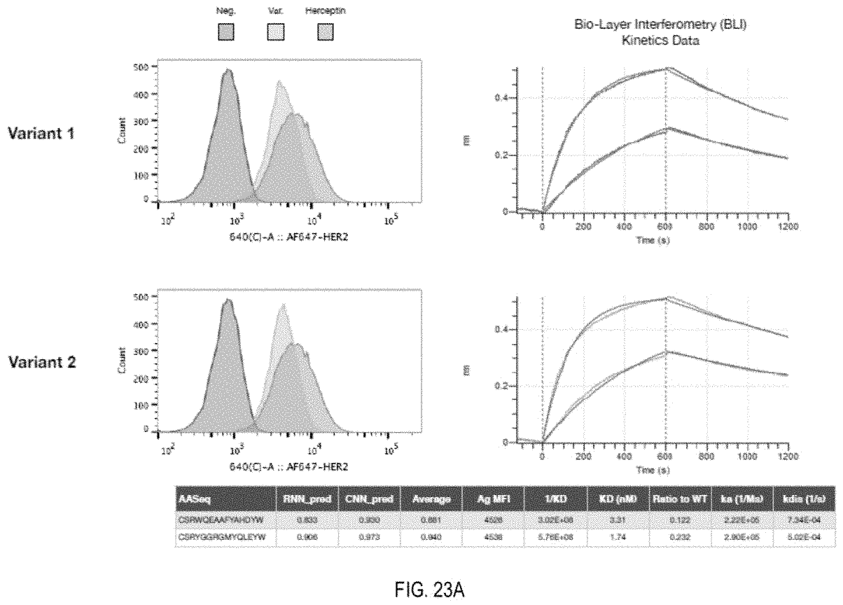

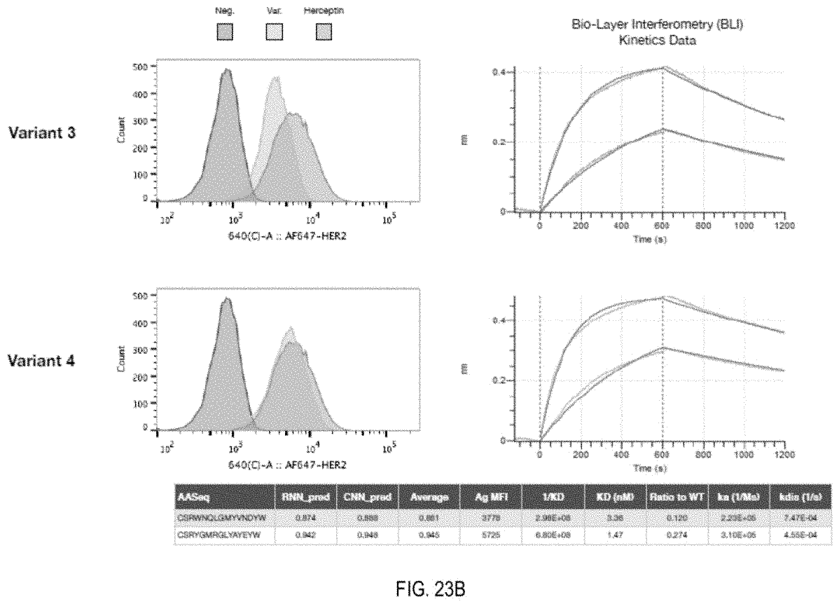

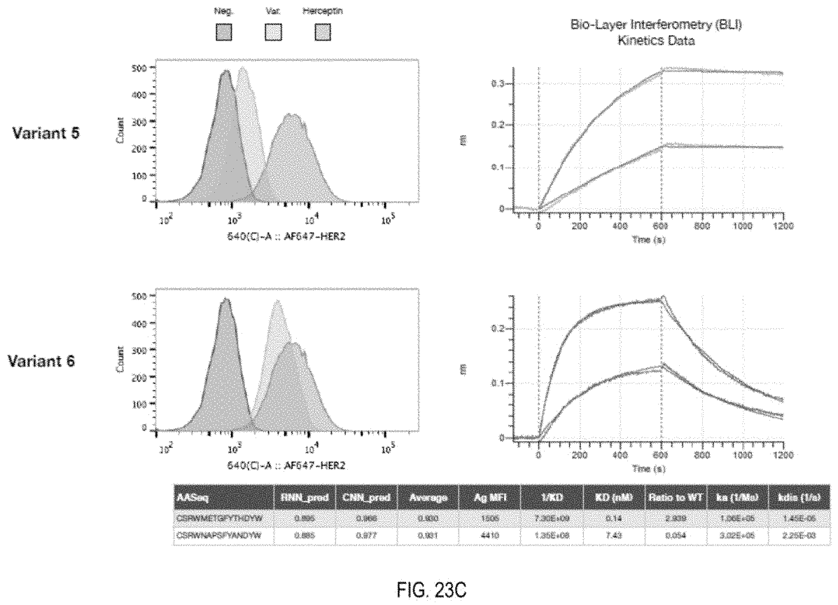

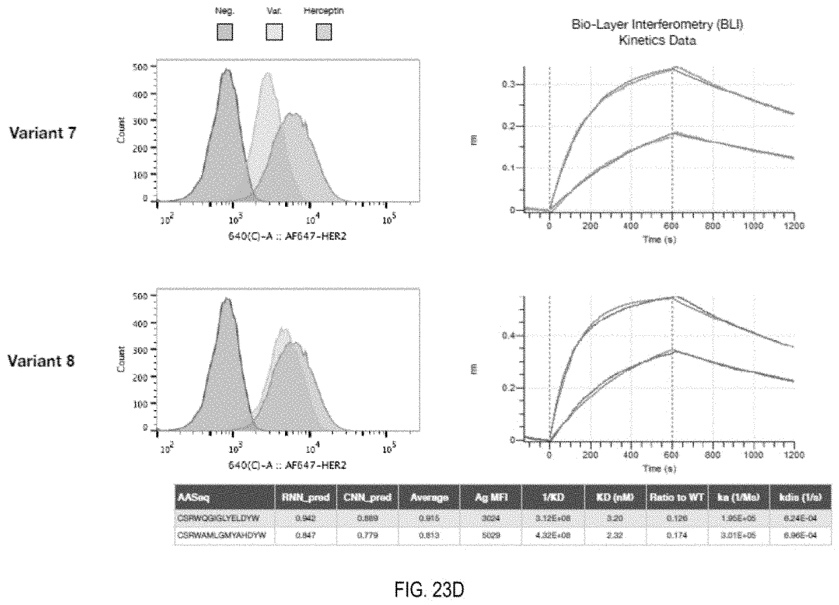

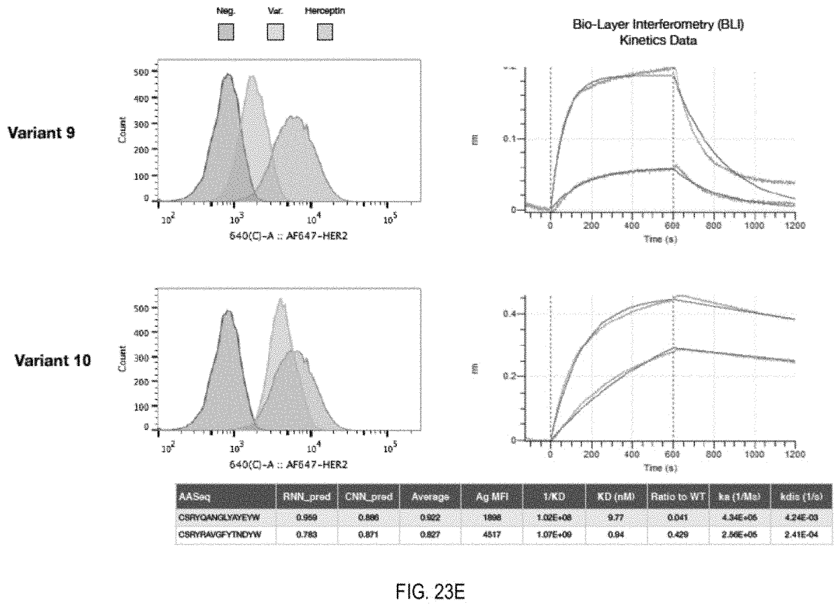

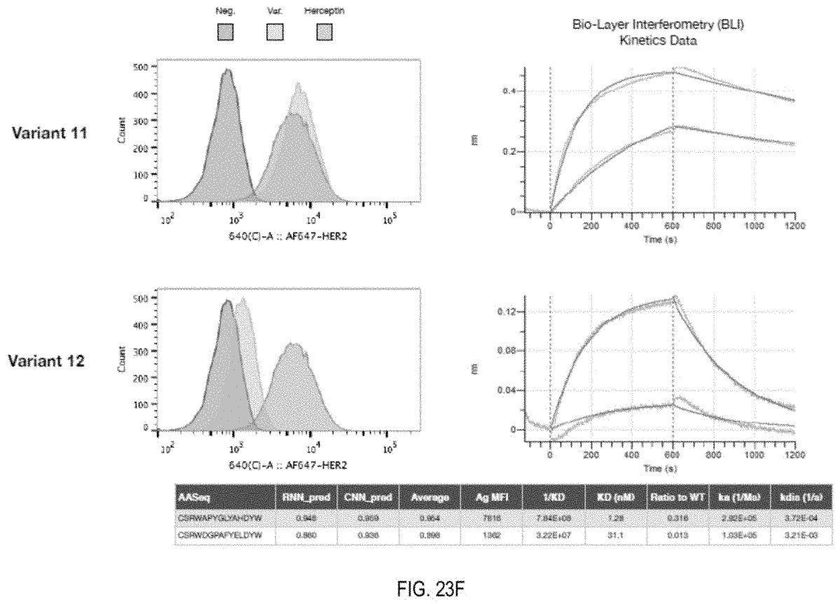

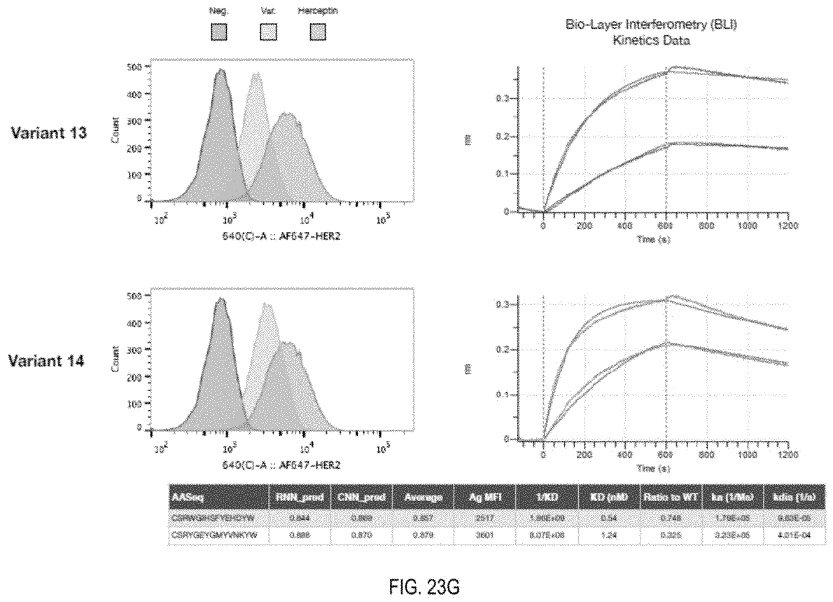

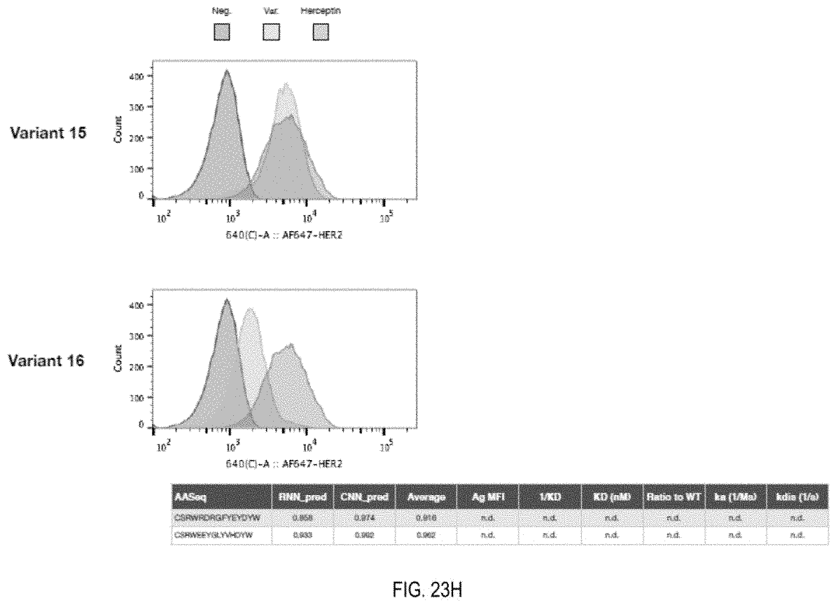

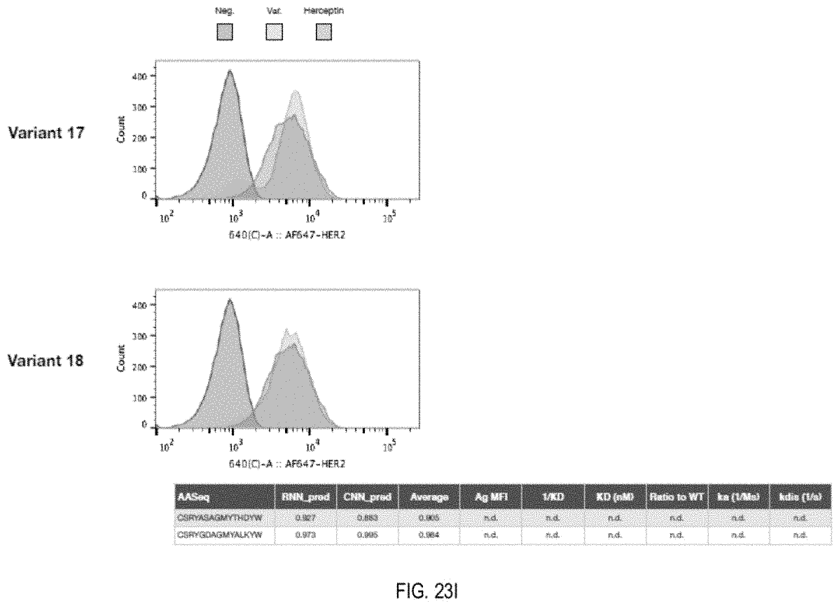

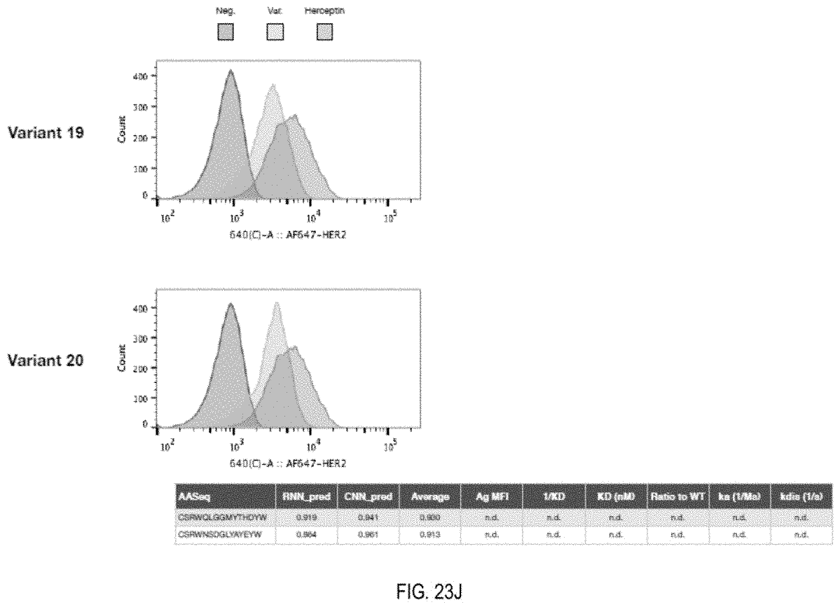

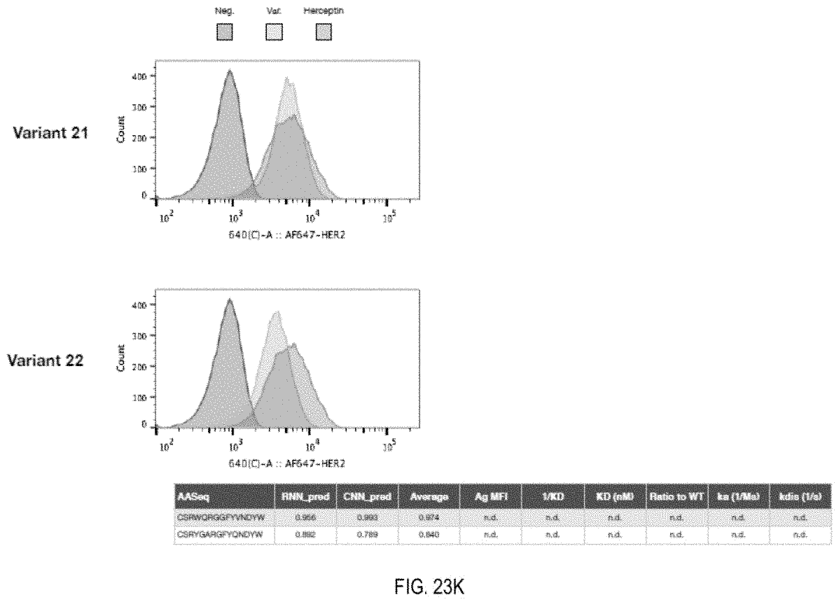

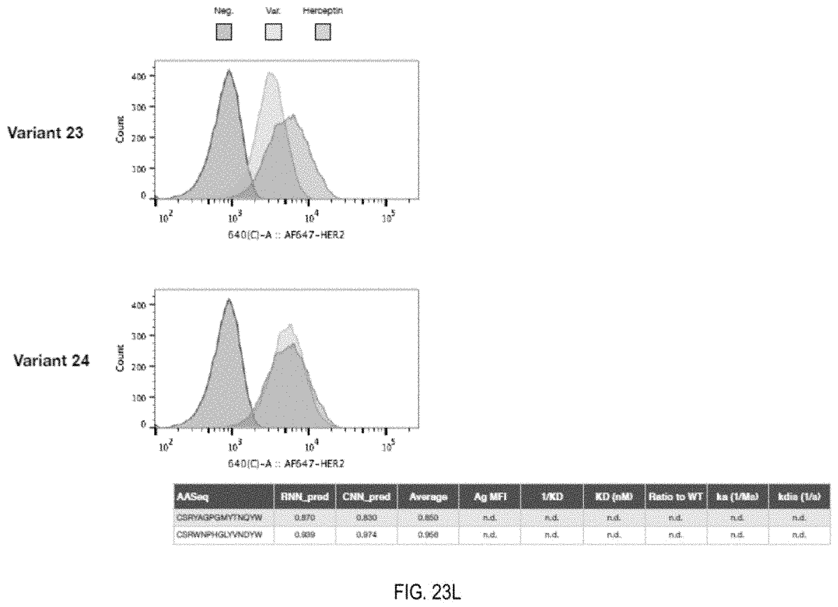

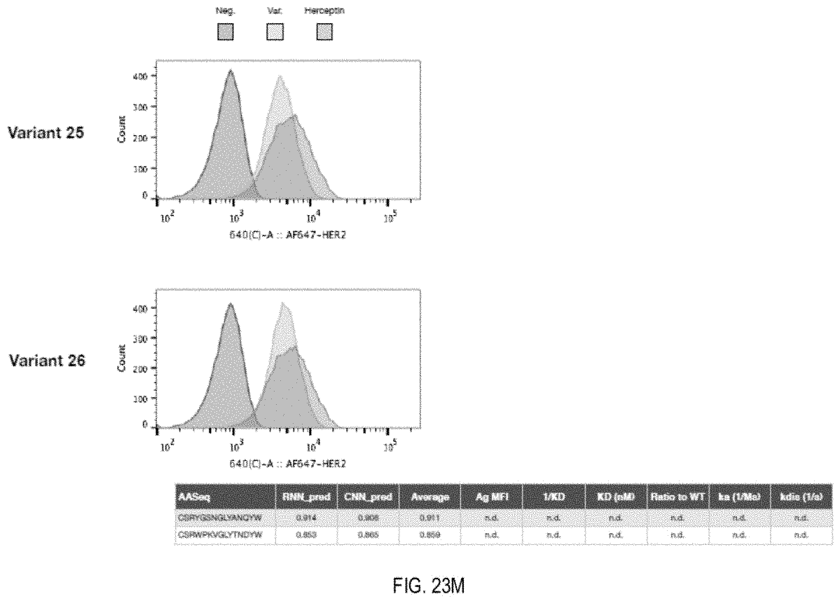

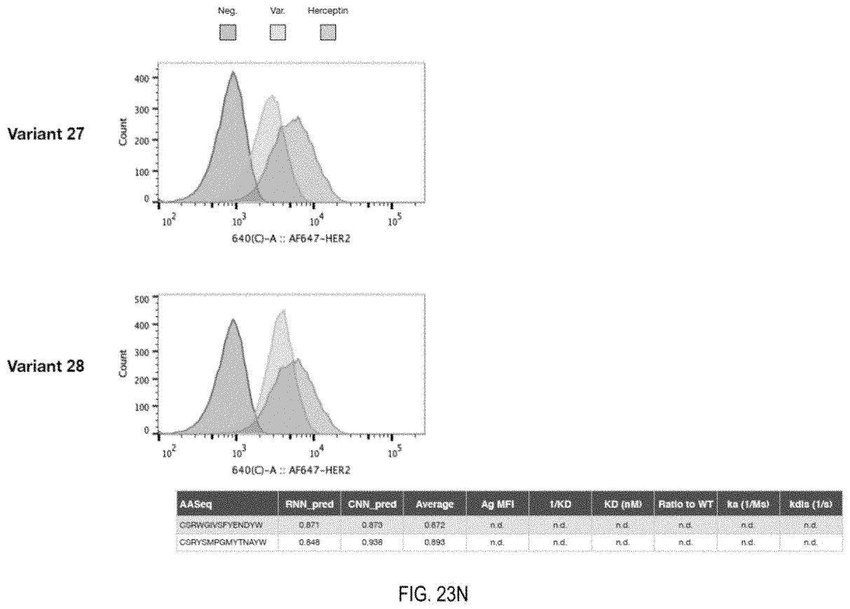

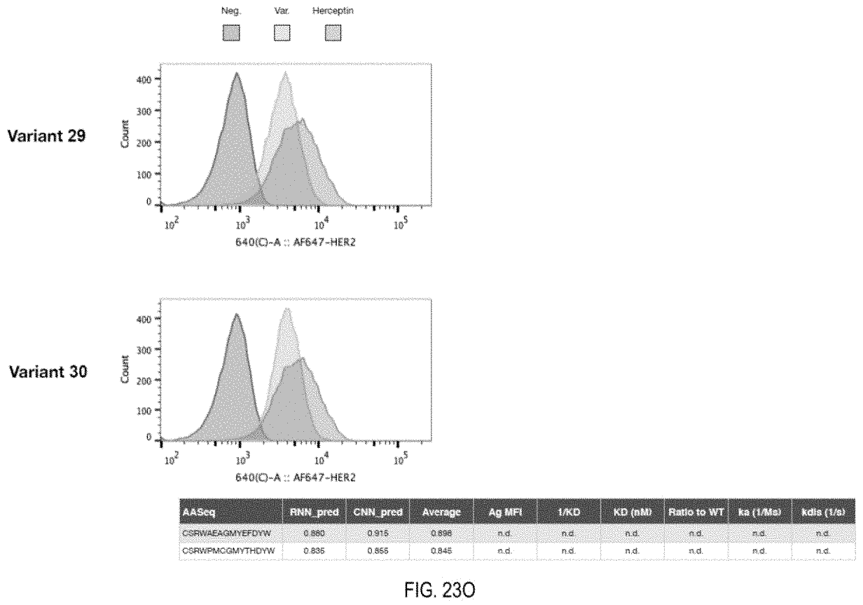

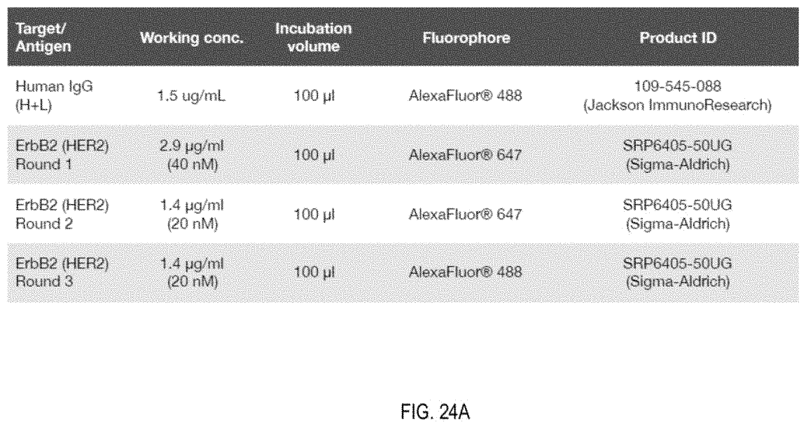

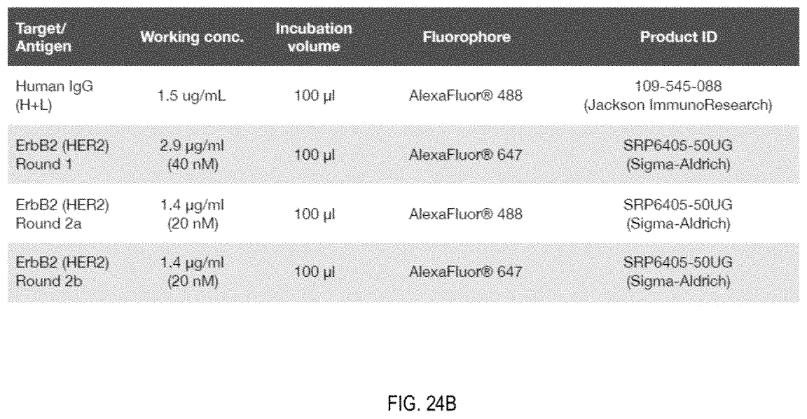

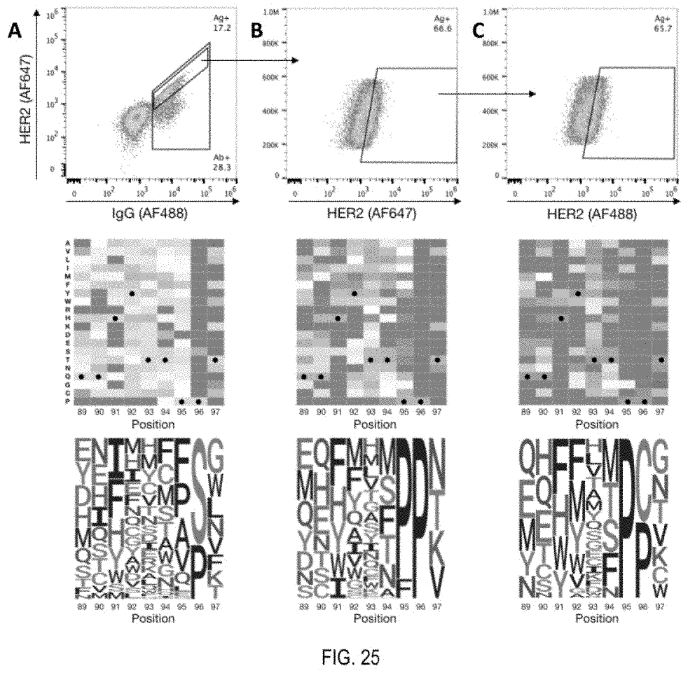

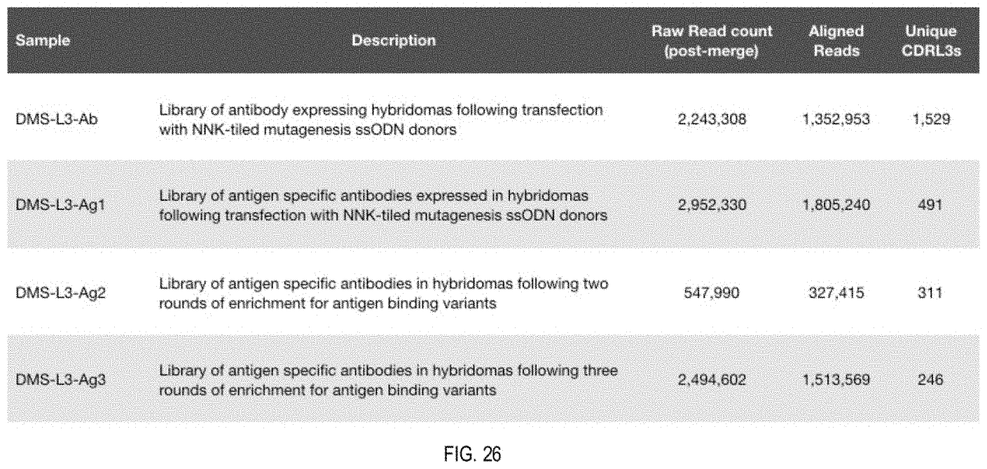

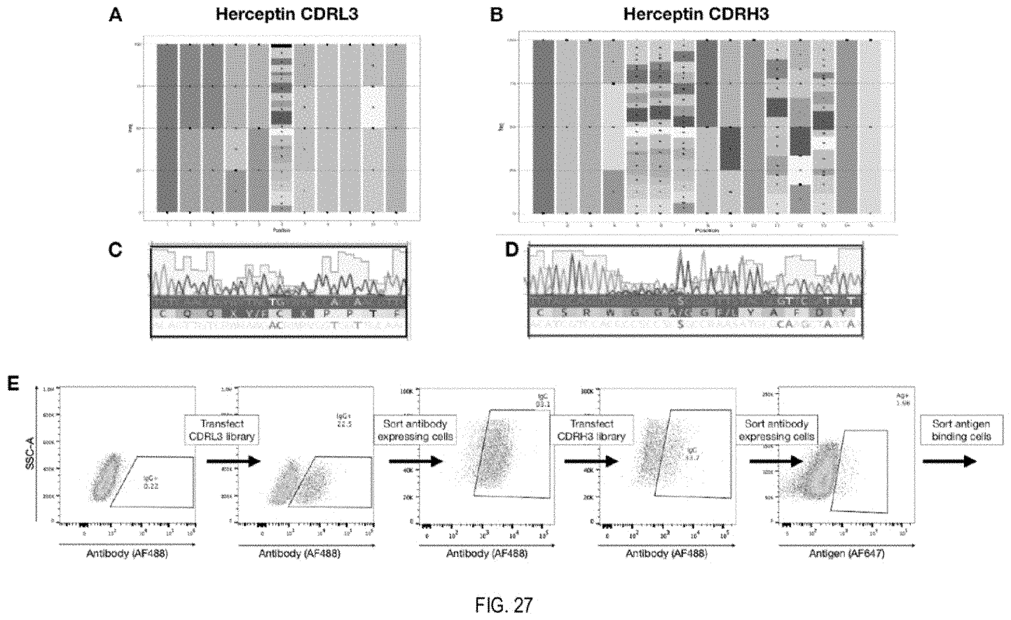

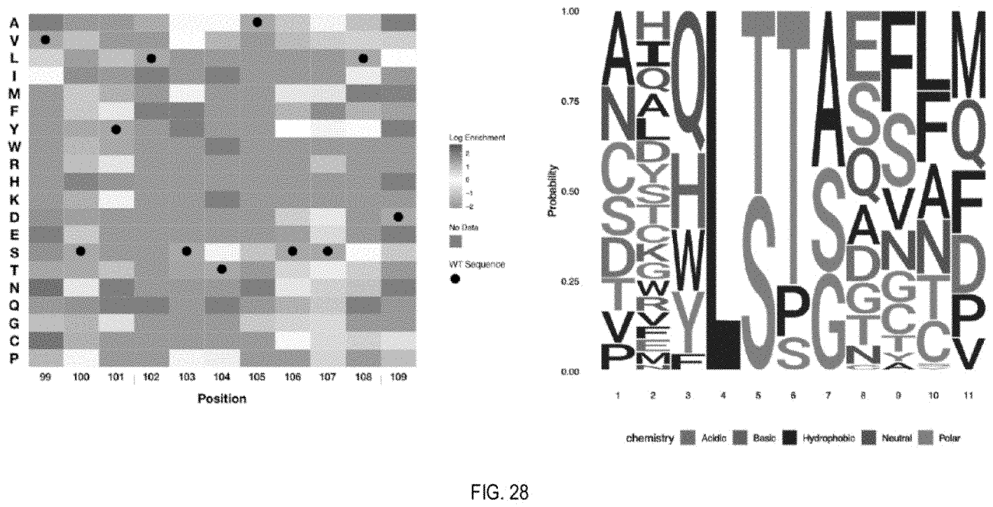

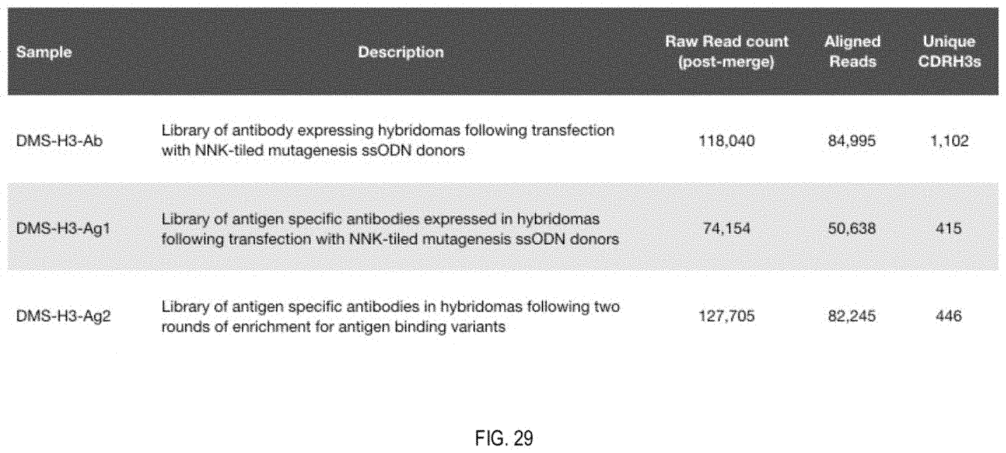

To validate the precision of the fully trained LSTM-RNN and CNN models, a subset of 30 CDRH3 sequences predicted to be antigen-specific and optimized across the multiple developability parameters was randomly selected. To further demonstrate the capacity of deep learning to identify novel sequence variants, the criteria that the selected variants have a minimum Levenshtein edit distance of 5 from the original CDRH3 sequence of trastuzumab was also added. CRISPR-Cas9-mediated HDR was used to generate mammalian display cell lines expressing different sequence variants. Flow cytometry was performed and revealed 29 of the 30 variants (96.67%) were antigen-specific (FIG. 23 A-23 O) Further analysis was performed on 104 of the antigen-binding variants to more precisely quantify the binding kinetics via biolayer interferometry (FortéBio Octet) (FIG. 15, FIG. 16B, FIGS. 23A-G). The original trastuzumab sequence was measured to have an affinity towards HER2 of 4.0×10⁻¹⁰ M (equilibrium dissociation constant, Kd); and although the majority of variants tested had a slight decrease in affinity, 75% (78/104) were still in the single-digit nanomolar range, 16% (17/104) remained sub-nanomolar, and six variants (5%) showed an increase in affinity compared to trastuzumab (Kd=1.4×10⁻¹⁰ M).

Developability parameters for the selected variants were also experimentally validated. In particular, expression levels of the selected variants were compared to those of trastuzumab (FIG. 16A). Further, thermal stability of the selected variants were compared to those of trastuzumab. (FIG. 16C). Immunogenicity risk was also compared to trastuzumab, where each tested variant (variants C and F) and trastuzumab were each tested twice (FIG. 16D).

### B. Discussion

Addressing the limitation of antibody optimization in mammalian cells, an approach based on deep learning has been developed that enables identification of antigen specific sequences with high precision. Using the clinically approved antibody trastuzumab, single-site DMS was performed followed by combinatorial mutagenesis to determine the antigen-binding landscape of CDRH3. This DMS-based mutagenesis strategy was important for attaining high quality training data that is enriched with antigen-binding variants, in this case nearly 10% of the generated library (FIG. 14). In contrast, if a completely randomized combinatorial mutagenesis strategy was employed (i.e., NNK degenerate codons), it would be unlikely to produce any significant fraction of antigen-binding variants.

A remarkable finding in this study was that experimental screening of a library of only 5×10⁴ variants, which reflected a tiny fraction (0.0005%) of the total sequence diversity of the DMS-based combinatorial mutagenesis library (6.67×10⁸), was capable of training accurate neural networks. This suggests that physical library size limitations of mammalian expression systems (or other expression systems such as phage display and yeast display) and deep sequencing read depth will not serve as a limitation in deep learning-guided protein engineering. Another important result was that deep sequencing of antigen-binding and non-binding populations showed nearly no observable difference in their positional amino acid usage (FIG. 6), suggesting that neural networks are effectively capturing high-dimensional patterns.

In the current study, LSTM-RNNs and CNNs were selected as the basis of our classification models, as they represent two state of the art approaches in deep learning. Other machine learning approaches such as k-nearest neighbors, random forests, and support vector machines are also well-suited at identifying complex patterns from limited input data. Furthermore, deep generative modeling methods such as variational autoencoders can also be used to explore the mutagenesis sequence space from directed evolution.

Approximately 10⁸ CDRH3 variants were in silico generated from the DMS-based combinatorial diversity and used the fully trained LSTM-RNN and CNN models to classify each sequence as a binder or non-binder. The ˜10⁸ sequence variants comprise only a subset of the potential sequence space and was chosen to minimize the computational effort, however it still represents a library size several orders of magnitude greater than what is experimentally achievable in mammalian cells. The screening capacity can be extended through script optimization and employing parallel computing on high performance clusters. Out of all variants classified, the LSTM-RNN and CNN predicted approximately 12-13% to bind the target antigen, showing exceptional agreement with the experimentally observed frequencies by flow cytometry (FIG. 14). With the exception of critical residues determined by DMS, the majority of predicted binders were substantially distant from the original Trastuzumab sequence with 80% of sequences having an edit distances of at least 6 residues. This high degree of sequence variability indicated the potential for a wide range of biomolecular properties.

Once an antibody's affinity for its target antigen is within a desirable range for efficacious biological modification, addressing other biomolecular properties becomes the focus of antibody development. With recent advances in computational predictions, a number of these properties, including viscosity, clearance, stability, specificity, solubility, and immunogenicity can be approximated from sequence information alone. With the aim of selecting antibodies with improved characteristics, the library of predicted binders was subjected to a number of these in silico approaches in order to provide a ranking structure and filtering strategy for developability (FIG. 7). After implementing these methods to remove variants with a high likelihood of having poor viscosity, clearance, or solubility, as well as those with high immunogenic potential, approximately 5,000 multi-parameter optimized antibody variants remained. More stringent or additional filters can also be applied to address other developability parameters (e.g. stability, specificity, humanization) to further reduce the sequence space down to highly developable therapeutic molecules.

Lastly, to experimentally validate the accuracy of neural networks to predict antigen specificity, we randomly selected and expressed 30 variants from the library of optimized sequences with a minimum edit distance of 5 from trastuzumab. The precision of the LSTM-RNN and CNN models were each estimated to be ˜85% (at P >0.75) according to predictions made on the test data sets. By taking the consensus between models, however, it was experimentally validated that >96% (29/30) of the antigen-predicted (and developability filtered) sequences were indeed binders. This suggests that potentially thousands of optimized lead candidates, all substantially different from the starting trastuzumab sequence, maintain a binding affinity in the range of therapeutic relevance.

The methods provided herein can be further modified to increase the stringency of selection during screening or investigation of correlations between prediction probability and affinity, which can assist in retaining high target affinities. These methods also can enable the optimization of other functional properties of therapeutic antibodies, such as pH-dependent antibody recycling or pH-dependent antigen binding. Additionally, extending this approach to other regions across the variable light and heavy chain genes, namely other CDRs, can yield deep neural networks that are able to capture long-range, complex relationships between an antibody and its target antigen. In addition, the described neural network predictions can be compared to protein structural modeling predictions.

### C. Methods

1) Mammalian Cell Culture and Transfection

Hybridoma cells were cultured and maintained according to the protocols described by

Mason et al. (2018) Nucleic Acids Research 46 (14): 7436-49. Hybridoma cells were electroporated with the 4D-Nucleofector™System (Lonza) using the SF Cell Line 4D-Nucleofector® X Kit L or X Kit S (Lonza, V4XC-2024, V4XC-2032) with the program CQ-104. Cells were prepared as follows: cells were isolated and centrifuged at 125×G for 10 minutes, washed with Opti-MEM® I Reduced Serum Medium (Thermo, 31985-062), and centrifuged again with the same parameters. The cells were resuspended in SF buffer (per kit manufacturer guidelines), after which Alt-R gRNA (IDT) and ssODN donor (IDT) were added. All experiments performed utilize constitutive expression of Cas9 from Streptococcus pyogenes (SpCas9). Transfections of 1×10⁶ and 1×10⁷ cells were performed in 100 μl, single Nucleocuvettes™ with 0.575 or 2.88 nmol Alt-R gRNA and 0.5 or 2.5 nmol ssODN donor respectively. Transfections of 2×10⁵ cells were performed in 16-well, 20 ul Nucleocuvette™ stips with 115 pmol Alt-R gRNA and 100 pmol ssODN donor.

2) Flow Cytometry Analysis and Sorting

Flow cytometry-based analysis and cell isolation were performed using the BD LSR Fortessa™ (BD Biosciences) and Sony SH800S (Sony), respectively. When labeling with fluorescently conjugated antigen or anti-IgG antibodies, cells were first washed with PBS, incubated with the labeling antibody and/or antigen for 30 minutes on ice, protected from light, washed again with PBS and then analyzed or sorted. The labeling reagents and working concentrations are described in FIGS. 23A and 23B. For cell numbers different from 10⁶, the antibody/antigen amount and incubation volume were adjusted proportionally.

3) Sample Preparation for Deep Sequencing

Sample preparation for deep sequencing was performed similar to the antibody library generation protocol of the primer extension method described previously (Menzel, et al. (2014) PloS One 9 (5): e96727). Genomic DNA was extracted from 1-5×10⁶ cells using the Purelink™ Genomic DNA Mini Kit (Thermo, K182001). All extracted genomic DNA was subjected to a first PCR step. Amplification was performed using a forward primer binding to the beginning of the VH framework region and a reverse primer specific to the intronic region immediately 3′ of the J segment. PCRs were performed with Q5® High-Fidelity DNA polymerase (NEB, M0491L) in parallel reaction volumes of 50 ml with the following cycle conditions: 98° C. for 30 seconds; 16 cycles of 98° C. for 10 sec, 70° C. for 20 sec, 72° C. for 30 sec; final extension 72° C. for 1 min; 4° C. storage. PCR products were concentrated using DNA Clean and Concentrator (Zymo, D4013) followed by 0.8×SPRIselect (Beckman Coulter, B22318) left-sided size selection. Total PCR1 product was amplified in a PCR2 step, which added extension-specific full-length Illumina adapter sequences to the amplicon library. Individual samples were Illumina-indexed by choosing from 20 different index reverse primers. Cycle conditions were as follows: 98° C. for 30 sec; 2 cycles of 98° C. for 10 sec, 40° C. for 20 sec, 72° C. for 1 min; 6 cycles of 98° C. for 10 sec, 65° C. for 20 sec, 72° C. for 1 min; 72° C. for 5 min; 4° C. storage. PCR2 products were concentrated again with DNA Clean and Concentrator and run on a 1% agarose gel. Bands of appropriate size (˜550 bp) were gel-purified using the Zymoclean™ Gel DNA Recovery kit (Zymo, D4008). Concentration of purified libraries were determined by a Nanodrop 2000c spectrophotometer and pooled at concentrations aimed at optimal read return. The quality of the final sequencing pool was verified on a fragment analyzer (Advanced Analytical Technologies) using DNF-473 Standard Sensitivity NGS fragment analysis kit. All samples passing quality control were sequenced. Antibody library pools were sequenced on the Illumina MiSeq platform using the reagent kit v3 (2×300 cycles, paired-end) with 10% PhiX control library. Base call quality of all samples was in the range of a mean Phred score of 34.

4) Bioinformatics Analysis and Graphics

The MiXCR v2.0.3 program was used to perform data pre-processing of raw FASTQ files (Bolotin et al. (2015) Nature Methods 12 (5): 380-81). Sequences were aligned to a custom germline gene reference database containing the known sequence information of the V- and J-gene regions for the variable heavy chain of the trastuzumab antibody gene. Clonotype formation by CDRH3 and error correction were performed as described by Bolotin et al. Functional clonotypes were discarded if: 1) a duplicate CDRH3 amino acid sequence arising from MiXCR uncorrected PCR errors, or 2) a clone count equal to one. Downstream analysis was performed using R v3.2.2 (Cite R Development Core Team (2008)) and Python v3.6.5 (Van Rossumet al. (2011) The Python Language Reference Manual. Network Theory). Graphics were generated using the R packages ggplot2 (Wilkinson (2011) Biometrics, found at https://doi.org/10.1111/j.1541-0420.2011.01616.x, RColorBrewer (Brewer et al. (2003) Cartography and Geographic Information Science, found at https://doi.org/10.1559/152304003100010929, and ggseqlogo (Wagih (2017) Bioinformatics 33 (22): 3645-47).

5) Calculation of Enrichment Ratios (ERs) in DMS

The ERs of a given variant was calculated according to previous methods (Fowler et al. (2010) Nature Methods 7 (9): 741-46). Clonal frequencies of variants enriched for antigen specificity by FACS, f_{i,Ag+}, were divided by the clonal frequencies of the variants present in the original library, f_{i,Ab+}, according to Equation 1, above.

A minimum value of −2 was designated to variants with log[ER] values less than or equal −2 and variants not present in the dataset were disregarded in the calculation. A clone was defined based on the exact amino acid sequence of the CDRH3.

6) Redesign of Trastuzumab in Rosetta for Diversity of Sequences

The Rosetta program (Leaver-Fay et al.) was used to redesign the trastuzumab antibody in complex with the extracellular domain of HER2 (PDB id: 1N8Z) (Cho et al.). Ten residues in the CDRH3 loop of trastuzumab (residues 98-108 of the heavy chain) were allowed to mutate to any natural amino acid, while all other residues were allowed to change rotameric conformation. A RosettaScript invoked the PackRotamersMover, a stochastic MonteCarlo algorithm, to optimize the sequence of the antibody to CDRH3 according to the Rosetta energy function, followed by backbone minimization. Energies were computed using Rosetta's ddG filter. Rosetta was run to generate 5000 sequences stochastically, and this resulted in 48 sequences. Rosetta's output files were processed using RS-Toolbox (Bonet et al., 2019).

7) Classification of Experimentally-Determined Sequences in Rosetta

Each of the 11,300 binding and 27,539 non-binding sequences from the combinatorial library were modelled in Rosetta. For each experimentally-determined binding or non-binding sequence, the structure of the HER2:trastuzumab complex was used as input and the residues diverging from the wildtype were mutated using the PackRotamersMover in RosettaScripts (Fleishman et al.). The backbone and the side chains were minimized with Rosetta's MinMover after the sequence was modeled to optimized intra- and inter-chain contacts. Rosetta's predicted interface score (ddG) was used as the relative classification score.

8) Codon Selection for Rational Library Design

Codon selection for rational library design was based off the equation provided by Mason et al. (2018) Nucleic Acids Research 46 (14): 7436-49, (Equation 2). Residues identified in DMS analysis to have a positive enrichment (ER >1, or log[ER]>0) were normalized according to their enrichment ratios and were converted to theoretical frequencies. Degenerate codon schemes were then selected which most closely reflect these frequencies as calculated by the mean squared error between the degenerate codon and the target frequencies.

In certain instances, if the selected degenerate codon did not represent desirable amino acid frequencies or contained undesirable amino acids, a mixture of degenerate codons was selected and pooled together to achieve better coverage of the functional sequence space.

9) Machine Learning Model Construction

Machine learning models were built in Python v3.6.5. K-nearest neighbor models and support vector machine models were built using the Scikit-learn libraries. Artificial neural networks, LSTM-RNNs, and CNNs were built using the Keras Sequential model as a wrapper for TensorFlow. Model architecture and hyperparameters were optimized by performing a grid search of relevant variables for a given model. These variables include nodes per layer, activation function(s), optimizer, loss function, dropout rate, batch size, number of epochs, number of filters, kernel size, stride length, and pool size. Grid searches were performed by implementing a k-fold cross validation of the data set.

10) Machine Learning Model Training and Testing

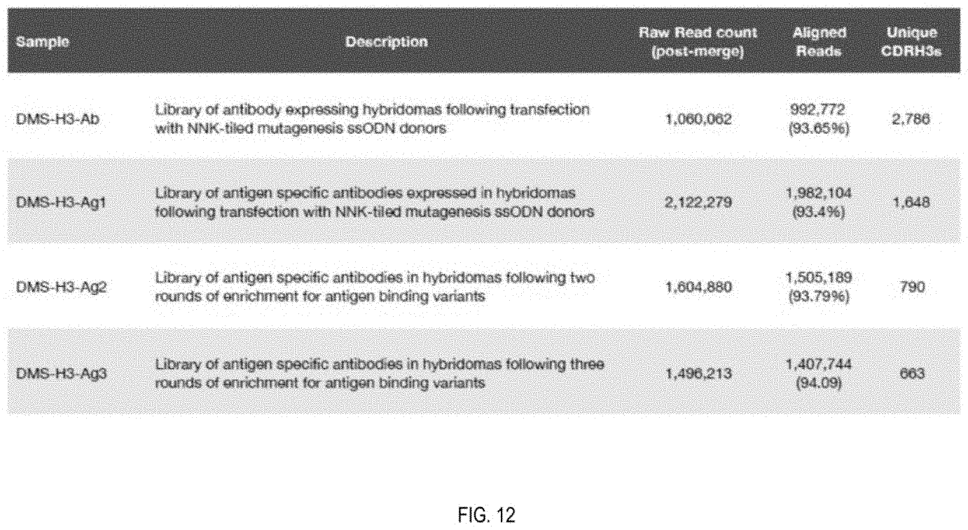

Data sets for antibody expressing, non-binding, and binding sequences (Sequencing statistics: FIGS. 12 and 13) were aggregated to form a single, binding/non-binding data set where antibody expressing sequences were classified as non-binders, unless also identified among the binding sequences. Sequences from one round of antigen enrichment were excluded from the training data set. The complete, aggregated data set was then randomly arranged and appropriate class labeled sequences were removed to achieve the desired classification ratio of binders to non-binders (50/50, 20/80, 10/90, and non-adjusted). The class adjusted data set was further split into a training set (70%), and two testing sets (15% each), where one test set reflected the classification ratio observed for training and the other reflected a classification ratio of approximately 10/90 to resemble the physiological expected frequency of binders.

11) Sequence Similarity and Model Attribution Analysis of Predicted Variants

Sequence similarity networks of sequences predicted to be antigen positive and antigen negative were constructed for Levenshtein Distance 1-6 were constructed using the igraph R package v1.2.4 (Csardi and Nepusz 2006). The resulting networks were analyzed with respect to their overall connectivity, the composition of their largest clusters and the overall degree distribution between the classes.

The Integrated Gradients technique (Sundararajan et al. 2017) was used to assess the relative attribution of each feature of a given input sequence towards the final prediction score. First, a baseline was obtained by zeroing out the input vector and the path integral of the gradients from baseline to the input vector was then approximated with a step size of 100. Integrated gradients were visualized as sequence logos. Sequence logos were created by the python module Logomaker (Tareen and Kinney 2019).

12) In Silico Sequence Classification and Sequence Parameters

All possible combinations of amino acids present in the DMS-based combinatorial mutagenesis libraries were used to calculate the total theoretical sequence space of 7.17×10⁸. 7.2×10⁷ sequence variants were generated in silico by taking all possible combinations of the amino acids used per position in the combinatorial mutagenesis library designed from the DMS data following three rounds of enrichment for antigen binding variants; Alanine was also selected to be included at position 103. All in silico sequences were then classified as a binder or non-binder by the trained LSTM-RNN and CNN models. Sequences were selected for further analysis if they were classified in both models with a prediction probability (P) of more than 0.75.

The Fv net charge and Fv charge symmetry parameter (FvCSP) were calculated as described by Sharma et al. Briefly, the net charge was determined by first solving the Henderson-Hasselbalch equation for each residue at a specified pH (here 5.5) with known amino acid pKas. The sum across all residues for both the VL and VH was then calculated as the Fv net charge. The FvCSP was calculated by taking the product of the VL and VH net charges. The hydrophobicity index (HI) was also calculated as described by Sharma et al., according to the following equation: HI=−(ΣniEi/ΣnjEj). E represents the Eisenberg value of an amino acid, n is the number of an amino acid, and i and j are hydrophobic and hydrophilic residues respectively.

The protein solubility score was determined for each, full-length CDRH3 sequence (15 a.a.) padded with 10 amino acids on both the 5′ and 3′ ends (35 a.a.) by the CamSol method at pH 7.0.

The binding affinities for a reference set of 26 HLA alleles were determined for each 15-mer contained within the 10 amino acid padded CDRH3 sequence (35 a.a.) by NetMHCIIpan 3.2. The output provides for each 15-mer a predicted affinity in nM and the % Rank which reflects the 15-mer's affinity compared to a set of random natural peptides. The % Rank measure is unaffected by the bias of certain molecules against stronger or weaker affinities and is used to classify peptides as weak or strong binders towards the specified MHC Class II allele. The minimum % Rank, the number of 15-mers with % Rank less than 10 (classification of weak binder), and the average % Rank were calculated across all 21 15-mers for a single CDRH3 sequence across all 26 HLA alleles.

Overall developability improvement of the antibody sequences was determined by first normalizing the FvCSP, CamSol score, and average NetMHCII % Rank according to the range of values observed in the remaining sequences post-filtering. The normalized CamSol protein solubility score was then weighted by a factor of 2 for its importance in determining developability. Lastly, the mean across these three parameters was taken to produce the overall developability improvement score. Since the sequences were filtered with the calculated values for trastuzumab, trastuzumab would have an overall developability improvement equal to 0.

$$Overall developability = \frac{1}{3} ( ( \frac{FvCSP - min ( FvCSP )}{max ( FvCSP ) - min ( FvCSP )} ) + 2 * ( \frac{CamSol - min ( CamSol )}{max ( CamSol ) - min ( CamSol )} ) + ( \frac{avgNetMHC - min ( avgNetMHC )}{max ( avgNetMHC ) - min ( avgNetMHC )} ) ) \qquad ( 3 )$$

13) Expression and Affinity Measurements by Biolayer Interferometry

Monoclonal populations of the individual variants were isolated by performing a single-cell sort. Following expansion, supernatant for all variants was collected and filtered through a 0.20 μm filter (Sartorius, 16534-K). Affinity measurements were then performed on an Octet RED96e (FortéBio) with the following parameters. Anti-human capture sensors (FortéBio, 18-5060) were hydrated in conditioned media diluted 1 in 2 with kinetics buffer (FortéBio, 18-1105) for at least 10 minutes before conditioning through 4 cycles of regeneration consisting of 10 seconds incubation in 10 mM glycine, pH 1.52 and 10 seconds in kinetics buffer. Conditioned sensors were then loaded with 0 ug/mL (reference sensor), 10 ug/mL trastuzumab (reference sample), or hybridoma supernatant (approximately 20 μg/mL) diluted 1 in 2 with kinetics buffer followed by blocking with mouse IgG (Rockland, 010-0102) at 50 μg/mL in kinetics buffer. After blocking, loaded sensors were equilibrated in kinetics buffer and incubated with either 5 nM or 25 nM HER2 protein (Sigma-Aldrich, SRP6405-50UG). Lastly, sensors were incubated kinetics buffer to allow antigen dissociation. Antibody expression and kinetics analysis was performed in analysis software Data Analysis HT v11.0.0.50.

14) Thermal Stability Measurements by Fluorescence

Monoclonal antibodies of the individual variants were purified by Protein A column chromatography from the supernatant of their respective monoclonal cell line and eluted into 200 mM sodium dihydrogen phosphate, 140 mM sodium chloride, pH 2.5. Protein purity was verified by SDS-PAGE prior to downstream analysis. Purified antibody was loaded into Unchained Lab's UNcle instrument and static light scattering (SLS) and fluorescence measurements were taken while exposing the antibody to a thermal ramp from 20° C. to 95° C. at a rate of 0.5° C. per minute. The melting temperature (Tm) is identified as the inflection point of the first derivative of the barycentric mean (BCM) as a function of the temperature.

15) Immunogenicity Risk Assessment by T-Cell Proliferation Assay

Immunogenicity risk was assessed by ProImmune's ProMap® T Cell Proliferation assay. Briefly, 15-mer peptides for specified variant sequences were synthesized and used for the in vitro assessment of potential antigenicity. Each 15-mer peptide is pulsed into donor antigen presenting cells which are then co-cultured with the donor's CD4+ T cells. CD4+ T cell proliferation is then measured by flow cytometry. The assay was performed by testing the peptides against 20 healthy donor cell samples. Donor cell samples were CD8-depleted prior to use, to eliminate CD8+ responses from the analysis. Detection of proliferation of CD4+ T cells was performed by labeling cells with CFSE and co-staining with anti-human CD4 antibody.

Where technical features in the drawings, detailed description or any claim are followed by reference signs, the reference signs have been included to increase the intelligibility of the drawings, detailed description, and claims. Accordingly, neither the reference signs nor their absence has any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

As used herein, the term “about” and “substantially” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation or embodiment, and references to “an implementation,” “some implementations,” “one implementation” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

The indefinite articles “a” and “an,” as used herein in the specification and in the claims, unless clearly indicated to the contrary, should be understood to mean “at least one.”

References to “or” may be construed as inclusive so that any terms described using “or” may indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

The terminology used in the description herein is for the purpose of describing particular embodiments only and is not intended to be limiting of the invention. All publications, patent applications, patents and other references mentioned herein are incorporated by reference in their entirety.

1. A method, comprising:
providing an input amino acid sequence that represents an antigen binding portion of an antigen binding molecule;
generating a first training data set comprising a first plurality of variant sequences, each of the first plurality of variant sequences comprising a single site mutation in the input amino acid sequence of the antigen binding molecule;
generating a second training data set comprising a second plurality of sequences, each of the second plurality of sequences comprising a plurality of variants at positions based on enrichment scores of the first training data set comprising the first plurality of variant sequences;
providing the second training data set to a classification engine comprising a first machine learning model to generate a plurality of weights and biases for the first machine learning model;
determining, by the classification engine based on the plurality of weights and bias for the first machine learning model, a first affinity binding score for a proposed amino acid sequence to an antigen; and
selecting the proposed amino acid sequence for expression based on the first affinity binding score satisfying a threshold.

2. The method of claim 1, wherein the antigen binding molecule comprises an antibody, or an antigen binding fragment thereof.

3. The method of claim 1, wherein the antigen binding molecule comprises a chimeric antigen receptor.

4. The method of claim 1, comprising:
determining, by the classification engine, a second affinity binding score for the proposed amino acid sequence using a second machine learning model of the classification engine; and
selecting the proposed amino acid sequence for expression based on the first affinity binding score and the second affinity binding score satisfying the threshold.

5. The method of claim 1, comprising:
determining, by the classification engine, an affinity binding score for each of a plurality of proposed amino acid sequences;
determining, by a candidate selection engine, one or more parameters for each of the plurality of proposed amino acid sequences; and
selecting, by the candidate selection engine, candidate variants from the plurality of proposed amino acid sequences based on the affinity binding score and the one or more parameters for each of the plurality of proposed amino acid sequences.

6. The method of claim 5, wherein the candidate selection engine selects only the variants that were classified with a predetermined confidence or probability level.

7. The method of claim 6, wherein the predetermined confidence or probability level is above 0.5.

8. The method of claim 5, wherein the candidate selection engine selects variants based on the proposed amino acid sequence satisfying a threshold for at least of one of the one or more additional parameters.

9. The method of claim 5, wherein the candidate selection engine selects variants based on the proposed amino acid sequence satisfying a threshold for each of the one or more additional parameters.

10. The method of claim 9, wherein the threshold for each of the one or more additional parameters includes a value threshold.

11. The method of claim 9, wherein the threshold for each of the one or more additional parameters includes a variable or relative threshold.

12. The method of claim 9, wherein the threshold for one or more of the additional parameters is a parameter value in the top 5% or top 10%.

13. The method of claim 9, wherein the threshold for one or more of the additional parameters is based on a number of standard deviations above the average for the one or more parameters.

14. The method of claim 5, wherein the one or more parameters comprise viscosity values, solubility values, stability values, pharmacokinetic values, and/or immunogenicity values.

15. The method of claim 5, wherein the one or more parameters comprise a Levenshtein distance value.

16. The method of claim 5, wherein the one or more parameters comprise charge value.

17. The method of claim 16, wherein the charge value is a variable fragment (Fv) charge value.

18. The method of claim 17, wherein the Fv charge value is between about 0 and about 6.2.

19. The method of claim 16, wherein the charge value is a variable fragment charge symmetry parameter (FvCSP) value.

20.-87. (canceled)

88. A system comprising one or more processors and a memory storing processor-executable instructions, the one or more processors execute the processor-executable instructions to:
receive an input amino acid sequence that represents an antigen binding portion of an antibody;
receive a first training data set comprising a first plurality of variant sequences, each of the first plurality of variant sequences comprising a single site mutation in the input amino acid sequence of the antibody;
receive a second training data set comprising a second plurality of sequences, each of the second plurality of sequences comprising a plurality of variants at positions based on enrichment scores of the first training data set comprising the first plurality of variant sequences;
provide the second training data set to a classification engine comprising a first machine learning model to generate a plurality of weights and bias for the first machine learning model;
determine, based on the plurality of weights and bias for the first machine learning model, a first affinity binding score for a proposed amino acid sequence to an antigen; and
select the proposed amino acid sequence for expression based on the first affinity binding score satisfying a threshold.

89.-123. (canceled)


SEQUENCE LISTING (134 sequences)

SEQ ID NO: 1 - protein, 10 aa, Artificial Sequence
  source: /note=“Description of Artificial Sequence Synthetic peptide”
WGGDGFYAMD

SEQ ID NO: 2 - DNA, 30 bp, Artificial Sequence
  source: /note=“Description of Artificial Sequence Synthetic oligonucleotide”
  modified_base (5)..(5): a, c, t, g, unknown or other
  modified_base (8)..(8): a, c, t, g, unknown or other
  modified_base (10)..(11): a, c, t, g, unknown or other
twcvnbvnsnncrghwtstacvhghwcvhb

SEQ ID NO: 3 - DNA, 52 bp, Artificial Sequence
  source: /note=“Description of Artificial Sequence Synthetic oligonucleotide”
atcggacttaagcttggcgcactagccgcattttcagggcaaagcatcatta

SEQ ID NO: 4 - DNA, 45 bp, Artificial Sequence
  source: /note=“Description of Artificial Sequence Synthetic oligonucleotide”
tgttcgcgatggggaggtgatggattttatgcaatggattactgg

SEQ ID NO: 5 - protein, 15 aa, Artificial Sequence
  source: /note=“Description of Artificial Sequence Synthetic peptide”
CSRWGGDGFYAMDYW

SEQ ID NO: 6 - DNA, 45 bp, Artificial Sequence
  source: /note=“Description of Artificial Sequence Synthetic oligonucleotide”
  modified_base (10)..(11): a, c, t, g, unknown or other
tgttcgcgannkggaggtgatggattttatgcaatggattactgg

SEQ ID NO: 7 - protein, 15 aa, Artificial Sequence
  source: /note=“Description of Artificial Sequence Synthetic peptide”
  MOD_RES (4)..(4): Any amino acid
CSRXGGDGFYAMDYW

SEQ ID NO: 8 - DNA, 45 bp, Artificial Sequence
  source: /note=“Description of Artificial Sequence Synthetic oligonucleotide”
  modified_base (13)..(14): a, c, t, g, unknown or other
tgttcgcgatggnnkggtgatggattttatgcaatggattactgg

SEQ ID NO: 9 - protein, 15 aa, Artificial Sequence
  source: /note=“Description of Artificial Sequence Synthetic peptide”
  MOD_RES (5)..(5): Any amino acid
CSRWXGDGFYAMDYW

SEQ ID NO: 10 - DNA, 45 bp, Artificial Sequence
  source: /note=“Description of Artificial Sequence Synthetic oligonucleotide”
  modified_base (16)..(17): a, c, t, g, unknown or other
tgttcgcgatggggannkgatggattttatgcaatggattactgg

SEQ ID NO: 11 - protein, 15 aa, Artificial Sequence
  source: /note=“Description of Artificial Sequence Synthetic peptide”
  MOD_RES (6)..(6): Any amino acid
CSRWGXDGFYAMDYW

SEQ ID NO: 12 - DNA, 45 bp, Artificial Sequence
  source: /note=“Description of Artificial Sequence Synthetic oligonucleotide”
  modified_base (19)..(20): a, c, t, g, unknown or other
tgttcgcgatggggaggtnnkggattttatgcaatggattactgg

SEQ ID NO: 13 - protein, 15 aa, Artificial Sequence
  source: /note=“Description of Artificial Sequence Synthetic peptide”
  MOD_RES (7)..(7): Any amino acid
CSRWGGXGFYAMDYW

SEQ ID NO: 14 - DNA, 45 bp, Artificial Sequence
  source: /note=“Description of Artificial Sequence Synthetic oligonucleotide”
  modified_base (22)..(23): a, c, t, g, unknown or other
tgttcgcgatggggaggtgatnnkttttatgcaatggattactgg

SEQ ID NO: 15 - protein, 15 aa, Artificial Sequence
  source: /note=“Description of Artificial Sequence Synthetic peptide”
  MOD_RES (8)..(8): Any amino acid
CSRWGGDXFYAMDYW

SEQ ID NO: 16 - DNA, 45 bp, Artificial Sequence
  source: /note=“Description of Artificial Sequence Synthetic oligonucleotide”
  modified_base (25)..(26): a, c, t, g, unknown or other
tgttcgcgatggggaggtgatggannktatgcaatggattactgg

SEQ ID NO: 17 - protein, 15 aa, Artificial Sequence
  source: /note=“Description of Artificial Sequence Synthetic peptide”
  MOD_RES (9)..(9): Any amino acid
CSRWGGDGXYAMDYW

SEQ ID NO: 18 - DNA, 45 bp, Artificial Sequence
  source: /note=“Description of Artificial Sequence Synthetic oligonucleotide”
  modified_base (28)..(29): a, c, t, g, unknown or other
tgttcgcgatggggaggtgatggatttnnkgcaatggattactgg

SEQ ID NO: 19 - protein, 15 aa, Artificial Sequence
  source: /note=“Description of Artificial Sequence Synthetic peptide”
  MOD_RES (10)..(10): Any amino acid
CSRWGGDGFXAMDYW

SEQ ID NO: 20 - DNA, 45 bp, Artificial Sequence
  source: /note=“Description of Artificial Sequence Synthetic oligonucleotide”
  modified_base (31)..(32): a, c, t, g, unknown or other
tgttcgcgatggggaggtgatggattttatnnkatggattactgg

SEQ ID NO: 21 - protein, 15 aa, Artificial Sequence
  source: /note=“Description of Artificial Sequence Synthetic peptide”
  MOD_RES (11)..(11): Any amino acid
CSRWGGDGFYXMDYW

SEQ ID NO: 22 - DNA, 45 bp, Artificial Sequence
  source: /note=“Description of Artificial Sequence Synthetic oligonucleotide”
  modified_base (34)..(35): a, c, t, g, unknown or other
tgttcgcgatggggaggtgatggattttatgcannkgattactgg

SEQ ID NO: 23 - protein, 15 aa, Artificial Sequence
  source: /note=“Description of Artificial Sequence Synthetic peptide”
  MOD_RES (12)..(12): Any amino acid
CSRWGGDGFYAXDYW

SEQ ID NO: 24 - DNA, 45 bp, Artificial Sequence
  source: /note=“Description of Artificial Sequence Synthetic oligonucleotide”
  modified_base (37)..(38): a, c, t, g, unknown or other
tgttcgcgatggggaggtgatggattttatgcaatgnnktactgg

SEQ ID NO: 25 - protein, 15 aa, Artificial Sequence
  source: /note=“Description of Artificial Sequence Synthetic peptide”
  MOD_RES (13)..(13): Any amino acid
CSRWGGDGFYAMXYW

SEQ ID NO: 26 - DNA, 30 bp, Artificial Sequence
  source: /note=“Description of Artificial Sequence Synthetic oligonucleotide”
  variation (1)..(3): /replace=“tgg”
  modified_base (5)..(5): a, c, t, g, unknown or other
  modified_base (8)..(8): a, c, t, g, unknown or other
  modified_base (10)..(11): a, c, t, g, unknown or other
  variation (16)..(18): /replace=“mtg”
  misc_feature (1)..(30): /note=“Variant nucleotides given in the sequence have no preference with respect to those in the annotations for variant positions”
twcvnbvnsnncrgyttctacvhghwcvhb

SEQ ID NO: 27 - DNA, 33 bp, Artificial Sequence
  source: /note=“Description of Artificial Sequence Synthetic oligonucleotide”
tgtgcaagatcctctgggttcgattattggggc

SEQ ID NO: 28 - protein, 11 aa, Artificial Sequence
  source: /note=“Description of Artificial Sequence Synthetic peptide”
CARSSGFDYWG

SEQ ID NO: 29 - protein, 15 aa, Artificial Sequence
  source: /note=“Description of Artificial Sequence Synthetic peptide”
CSRWSEPGFYTNDYW

SEQ ID NO: 30 - protein, 15 aa, Artificial Sequence
  source: /note=“Description of Artificial Sequence Synthetic peptide”
CSRWMETGFYTHDYW

SEQ ID NO: 31 - protein, 15 aa, Artificial Sequence
  source: /note=“Description of Artificial Sequence Synthetic peptide”
CSRWKGDGFYEHDYW

SEQ ID NO: 32 - protein, 15 aa, Artificial Sequence
  source: /note=“Description of Artificial Sequence Synthetic peptide”
CSRWPAPGFYTNDYW

SEQ ID NO: 33 - protein, 15 aa, Artificial Sequence
  source: /note=“Description of Artificial Sequence Synthetic peptide”
CSRWPGPGMYTNDYW

SEQ ID NO: 34 - protein, 15 aa, Artificial Sequence
  source: /note=“Description of Artificial Sequence Synthetic peptide”
CSRWPGPGMYANDYW

SEQ ID NO: 35 - protein, 15 aa, Artificial Sequence
  source: /note=“Description of Artificial Sequence Synthetic peptide”
CSRWRGDGFYEHDYW

SEQ ID NO: 36 - protein, 15 aa, Artificial Sequence
  source: /note=“Description of Artificial Sequence Synthetic peptide”
CSRWQRGGFYVNDYW

SEQ ID NO: 37 - protein, 15 aa, Artificial Sequence
  source: /note=“Description of Artificial Sequence Synthetic peptide”
CSRWGIHSFYEHDYW

SEQ ID NO: 38 - protein, 15 aa, Artificial Sequence
  source: /note=“Description of Artificial Sequence Synthetic peptide”
CSRWREPGFYEHDYW

SEQ ID NO: 39 - protein, 15 aa, Artificial Sequence
  source: /note=“Description of Artificial Sequence Synthetic peptide”
CSRWRDRGFYEYDYW

SEQ ID NO: 40 - protein, 15 aa, Artificial Sequence
  source: /note=“Description of Artificial Sequence Synthetic peptide”
CSRWGQAGFYEHDYW

SEQ ID NO: 41 - protein, 15 aa, Artificial Sequence
  source: /note=“Description of Artificial Sequence Synthetic peptide”
CSRWKEPGFYEHDYW

SEQ ID NO: 42 - protein, 15 aa, Artificial Sequence
  source: /note=“Description of Artificial Sequence Synthetic peptide”
CSRWKDPGFYEHDYW

SEQ ID NO: 43 - protein, 15 aa, Artificial Sequence
  source: /note=“Description of Artificial Sequence Synthetic peptide”
CSRWGACGFYENDYW

SEQ ID NO: 44 - protein, 15 aa, Artificial Sequence
  source: /note=“Description of Artificial Sequence Synthetic peptide”
CSRYRAVGFYTNDYW

SEQ ID NO: 45 - protein, 15 aa, Artificial Sequence
  source: /note=“Description of Artificial Sequence Synthetic peptide”
CSRYGPPGMYTNDYW

SEQ ID NO: 46 - protein, 15 aa, Artificial Sequence
  source: /note=“Description of Artificial Sequence Synthetic peptide”
CSRWRDDGLYAHDYW

SEQ ID NO: 47 - protein, 15 aa, Artificial Sequence
  source: /note=“Description of Artificial Sequence Synthetic peptide”
CSRWNDRGFYEHDYW

SEQ ID NO: 48 - protein, 15 aa, Artificial Sequence
  source: /note=“Description of Artificial Sequence Synthetic peptide”
CSRWNPHGLYVNDYW

SEQ ID NO: 49 - protein, 15 aa, Artificial Sequence
  source: /note=“Description of Artificial Sequence Synthetic peptide”
CSRWPGGGMYANDYW

SEQ ID NO: 50 - protein, 15 aa, Artificial Sequence
  source: /note=“Description of Artificial Sequence Synthetic peptide”
CSRYGEYGMYVNKYW

SEQ ID NO: 51 - protein, 15 aa, Artificial Sequence
  source: /note=“Description of Artificial Sequence Synthetic peptide”
CSRWAPYGLYAHDYW

SEQ ID NO: 52 - protein, 15 aa, Artificial Sequence
  source: /note=“Description of Artificial Sequence Synthetic peptide”
CSRWGGPGMYTNDYW

SEQ ID NO: 53 - protein, 15 aa, Artificial Sequence
  source: /note=“Description of Artificial Sequence Synthetic peptide”
CSRWRDPGFYEHDYW

SEQ ID NO: 54 - protein, 15 aa, Artificial Sequence
  source: /note=“Description of Artificial Sequence Synthetic peptide”
CSRWGPSGLYVNDYW

SEQ ID NO: 55 - protein, 15 aa, Artificial Sequence
  source: /note=“Description of Artificial Sequence Synthetic peptide”
CSRWKQDGFYEHDYW

SEQ ID NO: 56 - protein, 15 aa, Artificial Sequence
  source: /note=“Description of Artificial Sequence Synthetic peptide”
CSRYGMRGLYAYEYW

SEQ ID NO: 57 - protein, 15 aa, Artificial Sequence
  source: /note=“Description of Artificial Sequence Synthetic peptide”
CSRYGGPGMYTNDYW

SEQ ID NO: 58 - protein, 15 aa, Artificial Sequence
  source: /note=“Description of Artificial Sequence Synthetic peptide”
CSRYGGRGMYQLEYW

SEQ ID NO: 59 - protein, 15 aa, Artificial Sequence
  source: /note=“Description of Artificial Sequence Synthetic peptide”
CSRYGGDGMYQHDYW

SEQ ID NO: 60 - protein, 15 aa, Artificial Sequence
  source: /note=“Description of Artificial Sequence Synthetic peptide”
CSRYGGTGMYEHDYW

SEQ ID NO: 61 - protein, 15 aa, Artificial Sequence
  source: /note=“Description of Artificial Sequence Synthetic peptide”
CSRWGSNGMYVNDYW

SEQ ID NO: 62 - protein, 15 aa, Artificial Sequence
  source: /note=“Description of Artificial Sequence Synthetic peptide”
CSRWEEYGLYVHDYW

SEQ ID NO: 63 - protein, 15 aa, Artificial Sequence
  source: /note=“Description of Artificial Sequence Synthetic peptide”
CSRYGNGGMYANDYW

SEQ ID NO: 64 - protein, 15 aa, Artificial Sequence
  source: /note=“Description of Artificial Sequence Synthetic peptide”
CSRWAMLGMYAHDYW

SEQ ID NO: 65 - protein, 15 aa, Artificial Sequence
  source: /note=“Description of Artificial Sequence Synthetic peptide”
CSRWPKVGLYTNDYW

SEQ ID NO: 66 - protein, 15 aa, Artificial Sequence
  source: /note=“Description of Artificial Sequence Synthetic peptide”
CSRYGPGGMYEHDYW

SEQ ID NO: 67 - protein, 15 aa, Artificial Sequence
  source: /note=“Description of Artificial Sequence Synthetic peptide”
CSRWGGPGMYEHDYW

SEQ ID NO: 68 - protein, 15 aa, Artificial Sequence
  source: /note=“Description of Artificial Sequence Synthetic peptide”
CSRYRGDGFYEHDYW

SEQ ID NO: 69 - protein, 15 aa, Artificial Sequence
  source: /note=“Description of Artificial Sequence Synthetic peptide”
CSRYGGPGLYANDYW

SEQ ID NO: 70 - protein, 15 aa, Artificial Sequence
  source: /note=“Description of Artificial Sequence Synthetic peptide”
CSRYGGPGMYQNDYW

SEQ ID NO: 71 - protein, 15 aa, Artificial Sequence
  source: /note=“Description of Artificial Sequence Synthetic peptide”
CSRWPQPGMYANDYW

SEQ ID NO: 72 - protein, 15 aa, Artificial Sequence
  source: /note=“Description of Artificial Sequence Synthetic peptide”
CSRYGGPGMYEHDYW

SEQ ID NO: 73 - protein, 15 aa, Artificial Sequence
  source: /note=“Description of Artificial Sequence Synthetic peptide”
CSRWQGIGLYELDYW

SEQ ID NO: 74 - protein, 15 aa, Artificial Sequence
  source: /note=“Description of Artificial Sequence Synthetic peptide”
CSRWQEAAFYAHDYW

SEQ ID NO: 75 - protein, 15 aa, Artificial Sequence
  source: /note=“Description of Artificial Sequence Synthetic peptide”
CSRYGDPGMYQHDYW

SEQ ID NO: 76 - protein, 15 aa, Artificial Sequence
  source: /note=“Description of Artificial Sequence Synthetic peptide”
CSRYGGPGLYTNDYW

SEQ ID NO: 77 - protein, 15 aa, Artificial Sequence
  source: /note=“Description of Artificial Sequence Synthetic peptide”
CSRWSDSGMYAHDYW

SEQ ID NO: 78 - protein, 15 aa, Artificial Sequence
  source: /note=“Description of Artificial Sequence Synthetic peptide”
CSRWNQLGMYVNDYW

SEQ ID NO: 79 - protein, 15 aa, Artificial Sequence
  source: /note=“Description of Artificial Sequence Synthetic peptide”
CSRYAGPGMYTNQYW

SEQ ID NO: 80 - protein, 15 aa, Artificial Sequence
  source: /note=“Description of Artificial Sequence Synthetic peptide”
CSRYGPGGLYTNDYW

SEQ ID NO: 81 - protein, 15 aa, Artificial Sequence
  source: /note=“Description of Artificial Sequence Synthetic peptide”
CSRYGGPGLYEHDYW

SEQ ID NO: 82 - protein, 15 aa, Artificial Sequence
  source: /note=“Description of Artificial Sequence Synthetic peptide”
CSRWAEAGMYEFDYW

SEQ ID NO: 83 - protein, 15 aa, Artificial Sequence
  source: /note=“Description of Artificial Sequence Synthetic peptide”
CSRYSMPGMYTNAYW

SEQ ID NO: 84 - protein, 15 aa, Artificial Sequence
  source: /note=“Description of Artificial Sequence Synthetic peptide”
CSRWGNPGMYANDYW

SEQ ID NO: 85 - protein, 15 aa, Artificial Sequence
  source: /note=“Description of Artificial Sequence Synthetic peptide”
CSRWRHDGFYENDYW

SEQ ID NO: 86 - protein, 15 aa, Artificial Sequence
  source: /note=“Description of Artificial Sequence Synthetic peptide”
CSRWGSFGLYTFDYW

SEQ ID NO: 87 - protein, 15 aa, Artificial Sequence
  source: /note=“Description of Artificial Sequence Synthetic peptide”
CSRYGPGGMYANDYW

SEQ ID NO: 88 - protein, 15 aa, Artificial Sequence
  source: /note=“Description of Artificial Sequence Synthetic peptide”
CSRWPMCGMYTHDYW

SEQ ID NO: 89 - protein, 15 aa, Artificial Sequence
  source: /note=“Description of Artificial Sequence Synthetic peptide”
CSRWGIVSFYENDYW

SEQ ID NO: 90 - protein, 15 aa, Artificial Sequence
  source: /note=“Description of Artificial Sequence Synthetic peptide”
CSRWGNGGMYANDYW

SEQ ID NO: 91 - protein, 15 aa, Artificial Sequence
  source: /note=“Description of Artificial Sequence Synthetic peptide”
CSRYGERGFYEHDYW

SEQ ID NO: 92 - protein, 15 aa, Artificial Sequence
  source: /note=“Description of Artificial Sequence Synthetic peptide”
CSRYGGNGMYTNDYW

SEQ ID NO: 93 - protein, 15 aa, Artificial Sequence
  source: /note=“Description of Artificial Sequence Synthetic peptide”
CSRYGSPGMYTNDYW

SEQ ID NO: 94 - protein, 15 aa, Artificial Sequence
  source: /note=“Description of Artificial Sequence Synthetic peptide”
CSRYGQPGMYTNDYW

SEQ ID NO: 95 - protein, 15 aa, Artificial Sequence
  source: /note=“Description of Artificial Sequence Synthetic peptide”
CSRYGEPGMYQHDYW

SEQ ID NO: 96 - protein, 15 aa, Artificial Sequence
  source: /note=“Description of Artificial Sequence Synthetic peptide”
CSRYGDAGMYALKYW

SEQ ID NO: 97 - protein, 15 aa, Artificial Sequence
  source: /note=“Description of Artificial Sequence Synthetic peptide”
CSRWGGNGMYANDYW

SEQ ID NO: 98 - protein, 15 aa, Artificial Sequence
  source: /note=“Description of Artificial Sequence Synthetic peptide”
CSRYASAGMYTHDYW

SEQ ID NO: 99 - protein, 15 aa, Artificial Sequence
  source: /note=“Description of Artificial Sequence Synthetic peptide”
CSRYGPTGMYQNDYW

SEQ ID NO: 100 - protein, 15 aa, Artificial Sequence
  source: /note=“Description of Artificial Sequence Synthetic peptide”
CSRYGDRGFYEHDYW

SEQ ID NO: 101 - protein, 15 aa, Artificial Sequence
  source: /note=“Description of Artificial Sequence Synthetic peptide”
CSRYGTPGMYQNDYW

SEQ ID NO: 102 - protein, 15 aa, Artificial Sequence
  source: /note=“Description of Artificial Sequence Synthetic peptide”
CSRYGPNGLYANDYW

SEQ ID NO: 103 - protein, 15 aa, Artificial Sequence
  source: /note=“Description of Artificial Sequence Synthetic peptide”
CSRYGGNGMYQNDYW

SEQ ID NO: 104 - protein, 15 aa, Artificial Sequence
  source: /note=“Description of Artificial Sequence Synthetic peptide”
CSRYGNPGMYQNDYW

SEQ ID NO: 105 - protein, 15 aa, Artificial Sequence
  source: /note=“Description of Artificial Sequence Synthetic peptide”
CSRYGSGGLYEHDYW

SEQ ID NO: 106 - protein, 15 aa, Artificial Sequence
  source: /note=“Description of Artificial Sequence Synthetic peptide”
CSRYGARGFYQNDYW

SEQ ID NO: 107 - protein, 15 aa, Artificial Sequence
  source: /note=“Description of Artificial Sequence Synthetic peptide”
CSRWSESGPYTHDYW

SEQ ID NO: 108 - protein, 15 aa, Artificial Sequence
  source: /note=“Description of Artificial Sequence Synthetic peptide”
CSRYGQPGMYANDYW

SEQ ID NO: 109 - protein, 15 aa, Artificial Sequence
  source: /note=“Description of Artificial Sequence Synthetic peptide”
CSRYSGPGMYANDYW

SEQ ID NO: 110 - protein, 15 aa, Artificial Sequence
  source: /note=“Description of Artificial Sequence Synthetic peptide”
CSRWNSDGLYAYEYW

SEQ ID NO: 111 - protein, 15 aa, Artificial Sequence
  source: /note=“Description of Artificial Sequence Synthetic peptide”
CSRYGSNGLYANQYW

SEQ ID NO: 112 - protein, 15 aa, Artificial Sequence
  source: /note=“Description of Artificial Sequence Synthetic peptide”
CSRYGSNGMYQNDYW

SEQ ID NO: 113 - protein, 15 aa, Artificial Sequence
  source: /note=“Description of Artificial Sequence Synthetic peptide”
CSRWNAPSFYANDYW

SEQ ID NO: 114 - protein, 15 aa, Artificial Sequence
  source: /note=“Description of Artificial Sequence Synthetic peptide”
CSRWQLGGMYTHDYW

SEQ ID NO: 115 - protein, 15 aa, Artificial Sequence
  source: /note=“Description of Artificial Sequence Synthetic peptide”
CSRYGGNGMYANDYW

SEQ ID NO: 116 - protein, 15 aa, Artificial Sequence
  source: /note=“Description of Artificial Sequence Synthetic peptide”
CSRWERPGLYEHDYW

SEQ ID NO: 117 - protein, 15 aa, Artificial Sequence
  source: /note=“Description of Artificial Sequence Synthetic peptide”
CSRWRPDGFYEHDYW

SEQ ID NO: 118 - protein, 15 aa, Artificial Sequence
  source: /note=“Description of Artificial Sequence Synthetic peptide”
CSRWPSPSFYTNDYW

SEQ ID NO: 119 - protein, 15 aa, Artificial Sequence
  source: /note=“Description of Artificial Sequence Synthetic peptide”
CSRYQANGLYAYEYW

SEQ ID NO: 120 - protein, 15 aa, Artificial Sequence
  source: /note=“Description of Artificial Sequence Synthetic peptide”
CSRYGPNGMYQNDYW

SEQ ID NO: 121 - protein, 15 aa, Artificial Sequence
  source: /note=“Description of Artificial Sequence Synthetic peptide”
CSRWREPGLYEHDYW

SEQ ID NO: 122 - protein, 15 aa, Artificial Sequence
  source: /note=“Description of Artificial Sequence Synthetic peptide”
CSRYGNPGMYEHDYW

SEQ ID NO: 123 - protein, 15 aa, Artificial Sequence
  source: /note=“Description of Artificial Sequence Synthetic peptide”
CSRWDRPGLYEHDYW

SEQ ID NO: 124 - protein, 15 aa, Artificial Sequence
  source: /note=“Description of Artificial Sequence Synthetic peptide”
CSRWKEPGLYEHDYW

SEQ ID NO: 125 - protein, 15 aa, Artificial Sequence
  source: /note=“Description of Artificial Sequence Synthetic peptide”
CSRYGQPGMYEHDYW

SEQ ID NO: 126 - protein, 15 aa, Artificial Sequence
  source: /note=“Description of Artificial Sequence Synthetic peptide”
CSRYGQPGLYEHDYW

SEQ ID NO: 127 - protein, 15 aa, Artificial Sequence
  source: /note=“Description of Artificial Sequence Synthetic peptide”
CSRWDGPAFYELDYW

SEQ ID NO: 128 - protein, 15 aa, Artificial Sequence
  source: /note=“Description of Artificial Sequence Synthetic peptide”
CSRWRQDGLYEHDYW

SEQ ID NO: 129 - DNA, 33 bp, Artificial Sequence
  source: /note=“Description of Artificial Sequence Synthetic oligonucleotide”
tgtcaacagywytwctgywybccaccaacgttc

SEQ ID NO: 130 - protein, 11 aa, Artificial Sequence
  source: /note=“Description of Artificial Sequence Synthetic peptide”
  MOD_RES (4)..(4): Any amino acid
  VARIANT (5)..(5): /replace=“Phe”
  MOD_RES (7)..(7): Any amino acid
  SITE (1)..(11): /note=“Variant residues given in the sequence have no preference with respect to those in the annotations for variant positions”
CQQXYCXPPTF

SEQ ID NO: 131 - DNA, 33 bp, Artificial Sequence
  source: /note=“Description of Artificial Sequence Synthetic oligonucleotide”
gaacgttggtggvrwrcagwarwrctgttgaca

SEQ ID NO: 132 - DNA, 43 bp, Artificial Sequence
  source: /note=“Description of Artificial Sequence Synthetic oligonucleotide”
tgtagcaggtggggsggsgscggyttstacgcgttcgattatt

SEQ ID NO: 133 - protein, 14 aa, Artificial Sequence
  source: /note=“Description of Artificial Sequence Synthetic peptide”
  VARIANT (7)..(7): /replace=“Gly”
  VARIANT (9)..(9): /replace=“Leu”
  SITE (1)..(14): /note=“Variant residues given in the sequence have no preference with respect to those in the annotations for variant positions”
CSRWGGAGFYAFDY

SEQ ID NO: 134 - DNA, 43 bp, Artificial Sequence
  source: /note=“Description of Artificial Sequence Synthetic oligonucleotide”
aataatcgaacgcgtasaarccgscsccsccccacctgctaca